SEQUENCE LISTING (192 sequences)

SEQ ID NO: 1 - protein, 455 aa, Fragaria spp.
MEKIEVSIISKHTIKPSTSSSPLQPYKLTLLDQLTPPSYVPMVFFYPITGPAVFNLQTLADLRHALSETLTLYYPLSGRVKNNLYIDDFEEGVPYLEARVNCDMNDFLRLPKIECLNEFVPIKPFSMEAISDERYPLLGVQVNIFNSGIAIGVSVSHKLIDGRTSDCFLKSWCAVFRGSRDKIIHPNLSQAALLFPPRDDLPEKYARQMEGLWFVGKKVATRRFVFGAKAISVIQDEAKSESVPKPSRVQAVTSFLWKHLIATSRALTSGTTSTRLSIATQVVNIRSRRNMETVWDNAIGNLIWFAPAILELSHTTLEISDLKLCDLVNLLNGSVKQCNGDYFETFMGKEGYGSMCEYLDFQRTMSSMEPAPEIYLFTSWTNFFNQLDFGWGRTSWIGVAGKIESAFCNLTTLVPTPCDTGIEAWVNLEEEKMAMLEQDPQFLALASPKTLISRY

SEQ ID NO: 2 - protein, 452 aa, Fragaria sp.
MEKIEVSINSKHTIKPSTSSTPLQPYKLTLLDQLTPPAYVPIVFFYPITDHDFNLPQTLADLRQALSETLTLYYPLSGRVKNNLYIDDFEEGVPYLEARVNCDMTDFLRLRKIECLNEFVPIKPFSMEAISDERYPLLGVQVNVFDSGIAIGVSVSHKLIDGGTADCFLKSWGAVFRGCRENIIHPSLSEAALLFPPRDDLPEKYVDQMEALWFAGKKVATRRFVFGVKAISSIQDEAKSESVPKPSRVHAVTGFLWKHLIAASRALTSGTTSTRLSIAAQAVNLRTRMNMETVLDNATGNLFWWAQAILELSHTTPEISDLKLCDLVNLLNGSVKQCNGDYFETFKGKEGYGRMCEYLDFQRTMSSMEPAPDIYLFSSWTNFFNPLDFGWGRTSWIGVAGKIESASCKFIILVPTQCGSGIEAWVNLEEEKMAMLEQDPHFLALASPKTLI

SEQ ID NO: 3 - protein, 525 aa, Saccharomyces cerevisiae
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 4 - protein, 451 aa, Saccharomyces cerevisiae
MSEVSKWPAINPFHWGYNGTVSHIVGENGSIKLHLKDNKEQVDFDEFANKYVPTLKNGAQFKLSPYLFTGILQTLYLGAADFSKKFPVFYGREIVKFSDGGVCTADWLIDSWKKDYEFDQSTTSFDKKKFDKDEKATHPEGWPRLQPRTRYLKDNELEELREVDLPLVVILHGLAGGSHEPIIRSLAENLSRSGRFQVVVLNTRGCARSKITTRNLFTAYHTMDIREFLQREKQRHPDRKLYAVGCSFGATMLANYLGEEGDKSPLSAAATLCNPWDLLLSAIRMSQDWWSRTLFSKNIAQFLTRTVQVNMGELGVPNGSLPDHPPTVKNPSFYMFTPENLIKAKSFKSTREFDEVYTAPALGFPNAMEYYKAASSINRVDTIRVPTLVINSRDDPVVGPDQPYSIVEKNPRILYCRTDLGGHLAYLDKDNNSWATKAIAEFFTKFDELVV

SEQ ID NO: 5 - protein, 342 aa, Candida antarctica
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 6 - DNA, 92 bp, Bacillus subtilis
ctagtggttcgggatcgccgttgcaagtcttggaggaacaaaacaaagggatgatggctaaaatcaaatcatttttcggtgtacgttcctaa

SEQ ID NO: 7 - protein, 466 aa, Escherichia coli BL21
MPHSYDYDAIVIGSGPGGEGAAMGLVKQGARVAVIERYQNVGGGCTHWGTIPSKALRHAVSRIIEFNQNPLYSDHSRLLRSSFADILNHADNVINQQTRMRQGFYERNHCEILQGNARFVDEHTLALDCLDGSVETLTAEKFVIACGSRPYHPTDVDFTHPRIYDSDSILSMHHEPRHVLIYGAGVIGCEYASIFRGMDVKVDLINTRDRLLAFLDQEMSDSLSYHFWNSGVVIRHNEEYEKIEGCDDGVIMHLKSGKKLKADCLLYANGRTGNTDSLALQNIGLETDSRGQLKVNSMYQTAQPHVYAVGDVIGYPSLASAAYDQGRIAAQALVKGEATAHLIEDIPTGIYTIPEISSVGKTEQQLTAMKVPYEVGRAQFKHLARAQIVGMNVGTLKILFHRETKEILGIHCFGERAAEIIHIGQAIMEQKGGGNTIEYFVNTTFNYPTMAEAYRVAALNGLNRLF

SEQ ID NO: 8 - DNA, 51 bp, Artificial Sequence
  PCR primer
tgatatgactaataattagcggccgctagtattttcaggaatttctccaat

SEQ ID NO: 9 - DNA, 42 bp, Artificial Sequence
  PCR primer
ttgaaagacattaagaggggcaacgattaactatgataaatg

SEQ ID NO: 10 - DNA, 40 bp, Artificial Sequence
  PCR primer
ttaatcgttgcccctcttaatgtctttcaaactaaatacc

SEQ ID NO: 11 - DNA, 44 bp, Artificial Sequence
  PCR primer
actagtaaccatcacactgtaagataacctttgaattgttacgc

SEQ ID NO: 12 - DNA, 25 bp, Artificial Sequence
  PCR primer
attgcttgaatttcgtcttcaaaac

SEQ ID NO: 13 - DNA, 25 bp, Artificial Sequence
  PCR primer
tagataacatgatatccaaggtcgc

SEQ ID NO: 14 - DNA, 68 bp, Artificial Sequence
  PCR primer
gctcagtcctaggtataatgctagctttacttcatcttttgaagcgttttagagctagaaatagcaag

SEQ ID NO: 15 - DNA, 42 bp, Artificial Sequence
  PCR primer
gtcataaacttgctcaactgatatgatcttccataacttaac

SEQ ID NO: 16 - DNA, 51 bp, Artificial Sequence
  PCR primer
atataggtaatcgctttcataagggcccgatcggtcttgccttgctcgtcg

SEQ ID NO: 17 - DNA, 51 bp, Artificial Sequence
  PCR primer
tgatatgactaataattagcggccgcccccattagatactaaagaaactca

SEQ ID NO: 18 - DNA, 40 bp, Artificial Sequence
  PCR primer
ctaaactctttcaacaactacgtctctcatgtttgacctc

SEQ ID NO: 19 - DNA, 40 bp, Artificial Sequence
  PCR primer
atgagagacgtagttgttgaaagagtttagtatacaagtt

SEQ ID NO: 20 - DNA, 44 bp, Artificial Sequence
  PCR primer
actagtaaccatcacactgggtttgtaaaagcagtttattcatc

SEQ ID NO: 21 - DNA, 25 bp, Artificial Sequence
  PCR primer
gtttttgagagattaggtggaaagg

SEQ ID NO: 22 - DNA, 25 bp, Artificial Sequence
  PCR primer
tccctactgcatagattcctgcatt

SEQ ID NO: 23 - DNA, 68 bp, Artificial Sequence
  PCR primer
gctcagtcctaggtataatgctagctcaaaagtaaatgttaatgggttttagagctagaaatagcaag

SEQ ID NO: 24 - DNA, 51 bp, Artificial Sequence
  PCR primer
tgatatgactaataattagcggccgccagacagtggccttatcaataggag

SEQ ID NO: 25 - DNA, 40 bp, Artificial Sequence
  PCR primer
ttataatctttctacaactacttctttcatttatgctcct

SEQ ID NO: 26 - DNA, 40 bp, Artificial Sequence
  PCR primer
atgaaagaagtagttgtagaaagattataataatagataa

SEQ ID NO: 27 - DNA, 44 bp, Artificial Sequence
  PCR primer
actagtaaccatcacactgcctcttttttctttttcattgcttc

SEQ ID NO: 28 - DNA, 25 bp, Artificial Sequence
  PCR primer
tcagggacagcagttcatgtagtaa

SEQ ID NO: 29 - DNA, 25 bp, Artificial Sequence
  PCR primer
ttatggtaaagcctaatacaaaccc

SEQ ID NO: 30 - DNA, 68 bp, Artificial Sequence
  PCR primer
gctcagtcctaggtataatgctagcaagaaagttaatgtaagtgggttttagagctagaaatagcaag

SEQ ID NO: 31 - DNA, 51 bp, Artificial Sequence
  PCR primer
tgatatgactaataattagcggccgcaagacattaaagattctgcaacagc

SEQ ID NO: 32 - DNA, 42 bp, Artificial Sequence
  PCR primer
ttattctctttcaactactacatctttcattttttattcctc

SEQ ID NO: 33 - DNA, 40 bp, Artificial Sequence
  PCR primer
atgaaagatgtagtagttgaaagagaataattgaatttaa

SEQ ID NO: 34 - DNA, 44 bp, Artificial Sequence
  PCR primer
actagtaaccatcacactgatgcaagatacacaagctataggat

SEQ ID NO: 35 - DNA, 25 bp, Artificial Sequence
  PCR primer
ttttatgtcatagcaattgagggaa

SEQ ID NO: 36 - DNA, 25 bp, Artificial Sequence
  PCR primer
ttgttctttatatttttcattgggc

SEQ ID NO: 37 - DNA, 68 bp, Artificial Sequence
  PCR primer
gctcagtcctaggtataatgctagccttgcaaccttgtgtattgggttttagagctagaaatagcaag

SEQ ID NO: 38 - DNA, 51 bp, Artificial Sequence
  PCR primer
tgatatgactaataattagcggccgcaataactgatgtagccagtgcaact

SEQ ID NO: 39 - DNA, 40 bp, Artificial Sequence
  PCR primer
tcaattgcatctttctactacttctttcatttttaactcc

SEQ ID NO: 40 - DNA, 40 bp, Artificial Sequence
  PCR primer
atgaaagaagtagtagaaagatgcaattgatagaggaatg

SEQ ID NO: 41 - DNA, 44 bp, Artificial Sequence
  PCR primer
actagtaaccatcacactgaattttcttaccgctacaaagatca

SEQ ID NO: 42 - DNA, 25 bp, Artificial Sequence
  PCR primer
aatatttgctgaacttgatgacgta

SEQ ID NO: 43 - DNA, 25 bp, Artificial Sequence
  PCR primer
accatttaatacataatggtcacct

SEQ ID NO: 44 - DNA, 68 bp, Artificial Sequence
  PCR primer
gctcagtcctaggtataatgctagctgttaaatgatgcaagatgggttttagagctagaaatagcaag

SEQ ID NO: 45 - DNA, 51 bp, Artificial Sequence
  PCR primer
tgatatgactaataattagcggccgctgacaataatgtgtttaaatttcct

SEQ ID NO: 46 - DNA, 41 bp, Artificial Sequence
  PCR primer
gtggaaagtgtatatattaaaaggagctaaggatgaactat

SEQ ID NO: 47 - DNA, 40 bp, Artificial Sequence
  PCR primer
ttagctccttttaatatatacactttccacttataaaact

SEQ ID NO: 48 - DNA, 44 bp, Artificial Sequence
  PCR primer
actagtaaccatcacactgctattttaggatatgttgataatgc

SEQ ID NO: 49 - DNA, 25 bp, Artificial Sequence
  PCR primer
tttcattgattttatccatcttctt

SEQ ID NO: 50 - DNA, 25 bp, Artificial Sequence
  PCR primer
ttaatcctatttattcagatgaaga

SEQ ID NO: 51 - DNA, 68 bp, Artificial Sequence
  PCR primer
gctcagtcctaggtataatgctagcaactgtgctacttgaaaatagttttagagctagaaatagcaag

SEQ ID NO: 52 - DNA, 45 bp, Artificial Sequence
  PCR primer
tgatatgactaataattagcatcaaccagggaatttataagaagc

SEQ ID NO: 53 - DNA, 46 bp, Artificial Sequence
  PCR primer
agctgctaatttagtatcagtcttcatttaatggaatttacattaa

SEQ ID NO: 54 - DNA, 42 bp, Artificial Sequence
  PCR primer
taaatgaagactgatactaaattagcagctgaaaatgaaagt

SEQ ID NO: 55 - DNA, 46 bp, Artificial Sequence
  PCR primer
actagtaaccatcacactggcccaaaattcctttaacttgctcgta

SEQ ID NO: 56 - DNA, 25 bp, Artificial Sequence
  PCR primer
tattagtgcaaaatggctttatgac

SEQ ID NO: 57 - DNA, 25 bp, Artificial Sequence
  PCR primer
gcatctgaatccattattttagttt

SEQ ID NO: 58 - DNA, 68 bp, Artificial Sequence
  PCR primer
gctcagtcctaggtataatgctagcgatcatatattgctaaacatgttttagagctagaaatagcaag

SEQ ID NO: 59 - DNA, 45 bp, Artificial Sequence
  PCR primer
tgatatgactaataattagcgaagagaaagaaagcaaatattacc

SEQ ID NO: 60 - DNA, 40 bp, Artificial Sequence
  PCR primer
atagctcctatctttgtgttaaaaatgtgttaataataaa

SEQ ID NO: 61 - DNA, 41 bp, Artificial Sequence
  PCR primer
ttaacacatttttaacacaaagataggagctatatatgaaa

SEQ ID NO: 62 - DNA, 46 bp, Artificial Sequence
  PCR primer
actagtaaccatcacactggctttaacttcgatcctcttagcacct

SEQ ID NO: 63 - DNA, 25 bp, Artificial Sequence
  PCR primer
ccaagtgaaaaaattgaatttgata

SEQ ID NO: 64 - DNA, 25 bp, Artificial Sequence
  PCR primer
aatttttattaagaccctgccgcta

SEQ ID NO: 65 - DNA, 68 bp, Artificial Sequence
  PCR primer
gctcagtcctaggtataatgctagcggagctgcaacaactataaagttttagagctagaaatagcaag

SEQ ID NO: 66 - DNA, 45 bp, Artificial Sequence
  PCR primer
tgatatgactaataattagcgcttaatattgtctattgataccga

SEQ ID NO: 67 - DNA, 42 bp, Artificial Sequence
  PCR primer
gaataaaagatgattagatatgctcgaatttgattattataa

SEQ ID NO: 68 - DNA, 41 bp, Artificial Sequence
  PCR primer
aattcgagcatatctaatcatcttttattctatacacatta

SEQ ID NO: 69 - DNA, 46 bp, Artificial Sequence
  PCR primer
actagtaaccatcacactggcttataaatctttatggggcaagagg

SEQ ID NO: 70 - DNA, 25 bp, Artificial Sequence
  PCR primer
ttttccaaataatttgcttaatcct

SEQ ID NO: 71 - DNA, 25 bp, Artificial Sequence
  PCR primer
caagacaactaattatcgaaaaaca

SEQ ID NO: 72 - DNA, 68 bp, Artificial Sequence
  PCR primer
gctcagtcctaggtataatgctagctcacctccatattttcttctgttttagagctagaaatagcaag

SEQ ID NO: 73 - DNA, 45 bp, Artificial Sequence
  PCR primer
tgatatgactaataattagccagagctttgtgtaattgatgtaac

SEQ ID NO: 74 - DNA, 40 bp, Artificial Sequence
  PCR primer
tatattatcaagcaaataacccgtaagtaacaattaagct

SEQ ID NO: 75 - DNA, 40 bp, Artificial Sequence
  PCR primer
ttacttacgggttatttgcttgataatatatttaaatata

SEQ ID NO: 76 - DNA, 46 bp, Artificial Sequence
  PCR primer
actagtaaccatcacactggcctaatgaagccaatatggagaaact

SEQ ID NO: 77 - DNA, 25 bp, Artificial Sequence
  PCR primer
atgcaacataccggaaaaggtaaac

SEQ ID NO: 78 - DNA, 25 bp, Artificial Sequence
  PCR primer
aatagataggttgcaaaaggaaatg

SEQ ID NO: 79 - DNA, 68 bp, Artificial Sequence
  PCR primer
gctcagtcctaggtataatgctagctctcttaaatcactcattatgttttagagctagaaatagcaag

SEQ ID NO: 80 - DNA, 45 bp, Artificial Sequence
  PCR primer
tgatatgactaataattagcatttgtatgaaggtagcggttgagg

SEQ ID NO: 81 - DNA, 42 bp, Artificial Sequence
  PCR primer
taagatagtttagtttcctttacaaattaatgtattttttaa

SEQ ID NO: 82 - DNA, 40 bp, Artificial Sequence
  PCR primer
ttaatttgtaaaggaaactaaactatcttagaagacagac

SEQ ID NO: 83 - DNA, 46 bp, Artificial Sequence
  PCR primer
actagtaaccatcacactggccttaggaatgatattcgaaaaagca

SEQ ID NO: 84 - DNA, 29 bp, Artificial Sequence
  PCR primer
tctttccttataaagagtatcgtttctaa

SEQ ID NO: 85 - DNA, 26 bp, Artificial Sequence
  PCR primer
caattaaagtatgaacaaaagcatgg

SEQ ID NO: 86 - DNA, 68 bp, Artificial Sequence
  PCR primer
gctcagtcctaggtataatgctagcttcaaaattacaagcttcatgttttagagctagaaatagcaag

SEQ ID NO: 87 - DNA, 45 bp, Artificial Sequence
  PCR primer
tgatatgactaataattagcaatttcaagcataatggttatttca

SEQ ID NO: 88 - DNA, 40 bp, Artificial Sequence
  PCR primer
atgaaagcagctattaaaggcgctctttaatgtagcgccc

SEQ ID NO: 89 - DNA, 41 bp, Artificial Sequence
  PCR primer
ttaaagagcgcctttaatagctgctttcatagtatacctcc

SEQ ID NO: 90 - DNA, 46 bp, Artificial Sequence
  PCR primer
actagtaaccatcacactggctcttaatcagtcttcatttaatgga

SEQ ID NO: 91 - DNA, 25 bp, Artificial Sequence
  PCR primer
ggagatgctttttatgataaagaaa

SEQ ID NO: 92 - DNA, 25 bp, Artificial Sequence
  PCR primer
atatcattaatactgataatggcgg

SEQ ID NO: 93 - DNA, 68 bp, Artificial Sequence
  PCR primer
gctcagtcctaggtataatgctagccattaaccagtcttggtgcagttttagagctagaaatagcaag

SEQ ID NO: 94 - DNA, 45 bp, Artificial Sequence
  PCR primer
tgatatgactaataattagcgaaagcctcaggatggaaagatagt

SEQ ID NO: 95 - DNA, 43 bp, Artificial Sequence
  PCR primer
atggcaaataaaactttaaaaatgtgttaataataaaaatagc

SEQ ID NO: 96 - DNA, 40 bp, Artificial Sequence
  PCR primer
ttaacacatttttaaagttttatttgccataaaatcatcc

SEQ ID NO: 97 - DNA, 46 bp, Artificial Sequence
  PCR primer
actagtaaccatcacactggcagaaaacagaaaggattaactcaac

SEQ ID NO: 98 - DNA, 25 bp, Artificial Sequence
  PCR primer
gctgtgctggtatctaaaataaagg

SEQ ID NO: 99 - DNA, 24 bp, Artificial Sequence
  PCR primer
aggtgaaattattgtgttaggtga

SEQ ID NO: 100 - DNA, 68 bp, Artificial Sequence
  PCR primer
gctcagtcctaggtataatgctagcggaatagttacttttaagcagttttagagctagaaatagcaag

SEQ ID NO: 101 - DNA, 45 bp, Artificial Sequence
  PCR primer
tgatatgactaataattagcagctgacaaattaaaagttagtgaa

SEQ ID NO: 102 - DNA, 40 bp, Artificial Sequence
  PCR primer
ctaatttagcaacgcagtctttacagccataagtaaaacc

SEQ ID NO: 103 - DNA, 40 bp, Artificial Sequence
  PCR primer
atggctgtaaagactgcgttgctaaattagaaatataaac

SEQ ID NO: 104 - DNA, 46 bp, Artificial Sequence
  PCR primer
actagtaaccatcacactggcggaatgtttataccagtcctattca

SEQ ID NO: 105 - DNA, 25 bp, Artificial Sequence
  PCR primer
agaaaaatacagaaatgatcttcaa

SEQ ID NO: 106 - DNA, 24 bp, Artificial Sequence
  PCR primer
ggtacttaatttctctttggtttc

SEQ ID NO: 107 - DNA, 68 bp, Artificial Sequence
  PCR primer
gctcagtcctaggtataatgctagctgatatagattatgataatagttttagagctagaaatagcaag

SEQ ID NO: 108 - DNA, 45 bp, Artificial Sequence
  PCR primer
tgatatgactaataattagcaatttctgaacattttaaaggaaga

SEQ ID NO: 109 - DNA, 40 bp, Artificial Sequence
  PCR primer
tttaaatatattatccttataacttgccataaataccacc

SEQ ID NO: 110 - DNA, 40 bp, Artificial Sequence
  PCR primer
atggcaagttataaggataatatatttaaatataaacctc

SEQ ID NO: 111 - DNA, 46 bp, Artificial Sequence
  PCR primer
actagtaaccatcacactggcctaaggaagaaaacaaaatagccaa

SEQ ID NO: 112 - DNA, 25 bp, Artificial Sequence
  PCR primer
tacataaattctttccagaagggat

SEQ ID NO: 113 - DNA, 25 bp, Artificial Sequence
  PCR primer
gcccaattaaaaagtggagtagaag

SEQ ID NO: 114 - DNA, 68 bp, Artificial Sequence
  PCR primer
gctcagtcctaggtataatgctagcatatttaaatgttacccaccgttttagagctagaaatagcaag

SEQ ID NO: 115 - DNA, 25 bp, Artificial Sequence
  PCR primer
agatctccatggacgcgtgacgtcg

SEQ ID NO: 116 - DNA, 48 bp, Artificial Sequence
  PCR primer
tccatatgaccatgattacgttaaataaatttccttaaaaaacaatat

SEQ ID NO: 117 - DNA, 50 bp, Artificial Sequence
  PCR primer
atccccgggtaccgagctcgaattcgactaatcctccttatgatttaaaa

SEQ ID NO: 118 - DNA, 45 bp, Artificial Sequence
  PCR primer
tccatatgaccatgattacggttaagccactaccaggatcaatca

SEQ ID NO: 119 - DNA, 50 bp, Artificial Sequence
  PCR primer
atccccgggtaccgagctcgaattcttcttttcctcctcttacacacgca

SEQ ID NO: 120 - DNA, 45 bp, Artificial Sequence
  PCR primer
tccatatgaccatgattacgacaggagatagcaaatacagagcta

SEQ ID NO: 121 - DNA, 50 bp, Artificial Sequence
  PCR primer
atccccgggtaccgagctcgaattcaattttaataaccctcctcaaaata

SEQ ID NO: 122 - DNA, 45 bp, Artificial Sequence
  PCR primer
tccatatgaccatgattacgttagatcctaaaaaatatggcgtta

SEQ ID NO: 123 - DNA, 50 bp, Artificial Sequence
  PCR primer
atccccgggtaccgagctcgaattcaaattaaccacctcccaaattgaaa

SEQ ID NO: 124 - DNA, 47 bp, Artificial Sequence
  PCR primer
atataatttatgaaagggtggtttttatggagaaaattgaggtcagt

SEQ ID NO: 125 - DNA, 53 bp, Artificial Sequence
  PCR primer
cgactctagaggatccccgggtaccgaattctcaatatcttgaaattagcgtc

SEQ ID NO: 126 - DNA, 47 bp, Artificial Sequence; identical to SEQ ID NO: 124
  PCR primer

SEQ ID NO: 127 - DNA, 55 bp, Artificial Sequence
  PCR primer
cgactctagaggatccccgggtaccgaattcttaaattaaggtctttggagatgc

SEQ ID NO: 128 - DNA, 52 bp, Artificial Sequence
  PCR primer
atataatttatgaaagggtggtttttatgaatgaaatcgatgagaaaaatca

SEQ ID NO: 129 - DNA, 57 bp, Artificial Sequence
  PCR primer
cgactctagaggatccccgggtaccgaattcctaagggcctaaaaggagagctttgt

SEQ ID NO: 130 - DNA, 47 bp, Artificial Sequence
  PCR primer
atataatttatgaaagggtggtttttatgtcagaagtatctaagtgg

SEQ ID NO: 131 - DNA, 53 bp, Artificial Sequence
  PCR primer
cgactctagaggatccccgggtaccgaattcttatacaaccaactcgtcaaac

SEQ ID NO: 132 - DNA, 49 bp, Artificial Sequence
  PCR primer
atataatttatgaaagggtggtttttatgagaaaagtatttttaagatc

SEQ ID NO: 133 - DNA, 53 bp, Artificial Sequence
  PCR primer
cgactctagaggatccccgggtaccgaattcctaaggagtaacaattcctgag

SEQ ID NO: 134 - DNA, 51 bp, Artificial Sequence
  PCR primer
atataatttatgaaagggtggtttttatgaacgagatcgacgagaaaaacc

SEQ ID NO: 135 - DNA, 52 bp, Artificial Sequence
  PCR primer
cgactctagaggatccccgggtaccgaattcttacgggcctaacagcagcgc

SEQ ID NO: 136 - DNA, 52 bp, Artificial Sequence; identical to SEQ ID NO: 128
  PCR primer

SEQ ID NO: 137 - DNA, 57 bp, Artificial Sequence; identical to SEQ ID NO: 129
  PCR primer

SEQ ID NO: 138 - DNA, 46 bp, Artificial Sequence
  PCR primer
aatcataaggaggattagtcatgaatgaaatcgatgagaaaaatca

SEQ ID NO: 139 - DNA, 46 bp, Artificial Sequence
  PCR primer
gtgtaagaggaggaaaagaaatgaatgaaatcgatgagaaaaatca

SEQ ID NO: 140 - DNA, 46 bp, Artificial Sequence
  PCR primer
tgaggagggttattaaaattatgaatgaaatcgatgagaaaaatca

SEQ ID NO: 141 - DNA, 46 bp, Artificial Sequence
  PCR primer
atttgggaggtggttaatttatgaatgaaatcgatgagaaaaatca

SEQ ID NO: 142 - DNA, 51 bp, Artificial Sequence
  PCR primer
atccccgggtaccgagctcgaattcctaagggcctaaaaggagagctttgt

SEQ ID NO: 143 - DNA, 80 bp, Artificial Sequence
  PCR primer
agccatcatccctttgttttgttcctccaagacttgcaacggcgatcccgaaccactagtagggcctaaaaggagagctt

SEQ ID NO: 144 - DNA, 53 bp, Artificial Sequence
  PCR primer
gatccccgggtaccgagctcttaggaacgtacaccgaaaaatgatttgatttt

SEQ ID NO: 145 - DNA, 80 bp, Artificial Sequence
  PCR primer
gccatcatccctttgttttgttcctccaagacttgcaacggcgatcccgaaccactagtaattaaggtctttggagatgc

SEQ ID NO: 146 - DNA, 80 bp, Artificial Sequence
  PCR primer
ctagaggatccccgggtaccgagctcttaggaacgtacaccgaaaaatgatttgattttagccatcatccctttgttttg

SEQ ID NO: 147 - DNA, 45 bp, Artificial Sequence
  PCR primer
tgaggagggttattaaaattatgggtagcgaaattgccgcgctgg

SEQ ID NO: 148 - DNA, 44 bp, Artificial Sequence
  PCR primer
atttttctcatcgatttcattcatatggctgccgcgcggcacca

SEQ ID NO: 149 - DNA, 77 bp, Artificial Sequence
  PCR primer
actgaaaaagaaaaacgctgcgctgaaacagaaaattgccgcgctgaaacagatgagaaaaatgaaaactatggatg

SEQ ID NO: 150 - DNA, 79 bp, Artificial Sequence
  PCR primer
tttaggcccttaggagctcgaaagaggggtcaacgcgatgggtagcaaaatcgccgcactgaaaaagaaaaacgctgcg

SEQ ID NO: 151 - DNA, 51 bp, Artificial Sequence
  PCR primer
tcgactctagaggatccccgggtaccctattgttgattagctaacttcttg

SEQ ID NO: 152 - DNA, 77 bp, Artificial Sequence
  PCR primer
actgaaaaagaaaaacgctgcgctgaaacagaaaattgccgcgctgaaacagatgatgagatttacattaccaagag

SEQ ID NO: 153 - DNA, 80 bp, Artificial Sequence
  PCR primer
gttagctaatcaacaataggaggagggttattaaaattatgggtagcaaaatcgccgcactgaaaaagaaaaacgctgcg

SEQ ID NO: 154 - DNA, 51 bp, Artificial Sequence
  PCR primer
tcgactctagaggatccccgggtaccttaaaaatcaaccttatttccataa

SEQ ID NO: 155 - DNA, 45 bp, Artificial Sequence
  PCR primer
ttgtaaaacgacggccagtggtagactttaaggatggaacctttg

SEQ ID NO: 156 - DNA, 45 bp, Artificial Sequence
  PCR primer
ttctaacgcgtcacctaaagagatctccatggacgcgtgacgtcg

SEQ ID NO: 157 - DNA, 90 bp, Artificial Sequence
  PCR primer
aagaagaaaatagaaatttgacagctagctcagtcctaggtataatgctagcttggagatttcggacaacaagttttagagctagaaata

SEQ ID NO: 158 - DNA, 90 bp, Artificial Sequence
  PCR primer
tatttctagctctaaaacttgttgtccgaaatctccaagctagcattatacctaggactgagctagctgtcaaatttctattttcttctt

SEQ ID NO: 159 - DNA, 50 bp, Artificial Sequence
  PCR primer
tatgactaataattagcggccgcgatatgtagaggatgtgatcaagcttt

SEQ ID NO: 160 - DNA, 48 bp, Artificial Sequence
  PCR primer
ccaaatagctcttactccgccttcgctttaaatccctattgtccatct

SEQ ID NO: 161 - DNA, 45 bp, Artificial Sequence
  PCR primer
tggacaatagggatttaaagcgaaggcggagtaagagctatttgg

SEQ ID NO: 162 - DNA, 48 bp, Artificial Sequence
  PCR primer
gtaaccatcacactggcggccgctctgaaatatctgctccagcaacaa

SEQ ID NO: 163 - DNA, 90 bp, Artificial Sequence
  PCR primer
aagaagaaaatagaaatttgacagctagctcagtcctaggtataatgctagctcaagattaaaatatgtcgtgttttagagctagaaata

SEQ ID NO: 164 - DNA, 90 bp, Artificial Sequence
  PCR primer
tatttctagctctaaaacacgacatattttaatcttgagctagcattatacctaggactgagctagctgtcaaatttctattttcttctt

SEQ ID NO: 165 - DNA, 48 bp, Artificial Sequence
  PCR primer
tatgactaataattagcggccgctctgccagttgtcacttgaggtttt

SEQ ID NO: 166 - DNA, 50 bp, Artificial Sequence
  PCR primer
gctctgtatttgctatctcctgccattacttctcccatagctttatccat

SEQ ID NO: 167 - DNA, 49 bp, Artificial Sequence
  PCR primer
atttttcggtgtacgttcctaaggagttgtaggaattgtaaatccagtg

SEQ ID NO: 168 - DNA, 49 bp, Artificial Sequence
  PCR primer
gtaaccatcacactggcggccgccttgtgtttctctatctgatgctgga

SEQ ID NO: 169 - DNA, 46 bp, Artificial Sequence
  PCR primer
aaagctatgggagaagtaatggcaggagatagcaaatacagagcta

SEQ ID NO: 170 - DNA, 55 bp, Artificial Sequence
  PCR primer
gatttacaattcctacaactccttaggaacgtacaccgaaaaatgatttgatttt

SEQ ID NO: 171 - DNA, 90 bp, Artificial Sequence
  PCR primer
aagaagaaaatagaaatttgacagctagctcagtcctaggtataatgctagctatagcctttgtgaccgtgagttttagagctagaaata

SEQ ID NO: 172 - DNA, 90 bp, Artificial Sequence
  PCR primer
tatttctagctctaaaactcacggtcacaaaggctatagctagcattatacctaggactgagctagctgtcaaatttctattttcttctt

SEQ ID NO: 173 - DNA, 48 bp, Artificial Sequence
  PCR primer
tatgactaataattagcggccgccttgtagtcatttcagcgtgttccg

SEQ ID NO: 174 - DNA, 55 bp, Artificial Sequence
  PCR primer
tctttaattgccacttcttgaagcggccgctctcctttgaacataccctctccac

SEQ ID NO: 175 - DNA, 56 bp, Artificial Sequence
  PCR primer
ggagagggtatgttcaaaggagagcggccgcttcaagaagtggcaattaaagaggg

SEQ ID NO: 176 - DNA, 48 bp, Artificial Sequence
  PCR primer
gtaaccatcacactggcggccgcctcccatggtacctccttattgaaa

SEQ ID NO: 177 - DNA, 47 bp, Artificial Sequence
  PCR primer
tatgttcaaaggagagcggccgccaggagatagcaaatacagagcta

SEQ ID NO: 178 - DNA, 56 bp, Artificial Sequence
  PCR primer
ttgccacttcttgaagcggccgcttaggaacgtacaccgaaaaatgatttgatttt

SEQ ID NO: 179 - DNA, 90 bp, Artificial Sequence
  PCR primer
aagaagaaaatagaaatttgacagctagctcagtcctaggtataatgctagcaagtatcctaaattacaagggttttagagctagaaata

SEQ ID NO: 180 - DNA, 90 bp, Artificial Sequence
  PCR primer
tatttctagctctaaaacccttgtaatttaggatacttgctagcattatacctaggactgagctagctgtcaaatttctattttcttctt

SEQ ID NO: 181 - DNA, 47 bp, Artificial Sequence
  PCR primer
ctttgtgatatgactaataattaacacgagatatagacgaaaggagg

SEQ ID NO: 182 - DNA, 54 bp, Artificial Sequence
  PCR primer
tattccatgctgcttaaaatctgcggccgcattgctgccatcatttcctttagg

SEQ ID NO: 183 - DNA, 55 bp, Artificial Sequence
  PCR primer
taaaggaaatgatggcagcaatgcggccgcagattttaagcagcatggaataggc

SEQ ID NO: 184 - DNA, 48 bp, Artificial Sequence
  PCR primer
atccactagtaaccatcacactggcaaaacctggtgttaaacatcctt

SEQ ID NO: 185 - DNA, 47 bp, Artificial Sequence
  PCR primer
aatgatggcagcaatgcggccgccaggagatagcaaatacagagcta

SEQ ID NO: 186 - DNA, 56 bp, Artificial Sequence
  PCR primer
tgctgcttaaaatctgcggccgcttaggaacgtacaccgaaaaatgatttgatttt

SEQ ID NO: 187 - DNA, 90 bp, Artificial Sequence
  PCR primer
aagaagaaaatagaaatttgacagctagctcagtcctaggtataatgctagctcttggaactgaaattgaaagttttagagctagaaata

SEQ ID NO: 188 - DNA, 90 bp, Artificial Sequence
  PCR primer
tatttctagctctaaaactttcaatttcagttccaagagctagcattatacctaggactgagctagctgtcaaatttctattttcttctt

SEQ ID NO: 189 - DNA, 45 bp, Artificial Sequence
  PCR primer
ctttgtgatatgactaataattaaggggttagttacgcttcgcct

SEQ ID NO: 190 - DNA, 57 bp, Artificial Sequence
  PCR primer
tccaccacttactctatccttctgcggccgcataatggagcctcaatggaagcgata

SEQ ID NO: 191 - DNA, 57 bp, Artificial Sequence
  PCR primer
cgcttccattgaggctccattatgcggccgcagaaggatagagtaagtggtggattt

SEQ ID NO: 192 - DNA, 49 bp, Artificial Sequence
  PCR primer
atccactagtaaccatcacactgtatccaaagttgtaaacctcctaacg

(19) United States
(12) Patent Application Publication
(10) Pub. No.: US 2023/0340400 A1
(43) Pub. Date: Oct. 26, 2023

(54) MICROBIAL ESTER PRODUCTION

(71) Applicant: AUBURN UNIVERSITY, Aubum, AL (US)

(72) Inventors: Yi WANG, Aubum, AL (US); Jun FENG, Aubum, AL (US); Yuechao MA, Aubum, AL (US)

(73) Assignee: AUBURN UNIVERSITY, Aubum, AL (US)

(21) Appl. No.: 17/787,330

(22) PCT Filed: Jan. 21, 2021
(86) PCT No.: PCT/US2021/014301

§ 371 Date: Jun. 19, 2022

Related U.S. Application Data
Application No. PCT/US2021/014301 is a continuation-in-part of application No. PCT/US2020/066452, filed on Dec. 19, 2019.
(60) Provisional application No. 62/950,564, filed on Dec. 19, 2019.

(51) Int. Cl. C12N 9/10; C12N 1/20; C12N 15/52; C12P 7/62; C12N 15/74

(52) U.S. Cl. CPC C12N 1/20; C12N 9/1029; C12N 15/52; C12N 15/74; C12P 7/62; C12Y 203/01084; C12R 2001/145

(57) ABSTRACT

Microorganisms and microbial production methods for the biosynthesis of ester compounds are provided. Useful examples employ microorganisms that have been genetically modified to express alcohol acyltransferases, either from other species or that have been modified to increase their activity in catalyzing the esterification of alcohols. Additional useful examples employ microorganisms that have been genetically modified to express lipases, either from other species or that have been modified to increase their activity in catalyzing the esterification of organic acids. Additional modifications are presented that significantly increase ester production.

### CROSS REFERENCE TO RELATED APPLICATIONS

This application cites the priority of US62/950,564, filed on 19 Dec. 2019 (pending), which is incorporated herein by reference in its entirety.

### STATEMENT REGARDING FEDERALLY SPONSORED RESEARCH OR DEVELOPMENT

This invention was made with government support under: Grant No. 2016-31100-06001 awarded by the United States Department of Agriculture; Award DE-EE0008483 awarded by the United States Department of Energy; Competitive Grant no. 2018-67021-27715 by the United States Department of Agriculture; and Hatch project ALA014-1017025 by the United States Department of Agriculture. The government has certain rights in the invention.

In this context “government” refers to the government of the United States of America.

### BACKGROUND

### Field of the Disclosure

The present disclosure relates generally to industrial biotechnology.

### Background

Although tremendous efforts have been invested for biofuel and biochemical research, it is still challenging to generate robust microbial strains that can produce target products at desirable levels. Fatty acid esters, or mono-alkyl esters, can be used as valuable fuels such as diesel components or specialty chemicals for food flavoring, cosmetic and pharmaceutical industries. The US market demand for fatty acid esters could reach $4.99 billion by 2025.

Conventionally, esters are produced through Fischer esterification which involves high temperature and inorganic catalysts. The reaction consumes a large amount of energy and generates tremendous wastes, and thus is not environmentally friendly. On the other hand, ester production through biological routes is renewable and environmentally benign. Using current techniques, bioproduction of most of the esters is low, and is not economically competitive with environmentally damaging abiotic approaches.

There is therefore a need in the art for ester bioproduction approaches with higher production levels.

### SUMMARY

Strains of microorganism have been developed having vastly improved production of organic esters, by genetically modifying the microorganisms to introduce or enhance the activity of one or both of an alcohol acyltransferase and a lipase. This has resulted in some cases in 1-3 orders of magnitude increase in ester production, with specific examples being n-butyl acetate and n-butyl butyrate.

A first general embodiment is a modified microorganism capable of n-butyl acetate (BA) production, the microorganism capable of expressing an alcohol acyl transferase (AAT); and comprising an n-butanol synthesis pathway.

A second general embodiment is a modified microorganism capable of n-butyl butyrate (BB) production, the microorganism capable of expressing an AAT; and comprising a butyryl coenzyme A synthesis pathway and a butanol synthesis pathway.

A third general embodiment is a genetically modified Clostridium saccharoperbutylacetonicum having improved BA production and designated YM028P, having NCMA designation number 202012116, deposited on 16 Dec. 2020 at the National Center for Marine Algae and Microbiota at Bigelow Laboratory for Ocean Sciences, 60 Bigelow Drive, East Boothbay, ME 04544 USA.

A fourth general embodiment is a genetically modified Clostridium saccharoperbutylacetonicum having improved BB production and designated YM016PB, having NCMA designation number 202012115, deposited on 16 Dec. 2020 at the National Center for Marine Algae and Microbiota at Bigelow Laboratory for Ocean Sciences, 60 Bigelow Drive, East Boothbay, ME 04544 USA.

A fifth general embodiment is a genetically modified Clostridium saccharoperbutylacetonicum having improved BA production and designated FJ-1301, having NCMA designation number 202012114, deposited on 16 Dec. 2020 at the National Center for Marine Algae and Microbiota at Bigelow Laboratory for Ocean Sciences, 60 Bigelow Drive, East Boothbay, ME 04544 USA.

A sixth general embodiment is a method of ester production, comprising culturing a microorganism of the first through fifth general embodiments under conditions suitable to produce an ester.

A seventh general embodiment is an ester-containing composition comprising an ester compound that is the product of the method of the sixth embodiment.

The foregoing presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

### BRIEF DESCRIPTION OF THE DRAWINGS

FIG. 1 Engineering of solventogenic clostridia for fatty acid ester production. Top: Five strains out of four representative clostridial species were selected and evaluated as the host to be engineered for ester production in this study. We hypothesized that the different metabolic fluxes within different strains would make a big difference for the desirable ester production. The metabolic pathways of the four different species were represented in four different colors. Bottom: Fatty acid esters could be synthesized through two primary biological pathways: one is through the esterification of fatty acid and alcohol catalyzed by lipases, and the other is through the condensation of acyl-CoA and alcohol catalyzed by alcohol acyl transferases (AATs). Key genes in the pathway: pta, phosphotransacetylase; ack, acetate kinase; thl, thiolase; hbd, beta-hydroxybutyryl-CoA dehydrogenase; crt, crotonase; bcd, butyryl-CoA dehydrogenase; adh, alcohol dehydrogenase; adhE, Aldehyde-alcohol dehydrogenase; adc, acetoacetate decarboxylase; ctfAB, CoA transferase; ptb, phosphotransbutyrylase; buk, butyrate kinase; ald, aldehyde dehydrogenase.

FIG. 2 Screening of strains and enzymes for ester synthesis. (a) Schematic representation of devices and procedures for ester fermentation. (b) Heatmap results showed the ester production in various Clostridium strains with the overexpression of different enzymes. Empty plasmid: overexpression of pMTL82151 (for C. saccharoperbutylacetonicum N1-4-C, C. pasteurianum SD-1, C. tyrobutyricum cat1::adhE1 and cat1::adhE2) or pTJ1 (for C. beijerinckii 8052) as the control; EA: ethyl acetate; EB: ethyl butyrate; BA: butyl acetate; BB: butyl butyrate. Scale bar on the right is in g/L.

FIG. 3 Enhancing BA production through increasing the availability of acetyl-CoA (a & b) and dynamically expressing the atf1 gene (c & d). (a) Introduction of isopropanol synthesis pathway (shaded in purple) and deletion of thiolase genes. The pathways in purple arrows represent the ‘regeneration’ of actyl-CoA. There are five annotated genes encoding thiolase in C. saccharoperbutylacetonicum, only two (in red) of which could be deleted (see Supplementary materials). (b) The fermentation results for BA production with various mutant strains corresponding to the genetic manipulations in (a). The reported value is mean ± SD. (c) Four promoters associated with the biosynthesis of acetyl-CoA or alcohols in the pathway were selected to drive the expression of atf1. (d) The fermentation results for BA production with various mutant strains in which different promoters were used to drive the expression of atf1 as illustrated in (c). The reported value is mean ± SD. BA: butyl acetate. Key genes in the pathway: adh, alcohol dehydrogenase; bdh, butanol dehydrogenase; ctfAB, CoA transferase; sadh, secondary alcohol dehydrogenase; hydG, putative electron transfer protein; pfl, pyruvate formate lyase; ald, aldehyde dehydrogenase.

FIG. 4. Enhancing BA production through rational organization of the enzymes associated with BA synthesis. (a) Biological components evaluated in this study for rational organization of enzymes: (I) CC-Di-A and CC-Di-B tags, (II) PduA* scaffold, (III) MinD C-tag. (b) Schematic representation of assembling two of the three enzymes for BA synthesis with the CC-Di-A and CC-Di-B tags. (c) Schematic representation of organizing the three enzymes for BA synthesis onto the PduA* formed scaffold. (d) Schematic representation of using the MinD C-tag to draw ATF1 onto the cell membrane. (e) The fermentation results for BA production with various mutant strains corresponding to the genetic manipulations from (b)-(d). The reported value is mean ± SD. BA: butyl acetate.

FIG. 5 The deletion of the prophage genomes. (a) The gene cluster organization of the HM T prophage (P1) and another four putative prophages (P2-P5). (b) Comparison of the cell growth of prophage deleted mutants and the control N1-4-C strain. (c) Comparison of the butanol production in prophage deleted mutants and the control N1-4-C strain. (d) Comparison of the cell growth between ΔP1234 and ΔP12345. (e) Comparison of butanol production between ΔP1234 and ΔP12345; (f) Transmission election microscopy image of clostocin 0; (g) Transmission election microscopy image of the HM T prophage particles; (h, i) Cell growth profiles of ΔP1234 and ΔP12345 with the induction (at various OD₆₀₀) using mitomycin C at 2 or 4 µg/ml. “-” indicates that there was no cell lysis; “+” indicates that most of the cells were lysed. The value at the right side of the cell growth profile figure represents the actual OD₆₀₀ value at which mitomycin C (with the applied concentration included in the parentheses) was added for the induction.

FIG. 6 Fermentation results of the engineered strains for fatty acid ester production. (a) BA production in serum bottles using glucose (FJ-1201 and FJ-1301) or biomass hydrolysates (FJ-1201 (H)) as the substrate. (b) BA fermentation kinetics in FJ-1201 in 500-mL bioreactor using glucose as the substrate. (c) BA fermentation kinetics in FJ-1201 in 500-mL bioreactor using biomass hydrolysates as the substrate. (d) BB production in serum bottles using glucose (FJ-1202, FJ-1203, and FJ-1204) or biomass hydrolysates (FJ-1202 (H)) as the substrate. (e) BB fermentation kinetics in FJ-1202 in 500-mL bioreactor using glucose as the substrate. (f) BB fermentation kinetics in FJ-1202 in 500-mL bioreactor using biomass hydrolysates as the substrate. BA: butyl acetate; BB: butyl butyrate; Eth: ethanol; But: butanol.

FIG. 7. Techno-economic analysis of butyl acetate production from corn stover. a) process overview; b) total installed equipment cost; c) chemical production from each metric tonne (MT) of corn stover; d) butyl acetate production cost.

FIG. 8 Fermentation results in serum bottles with Clostridium saccharoperbutylacetonicum N1-4-C and the mutant strains. (a-h): ABE fermentation results for FJ-100 as compared to the control N1-4-C. The reported value is mean ± SD.

FIG. 9. Fermentation results in serum bottles with Clostridium saccharoperbutylacetonicum N1-4-C and the mutant strains with prophage deleted. (a-h): ABE fermentation results for ΔP1, ΔP2, ΔP3, ΔP4, ΔP1234 as compared to the control N1-4-C. (i-p): ABE fermentation results for ΔNP1, ΔNP2, ΔNP3, ΔNP4, ΔNP1234 as compared to the control N1-4-C. The reported value is mean ± SD.

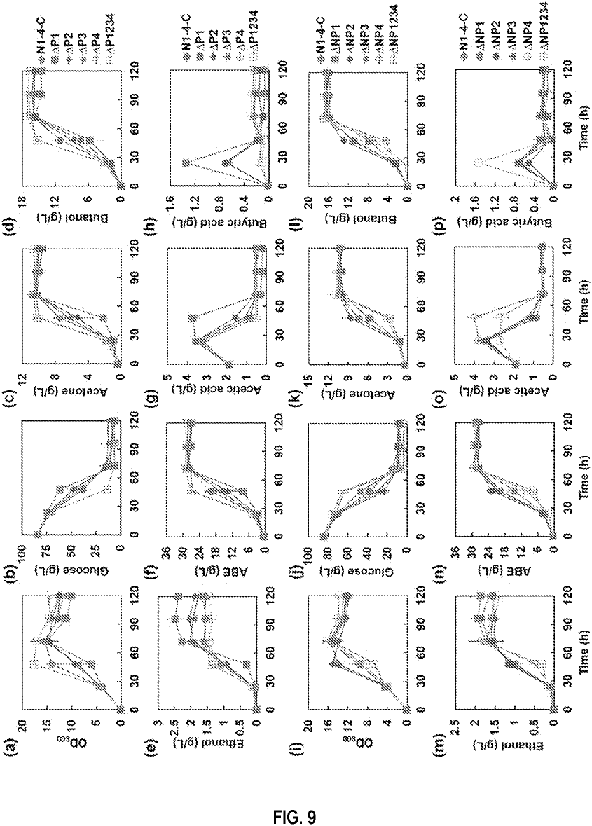

FIG. 10. Cell lysis in various prophage deletion mutants upon induction with different concentrations of mitomycin C. “I” indicates growth inhibition; “-” indicates that there was no cell lysis; “+” indicates that most of the cells were lysed; “++” indicates that all the cells were lysed.

FIG. 11. TEM picture of clostocin O. The magnification of (a) was 100x, while the magnification of (b) was 200x.

FIG. 12. Cell lysis in various prophage deletion mutants upon induction with different concentrations of mitomycin C. “-” indicates that there was no cell lysis; “+” indicates that most of the cells were lysed.

FIG. 13. HM T phage observed in the ΔP2345 strain upon induction. The magnification of (a, b) was 100x, while the magnification of (c, d) was 200x.

FIG. 14. ABE Fermentation results in serum bottles with Clostridium saccharoperbutylacetonicum ΔP1234 and ΔP12345. The reported value is mean ± SD.

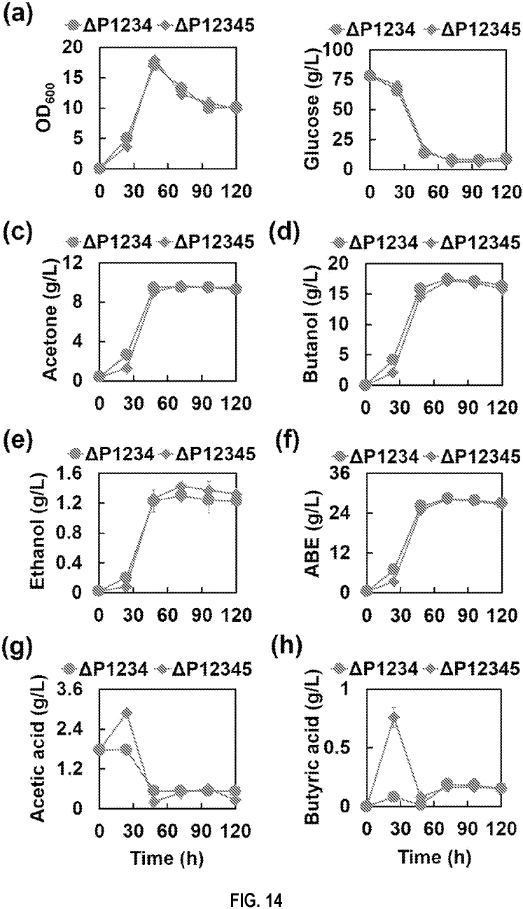

FIG. 15. ABE fermentation results in 500-mL bioreactors with Clostridium saccharoperbutylacetonicum ΔP1234 and ΔP12345. The reported value is mean ± SD.

FIG. 16. Sensitivity of butyl acetate production cost to different parameters. The numbers in brackets in Y-axis are the potential low, base and high values of each parameter.

### DETAILED DESCRIPTION

### Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

With reference to the use of the word(s) “comprise” or “comprises” or “comprising” in the foregoing description and/or in the following claims, unless the context requires otherwise, those words are used on the basis and clear understanding that they are to be interpreted inclusively, rather than exclusively, and that each of those words is to be so interpreted in construing the foregoing description and/or the following claims.

The term “consisting essentially of” means that, in addition to the recited elements, what is claimed may also contain other elements (steps, structures, ingredients, components, etc.) that do not adversely affect the operability of what is claimed for its intended purpose. Such addition of other elements that do not adversely affect the operability of what is claimed for its intended purpose would not constitute a material change in the basic and novel characteristics of what is claimed.

Terms such as “at least one of A and B” should be understood to mean “only A, only B, or both A and B.” The same construction should be applied to longer list (e.g., “at least one of A, B, and C”).

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. For biological systems, the term “about” refers to an acceptable standard deviation of error, preferably not more than 2-fold of a given value. Numerical quantities given herein are approximate unless stated otherwise, meaning that the term “about” or “approximately” can be inferred when not expressly stated.

The terminology used herein is for the purpose of describing particular embodiments only and is not intended to be limiting. As used herein, the singular forms “a”, “an” and “the” are intended to include the plural forms as well, unless the context clearly indicates otherwise.

The terms “first”, “second”, and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The term “nucleotide” as used herein refer to any such known groups, natural or synthetic. It includes conventional DNA or RNA bases (A, G, C, T, U), base analogs (e.g., inosine, 5-nitroindazole and others), imidazole-4-carboxamide, pyrimidine or purine derivatives (e.g., modified pyrimidine base 6H,8H-3,4-dihydropyrimido[4,5-c][1,2]oxazin-7-one (sometimes designated “P” base that binds A or G)) and modified purine base N6-methoxy-2,6-diaminopurine (sometimes designated “K” base that binds C or T), hypoxanthine, N-4-methyl deoxyguanosine, 4-ethyl-2′-deoxycytidine, 4,6-difluorobenzimidazole and 2,4-difluorobenzene nucleoside analogues, pyrene-functionalized LNA nucleoside analogues, deaza- or aza-modified purines and pyrimidines, pyrimidines with substituents at the 5 or 6 position and purines with substituents at the 2, 6 or 8 positions, 2-aminoadenine (nA), 2-thiouracil (sU), 2-amino-6-methylaminopurine, O-6-methylguanine, 4-thio-pyrimidines, 4-amino-pyrimidines, 4-dimethylhydrazine-pyrimidines, 0-4-alkyl-pyrimidines and hydrophobic nucleobases that form duplex DNA without hydrogen bonding. Nucleobases can be joined together by a variety of linkages or conformations, including phosphodiester, phosphorothioate or methylphosphonate linkages, peptide-nucleic acid linkages.

The term “polynucleotide” as used herein refers to a multimeric compound comprising nucleotides linked together to form a polymer, including conventional RNA, DNA, LNA, BNA, copolymers of any of the foregoing, and analogs thereof.

The term “nucleic acid” as used herein refers to a single stranded polynucleotide or a duplex of two polynucleotides. Such duplexes need not be annealed at all locations, and may contain gaps or overhangs.

The term “genetically modified” means that genetic material has been altered by human intervention. In the context of a self-replicating entity such as a cell or a virus, such alteration may have been performed on the self-replicating entity in question, or on an ancestor of the self-replicating entity from whom it acquired the alteration.

### Genetically Modified Microorganism

Strains of microorganism have been developed having vastly improved production of organic esters, by genetically modifying the microorganisms to introduce or enhance the activity of one or both of an alcohol acyl transferase and a lipase. This has resulted in some embodiments of the microorganism in 1-3 orders of magnitude increase in ester production, with specific examples being butyl acetate and butyl butyrate. AATs catalyze the addition of an acyl moiety from an acyl-coenzyme A (acyl-CoA) donor onto an alcohol acceptor. The efficiencies of various AATs vary widely depending on the alcohol and the acyl-CoA involved in the reaction. The work described herein discovered that some heterologous AATs in microorganisms catalyze the formation of butyl esters at extremely high efficiency, and that even higher efficiencies can be realized by further genetic modifications.

A first general embodiment of the microorganisms is capable of highly efficient BA production. This microorganism is capable of expressing the AAT, and has a functioning butanol synthesis pathway. Without wishing to be bound to a hypothetical model, it is believed that the AAT catalyzes transacetylation from acetyl-CoA to butanol to form the ester. The microorganism may also be characterized as having an acetyl-CoA synthesis pathway.

Although in theory any AAT could function to produce BA in this context, in preferred embodiments the AAT is one or more of: Vaat, Saat, Atf1, Eht1, and a functional homolog of any of the foregoing. The strawberry AAT Vaat (also found in banana) was elucidated by Beekwilder et al. (Plant Physiol. Vol. 135, 2004), having a reported amino acid sequence of SEQ ID NO: 1 (see also GenBank Accession No. AX025504.1). The strawberry AAT Saat was elucidated by Aharoni et al. (The Plant Cell, Vol. 12, 647-661, 2000 -incorporated herein by reference to teach the amino acid sequence), having a reported amino acid sequence of SEQ ID NO: 2 as reported in GenBank Accession No. AAG13130.1. The Saccharomyces cerevisiae AAT Atf1 was elucidated by Fuji et al. (Appl. Environ. Microbiol., Vol. 60, 2786-2792, 1994 - incorporated herein by reference to teach the amino acid sequence), having a reported amino acid sequence of SEQ ID NO: 3 as reported in NCBI Reference Sequence NP 015022.3. Yeast ethanol hexanoyl transferase (Eht1) was elucidated by Saerens et al., (J. Boil. Chem., Vol. 281, 2006- incorporated herein by reference to teach the amino acid sequence), having a reported amino acid sequence of SEQ ID NO: 4 as reported in UniProt Accession No. P40353.

Single or multiple copies of the AAT genes may be included. Various embodiments of the microorganism comprise a nucleic acid encoding one or more AATs, which may include any of the ATTs discussed above or their functional variants. A preferred embodiment of the microorganism comprises multiple nucleic acid sequences each encoding Atf1 or a functional homolog of Atf1. Another preferred embodiment of the microorganism comprises multiple genomic nucleic acid sequences each encoding Atf1 or a functional homolog of Atf1.

A second general embodiment is a microorganism capable of butyl butyrate (BB) production, the microorganism capable of expressing an AAT and comprising a butanol synthesis pathway and a butyryl coenzyme A synthesis pathway. It has been found that AATs can catalyze the condensation of butyryl coenzyme A and butanol to produce BB at high efficiency. Butyryl-coenzyme A is a four-carbon fatty acid that is the coenzyme A-activated form of butyric acid. The pathway may be endogenous or exogenous to the microorganism. One pathway was elucidated by Bennett and Rudolph (FEMS Microbiol. Rev. 17, 241-249, 1995) as including β-hydroxybutyryl-CoA dehydrogenase, crotonase, butyryl-CoA dehydrogenase and the α and β subunits of an electron transfer flavoprotein. In a preferred embodiment of the microorganism the AAT is Eht1 or a functional homolog of Eht1. In a further preferred embodiment of the microorganism the AAT is Saat or a functional homolog of Saat.

The microorganism may be constructed to contain a nucleic acid that encodes the AAT. Such nucleic acid may be genomic (part of the organism’s genome, such as a chromosome of a bacterium) or extra-genomic (such as part of an episome, for example a bacterial plasmid). The nucleic acid encoding the AAT may be a “canonical” sequence encoding the AAT, or it may be a degenerate variant of a canonical sequence. Degenerate variants can be constructed based on an understanding of the standard three nucleotide code and its corresponding amino acids. This correspondence is understood both for RNA and for DNA. Some organisms’ protein synthesis machinery employs atypical codons for certain amino acids, and this can be taken into account when designing nucleic acids that encode the AAT or its functional homologs.

Various embodiments of the microorganism may be eukaryotic or prokaryotic, such as bacteria, archaea, fungi, and protists. A preferred embodiment of the microorganism is a prokaryote. Prokaryotes have the advantage of rapid growth, easy cultivation, and simple genetics. The prokaryote may be a bacterium or an archaean. Bacteria have the advantage of being well understood model organisms with a wide diversity of metabolic niches. Archaea have the advantage of some specialized metabolic pathways and the ability in many cases to withstand extreme industrial conditions. An embodiment of the microorganism is a fermentative bacterium. A specific preferred embodiment of the microorganism is a bacterium of genus Clostridium, a well characterized group of fermentative anaerobic bacteria, capable of fermentation of a wide variety of substrates. In such embodiments the bacterium may be one of Clostridium saccharoperbutylacetonicum, Clostridium beijerinckii, Clostridium pasteurianum, and Clostridium tyrobutyricum. In a particularly preferred embodiment, the bacterium is Clostridium saccharoperbutylacetonicum.

Lipase B (CALB) from Candida antarctica (Pseudozyma Antarctica) catalyzes the formation of esters from fatty acids (including CoA fatty acids) and alcohols, and is commercially available for biochemical applications. Lipase B increases ester production in some embodiments of the microorganism. It has a reported amino acid sequence of SEQ ID NO: 5 as Uniprot Accession No. P41365. Some embodiments of the microorganism are capable of expressing Lipase B or a functional homolog of Lipase B. Such embodiments of the microorganism may be capable of such expression owing to the presence of a nucleic acid encoding Lipase B or a functional homolog of Lipase B. The nucleic acid may take various forms, including RNA (for example, as introduced mRNA) or DNA (for example, genomic DNA, episome DNA, or plasmid DNA). Some embodiments of the microorganism capable of expressing Lipase B have one or both of an acetic acid synthesis pathway and a butyric acid synthesis pathway. Butyric acid can form butyl esters with alcohols, and acetic acid can form acetyl esters with alcohols. Examples of suitable fermentatively produced alcohols include ethanol and butanol.

Enhancement of ester production can be achieved by reducing or eliminating the activity of the NADH-quinone oxidoreductase subunit G, which is a subunit of the electron transport chain complex I. The nuoG gene encodes the NADH-quinone oxidoreductase subunit G. Without wishing to be bound by any hypothetical model, it is believed that the availability of NADH is increased by reducing or eliminating the activity of the NADH-quinone oxidoreductase subunit G, leading to improved ester production. The nuoG in C. saccharoperbutylacetonicum is at locus tag Cspa_c47560 Some embodiments of the microorganism have partial or complete deletions of nuoG. The partial deletion may be sufficient to either completely inhibit expression, reduce the activity of the resulting polypeptide, or eliminate the activity of the resulting polypeptide. In a preferred embodiment the nuoG gene is deleted.

Ester production can also be increased by the expression of secondary alcohol dehydrogenase alone or in combination with the expression of the putative electron transfer protein hydG. One such suitable secondary alcohol dehydrogenase, that can convert acetone into isopropanol, is the one encoded by the sadh gene in C. beijerinckii B593 (Uniprot no. A0A1S8R6K8). The hydG gene in the same operon as sadh encodes a putative electron transfer protein. Some embodiments of the microorganism are capable of expressing one or both of hydG and sadh (or functional homologs thereof). Further embodiments of the microorganism comprise a sadh-hydG gene cluster. One or more of the foregoing may be heterologous genes. One example of a suitable sahH-hydG gene cluster is of clostridial origin, such as from C.beijerinckii B593.

The AAT may be expressed using various promoters and other regulatory elements. In this context the AAT is “operatively linked” to a promoter if the promoter controls the expression of the AAT. The promoter may be adjacent to the AAT gene, or it may be remote. Some embodiments of the microorganism comprise a constitutive promoter operatively linked to the AAT. P_{adh} is a promoter in Clostridium saccharoperbutylacetonicum, which controls expression of ahd, NADPH-dependent butanol dehydrogenase at locus tag Cspa_c04380. P_{ald} is a promoter in Clostridium saccharoperbutylacetonicum, which can sense the acidic state and switch cell metabolism from acidogenesis to solventogenesis. Operatively linking P_{adh} or P_{ald} to the AAT can increase ester production in some embodiments of the microorganism.. Some embodiments of the promoter are native to the microorganism

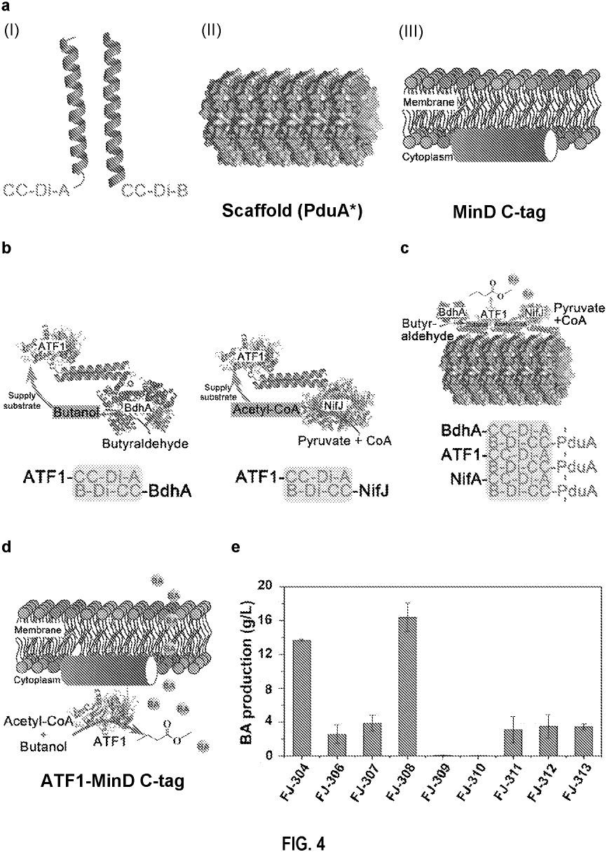

Additional advantages can be realized by localizing the AAT at the cell membrane. Without wishing to be bound by any hypothetical model, it is believed that the cell can be protected from toxic properties of the ester if the AAT is localized at the membrane. One such approach is to fuse the AAT with a membrane-associated molecule, such as MindD. MinD is a membrane-associated protein and the localization of MinD is mediated by an 8-12 residue C-terminal membrane-targeting sequence. It is an ubiquitous ATPase that plays a crucial role in selection of the division site in eubacteria and chloroplasts. The proteins with MinD C-terminal sequence are believed to be drawn to the cell membrane. Thus, the application of MinD C-tag can facilitate the secretion of target product and enhance its production by mitigating the intracellular toxicity as well as promoting the catalyzing process (FIG. 4a). In some embodiments of the microorganism the AAT is fused to a C-terminal membrane-targeting sequence of MinD. The AAT may be fused at any position on the AAT; in a preferred embodiment the AAT is fused at its C-terminal end to the C-terminal membrane-targeting sequence of MinD. The C-terminal membrane-targeting sequence of MinD may be part of a larger molecule, such as a whole MinD molecule. Some such embodiments of the microorganism may contain a nucleic acid encoding the AAT concatenated to a nucleic acid encoding MinD. An example of a suitable MinD tag is Bacillus subtilis MinD, having a canonical DNA sequence of SEQ ID NO: 6.

“Functional homologs” of a polypeptide will have some degree of identity with the wild type polypeptide. For example, it would be expected that most functional homologs having from 95-100% identity with the native polypeptide would retain at least some function. The likelihood that functionality would be retained by a homolog to the polypeptide with at least any of the following levels of sequence identity could be predicted: 70, 80, 90, 95, 99, and 99.5%. It is understood that the minimum desirable identity can be determined in some cases by identifying a known non-functional homolog to the polypeptide, and establishing that the minimum desirable identity must be above the identity between the polypeptide and the known non-functional identity. Persons having ordinary skill in the art will also understand that the minimum desirable identity can be determined in some cases by identifying a known functional homolog to the polypeptide, and establishing that the range of desirable identity must encompass the percent identity between the polypeptide and the known non-functional level of identity.

Deletions, additions and substitutions can be selected to generate a desired polypeptide functional homolog. For example, it is not expected that deletions, additions and substitutions in a non-functional region of a polypeptide would alter the polypeptide activity. Likewise conservative substitutions or substitutions of amino acids with similar properties is expected to be tolerated in a conserved region. Of course non-conservative substitutions in these regions would be expected to decrease or eliminate the polypeptide activity.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine. Conservative amino acid substitutions also encompass non-naturally occurring amino acid residues which are typically incorporated by chemical peptide synthesis rather than by synthesis in biological systems. These include peptidomimetics, and other reversed or inverted forms of amino acid moieties. It will be appreciated by those of skill in the art that nucleic acid and polypeptide molecules described herein may be chemically synthesized as well as produced by recombinant means.

Naturally occurring residues may be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe.

For example, conservative substitutions may involve the exchange of a member of one of these classes for a member of the same class.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (-0.4); threonine (-0.7); serine (-0.8); tryptophan (-0.9); tyrosine (-1.3); proline (-1.6); histidine (-3.2); glutamate (-3.5); glutamine (-3.5); aspartate (-3.5); asparagine (-3.5); lysine (-3.9); and arginine (-4.5). The importance of the hydropathic amino acid index in conferring interactive biological function on a protein is understood in the art (Kyte et al., J. Mol. Biol., 157:105-131, 1982). It is known that certain amino acids may be substituted for other amino acids having a similar hydropathic index or score and still retain a similar biological activity. In making conservative substitutions based upon the hydropathic index, the substitution of amino acids whose hydropathic indices are within +/- 2 may be used; in an alternate embodiment, the hydropathic indices are with +/- 1; in yet another alternate embodiment, the hydropathic indices are within +/- 0.5.

It is also understood in the art that the substitution of like amino acids can be made effectively on the basis of hydrophilicity. The greatest local average hydrophilicity of a polypeptide as governed by the hydrophilicity of its adjacent amino acids, correlates with a biological property of the protein. The following hydrophilicity values have been assigned to amino acid residues: arginine (+3.0); lysine (+3.0); aspartate (+3.0.+-.1); glutamate (+3.0.+-0.1); serine (+0.3); asparagine (+0.2); glutamine (+0.2); glycine (0); threonine (-0.4); proline (-0.5.+-0.1); alanine (-0.5); histidine (-0.5); cysteine (-1.0); methionine (-1.3); valine (-1.5); leucine (-1.8); isoleucine (-1.8); tyrosine (-2.3); phenylalanine (-2.5); tryptophan (-3.4). In making changes based upon similar hydrophilicity values, the substitution of amino acids whose hydrophilicity values are within +/- 2 may be used; in an alternate embodiment, the hydrophilicity values are with +/- 1; in yet another alternate embodiment, the hydrophilicity values are within +/- 0.5.

Desired amino acid substitutions (whether conservative or non-conservative) can be determined by those skilled in the art at the time such substitutions are desired. For example, amino acid substitutions can be used to identify important residues of the polypeptide, or to increase or decrease the affinity of the polypeptide with a particular binding target in order to increase or decrease the polypeptide activity.

Exemplary amino acid substitutions are set forth in Table 1.

TABLE 1

Conservative Amino Acid Substitutions

| | | |
|---|---|---|
| Original Amino Acid | Exemplary substitution | Preferred substitution |
| Ala | Val, Leu, Ile | Val |
| Arg | Lys, Gln, Asn | Lys |
| Asn | Glu | Glu |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Ile, Val, Met, Ala, Phe, Norleucine | Ile |
| Lys | Arg, 1,4-diaminobutyric acid, Gln, Asn | Arg |
| Met | Leu, Phe, Ile | Leu |
| Phe | Leu, Val, Ile, Ala, Tyr | Leu |
| Pro | Ala, Gly | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

For identifying suitable areas of the molecule that may be changed without destroying activity, one skilled in the art may target areas not believed to be important for activity. For example, when similar polypeptides with similar activities from the same species or from other species are known, one skilled in the art may compare the amino acid sequence of a given polypeptide to such similar polypeptides. With such a comparison, one can identify residues and portions of the molecules that are conserved among similar polypeptides. It will be appreciated that changes in areas of the polypeptide that are not conserved relative to such similar polypeptides would be less likely to adversely affect the biological activity and/or structure of the polypeptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Further advantages can be realized by curing the microorganism of viruses in its genome. In this context “cure” means to introduce a mutation that prevents the virus from replicating or otherwise expressing viral genes. An example of curing is entirely deleting the viral genome from the microorganism. Another example is partially deleting the viral genome form the microorganism, to such an extent that viral replication is not possible. Another example is mutating a bacteriophage integrase gene, preventing functioning phage integrase from being expressed. The mutation may be an entire deletion, a partial deletion, a substitution, or an insertion so long as functioning bacteriophage integrase in not expressed. Some embodiments of the microorganism are a bacterium that has been cured of one, some, or all prophages. A preferred embodiment of the bacterium is a Clostridium spp. that has been cured of all native prophages. In a further preferred embodiment, the microorganism is a bacterium of genus Clostridium which has been cured of one or more of prophages P1, P2, P3, P4, and P5 (described further below in the examples).

The rex gene was identified as a redox-sensing transcriptional repressor, which modulates transcription in response to changes in cellular NADH/NAD+ redox state, and represses the transcription of genes related to butanol synthesis. It was originally identified by Wietzke and Bahl (Appl. Microbiol. Biotechnol. 96(3): 749-61, 2012) (C. saccharoperbutylacetonicum: Cspa_c04320). Some embodiments of the microorganism do not express a functional redox-sensing transcriptional repressor. This lack of expression may be native to the organism, or induced through genetic modification. Such genetic modification may take any suitable form, such as a complete deletion, a partial deletion, an insertion, or a substitution.

Further advantages can be realized in embodiments of the microorganism capable of expressing soluble pyridine nucleotide transhydrogenase (SthA), or a functional homolog thereof, for converting NADPH to NADH. One example of a suitable SthA is from E. coli BL21 (SEQ ID NO: 7).

Further advantages can be realized in embodiments of the microorganism that do not express a functional cftA1-ctfB1 gene cluster (encoding acetoacetyl-CoA:acetate/butyrate:CoA transferase). Acetoacetyl-CoA:acetate/butyrate:CoA transferase transfers CoA group from acetoacetyl-CoA to either acetate or butyrate, generating acetyl-CoA or butyryl-CoA respectively. The ctfA1-ctfB1 gene cluster catalyzes the following two reactions: acetoacetyl-CoA + acetate ➔ acetoacetate + acetyl-CoA, acetoacetyl-CoA + butyrate ➔ acetoacetate + butyryl-CoA. The ctfA1 (atoD1): Gene ID in C. saccharoperbutylacetonicum is Cspa_c21000 csr:Cspa_c21000 K01034 acetate CoA/acetoacetate CoA-transferase alpha subunit [EC:2.8.3.8 2.8.3.9] | (GenBank) atoD1; acetate CoA-transferase subunit alpha (N); and ctfB1: Gene ID in C. saccharoperbutylacetonicum is Cspa_c21010 csr:Cspa_c21010 K01035 acetate CoA/acetoacetate CoA-transferase beta subunit [EC:2.8.3.8 2.8.3.9] | (GenBank) ctfB1; butyrate--acetoacetate CoA-transferase subunit B (N). This lack of expression may be native to the organism, or induced through genetic modification. Such genetic modification may take any suitable form, such as a complete deletion, a partial deletion, an insertion, or a substitution.

A specific embodiment of the microorganism is a genetically modified Clostridium saccharoperbutylacetonicum having improved BA production and designated YM028P, having NCMA designation number 202012116, deposited on 16 Dec. 2020 at the National Center for Marine Algae and Microbiota at Bigelow Laboratory for Ocean Sciences, 60 Bigelow Drive, East Boothbay, ME 04544 USA.

A specific embodiment of the microorganism is a genetically modified Clostridium saccharoperbutylacetonicum having improved BB production and designated YM016PB, having NCMA designation number 202012115, deposited on 16 Dec. 2020 at the National Center for Marine Algae and Microbiota at Bigelow Laboratory for Ocean Sciences, 60 Bigelow Drive, East Boothbay, ME 04544 USA.

A further specific embodiment of the microorganism is a genetically modified Clostridium saccharoperbutylacetonicum having improved BA production and designated FJ-1301, having NCMA designation number 202012114, deposited on 16 Dec. 2020 at the National Center for Marine Algae and Microbiota at Bigelow Laboratory for Ocean Sciences, 60 Bigelow Drive, East Boothbay, ME 04544 USA.

### Method of Ester Production

A method of ester production is disclosed, comprising culturing any one of the microorganisms above under conditions suitable to produce an ester. In a preferred embodiment of the method the ester produced is one or both of BA and BB. It has been observed that organisms that have been modified as described above can generate extremely large amount of BA and BB at unprecedented levels of efficiency.

Culturing may occur in the presence of a suitable carbon source and a suitable energy source, depending on the organism. Some embodiments of the culture medium contain glucose, which is widely used by heterotrophic organisms as both a source of carbon and energy. The glucose can be part of a defined medium or part of a complex medium. Numerous such glucose media are known in the art, some of which are cataloged in R. Atlas, Handbook of Microbiological Media, Fourth Edition, CRC Press (2010). In a preferred embodiment the medium comprises a biomass hydrolysate. Defined media have the advantage of being of controlled composition, whereas a complex medium such as biomass hydrolysate are less expensive to produce and require no supplementation. One suitable example of hydrolysate is a corn stover hydrolysate, such as one prepared according to the method of D. Humbird et al., “Process design and economics for biochemical conversion of lignocellulosic biomass to ethanol:dilute-acid pretreatment and enzymatic hydrolysis of corn stover” (No. NREL/TP-5100-47764) National Renewable Energy Lab.(NREL), Golden, CO (United States) (2011) - which is incorporated by reference to the extent necessary to describe and enable such hydrolysates.

Culturing should be conducted at a temperature conducive to microbial metabolism, which will vary depending on the microorganism involved. Often mesophilic organisms are used in ester fermentations, and in such cases culturing occurs at mesophilic temperatures. Higher temperatures can accelerate metabolic processes, and it is contemplated that thermophilic organisms could be used as well, in which case culturing would preferably be at thermophilic temperatures.

Ester production is generally a fermentative process. To encourage fermentation the culturing may be conducted under low oxygen conditions. Conditions may be hypoxic or anoxic, as necessary to create anaerobic conditions conducive to fermentation. It is possible that the microorganism could be engineered to ferment under aerobic conditions as well.

Such fermentation processes have been observed to generate extremely high concentrations of BA in culture, in excess of 25 g/L. Some embodiments of the culture method produce at least 1.5 g BA/L of culture. Further embodiments have been observed to produce at least 5, 7.5, 10, 12.5, 13, 14, 15, 16, 17, 18, 19, 20, or 25 g BA/L of culture. Higher concentrations of BB production have been observed than previous known methods. Some embodiments of the culture method produce at least 0.1 g BB/L of culture. Further embodiments of the culture method produce at least 0.2, 0.3, 0.4, 0.5, 0.6, 0.7, 0.8, 0.9, 1.0, 1.1, 1.2, 1.3, 1.4, 1.5. or 1.6 g BB/L of culture.

### Working Example: Renewable Fatty Acid Ester Production in Clostridium

An earlier version of this work was published on bioRxiv on 20 Aug. 2020 (available at https://www.biorxiv.org/content/10.1101/2020.03.29.014746v1.full) and is incorporated herein by reference in its entirety. The terms “we” and “our” used below refer to authors of the manuscript and persons who contributed to the work, some of whom might not be considered “inventors” of what is claimed under the laws of certain countries; the use of such terms is neither a representation nor an admission that any person is legally an inventor of what is claimed.

### Abstract

Production of renewable chemicals through biological routes is considered as an urgent solution for fossil energy crisis. However, end product toxicity inhibits microbial performance and is a key bottleneck for biochemical production. To address this challenge, here we report an example of biosynthesis of high-value and easy-recoverable derivatives to alleviate endproduct toxicity and enhance bioproduction efficiency. By leveraging the natural pathways in solventogenic clostridia for co-producing acyl-CoAs, acids and alcohols as precursors, through rational screening for host strains and enzymes, systematic metabolic engineering—including rational organization of ester-synthesizing enzymes inside of the cell, and elimination of putative prophages, we developed strains that can produce 25.3 g/L butyl acetate and 1.6 g/L butyl butyrate respectively, which were both the unprecedented levels in microbial hosts. Techno-economic analysis indicated a production cost of $986 per metric tonne for butyl acetate production from corn stover compared to the market price of $1,200-1,400 per metric tonne of butyl acetate, suggesting the economic competitiveness of the bioprocess.

### Main

Although tremendous efforts have been invested for biofuel and biochemical research, it is still challenging to generate robust microbial strains that can produce target products at desirable levels¹. One key bottleneck is the intrinsic toxicity of end products to host cells². Therefore, the production of high-value bioproducts which can be easily recovered from fermentation might be a solution to tackle the bottleneck in bioproduction. Fatty acid esters, or mono-alkyl esters, can be used as valuable fuels such as diesel components or specialty chemicals for food flavoring, cosmetic and pharmaceutical industries³. It is projected that the US market demand for fatty acid esters could reach $4.99 billion by 2025⁴. In addition, esters, with fatty acid and alcohol moieties, are generally hydrophobic and can easily separate from fermentation; thus the production of ester can help mitigate end product toxicity to host cells and efficient bioproduction can be achieved.

Esters are produced through Fischer esterification which involves high temperature and inorganic catalysts^{5,6}. The reaction consumes a large amount of energy and generates tremendous wastes, and thus is not environmentally friendly⁵. On the other hand, ester production through biological routes is becoming more and more attractive because it is renewable and environmentally benign. There are at least two known biological pathways for ester production: one is through esterification of fatty acid and alcohol catalyzed by lipases⁷, and the other is based on condensation of acyl-CoA and alcohol catalyzed by alcohol acyl transferases (AATs)⁵. Previously, lipases from bacteria or fungi have been employed for catalyzing esterification⁸.

In this study, we report highly efficient fatty acid ester production to unprecedented levels using engineered clostridia. We selected solventogenic clostridia to take advantage of their natural pathways for co-producing acyl-CoAs (acetyl-CoA and butyryl-CoA), fatty acids (acetate and butyrate), and alcohols (ethanol and butanol), either as intermediates or end products; we hypothesized that clostridia can be excellent microbial platforms to be engineered for efficient ester production by introducing heterologous AATs and/or lipase genes. Indeed, through screening for host strains (from four well-known clostridial species) and enzymes (alcohol acyl transferases and lipase), systematic metabolic engineering—including organization of ester-synthesizing enzymes inside of the cell, and elimination of putative prophages, we ultimately obtained two strains which can produce 25.3 g/L butyl acetate (BA) and 1.6 g/L BB respectively in extractive batch fermentations. These production levels were both highest in record to the best of our knowledge.

### Results

### Screening of Host Strains and Genes for Ester Production

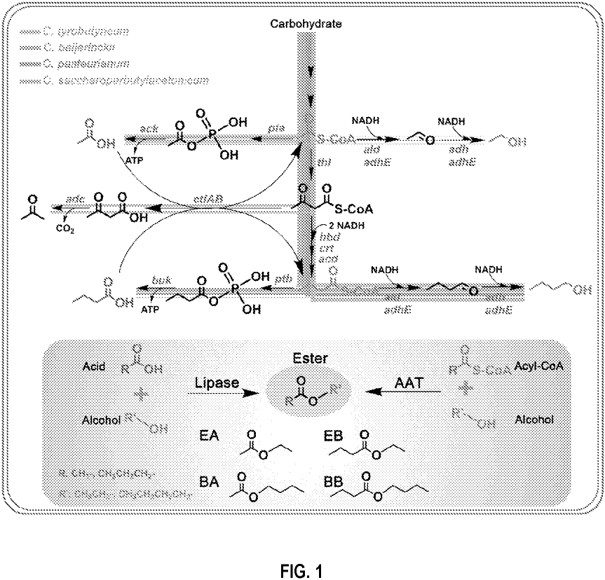

We considered clostridia as model platforms for ester production thanks to their intrinsic intermediates (fatty acids, acyl-CoAs, and alcohols) serving as precursors for ester biosynthesis (FIG. 1). We hypothesized that different flux levels of these precursors within various clostridial strains would make a big difference for the specific type(s) of ester production. Therefore, we selected five strains (from four representative species) including C. tyrobutyricum Δcat1::adhE1¹¹, C. tyrobutyricum Δcat1::adhE2¹¹, C. pasteurianum SD-1¹², C. saccharoperbutylacetonicum N1-4-C¹³ and C. beijerinckii NCIMB 8052¹⁴ to evaluate their capabilities for ester production through metabolic engineering (Table S1). We included both C. tyrobutyricum Δcat1::adhE1 and Δcat1::adhE2 here because they produce different levels (and thus ratios) of butanol and ethanol^{11,15}. Esters can be synthesized either through esterification of acid and alcohol catalyzed by lipase, or through condensation of acyl-CoA and alcohol catalyzed by AATs (FIG. 1). Previously, lipase B (CALB) from Candida antarctica has been employed for efficient ester production through esterification^{9,16}. In addition, four AATs including VAAT^{17,18}, SAAT¹⁷, ATF1^{3,10},_ ENREF_29 EHT1¹⁹ have been recruited for ester production in various hosts^{5,20-22}. Therefore, here, we evaluated all these genes in our clostridial hosts for ester production.

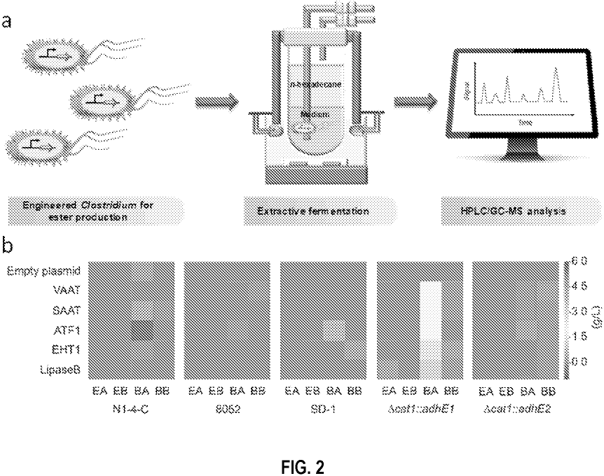

Six plasmids (pMTL-Pcat-vaat, pMTL-P_{cat}-saat, pMTL-P_{cat}-atf1, pMTL-P_{cat}-eht1, pMTL-P_{cat}-lipaseB as well as pMTL82151 as the control) were individually transformed into C. saccharoperbutylacetonicum N1-4-C, C. pasteurianum SD-1, C. tyrobutyricum cat1::adhE1 and cat1::adhE2 respectively. While pTJ1-P_{ca}t-vaat, pTJ1-P_{ca}t-saat, pTJ1-P_{cat}-atf1, pTJ1-P_{cat}-ehtl and pTJ1-P_{cat}-lipaseB as well as pTJ1 were transformed into C. beijerinckii 8052. Fermentations were performed (FIG. 2a), and results were shown in FIG. 2b. Four types of esters were detected: EA, BA, ethyl butyrate (EB) and BB. Interestingly, control strains with the empty plasmid (pMTL82151 or pTJ1) also produced noticeable EA, BA and BB. This could be because: 1) the endogenous lipase in clostridia can catalyze ester production¹³; 2) catP on pMTL82151 encoding a chloramphenicol acetyltransferase (belonging to the same class of enzymes as AATs) has AAT activities^{5,23}.

Based on the results, it could be concluded that ATF1 is more favorable for BA production. All strains with atf1 produced higher levels of BA compared to the same strain but with the overexpression of other genes (FIG. 2b). While VAAT, SAAT and EHT1 seemed to have better activities for BB production. Among all the strains, C. saccharoperbutylacetonicum FJ-004 produced the highest titer of 5.5 g/L BA. This is the highest BA production that has been reported through microbial fermentation to the best of our knowledge. C. acetobutylicum CaSAAT (with the overexpression of saat from Fragaria xananassa) was reported to produce 8.37 mg/L BA²². The highest BB production of 0.3 g/L was observed in C. pasteurianum J-5 with the overexpression of eht1. This is also the highest BB production reported so far directly from glucose with engineered microorganisms to the best of our knowledge, which is significantly higher than the recently reported 50.07 mg/L in an engineered C. acetobutylicum²². Some of our engineered strains could also produce small amount of EB with the highest of 0.02 g/L been observed in C. pasteurianum J-5. With the lipaseB overexpression, C. tyrobutyricum JZ-6 could generate 0.3 g/L EA. This was significantly higher than other strains tested in this work (mostly < 0.01 g/L). The mother strain C. tyrobutyricum Δcat1::adhE1 could produce 20.8 g/L acetate and 5.3 g/L ethanol (precursors for EA synthesis) during a batch fermentation, which might have enabled high-level EA production in C. tyrobutyricum JZ-6¹¹.

The production levels of BA and BB achieved above are both significantly higher than the previously reported in microbial hosts to the best of our knowledge. In comparison, the BA level is much higher than BB level, and thus has greater potentials towards economic viability. Therefore, in the following steps, we primarily focused on systematic metabolic engineering of the strain for further enhanced BA production.

### Deletion of nuoG Increased BA Production

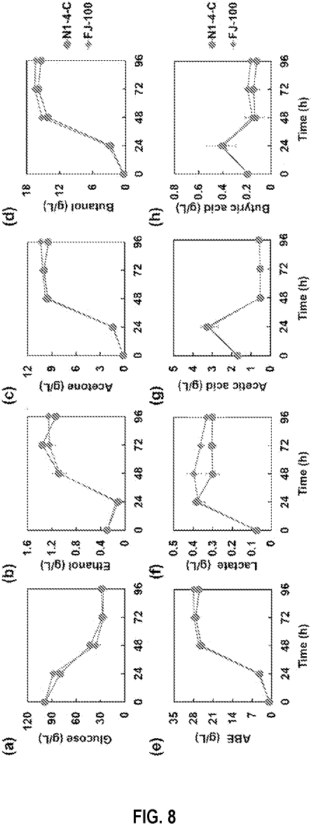

Enhancement of the pool of precursors is one common strategy to improve the production of targeted bioproduct. Butanol and acetyl-CoA are the two precursors for BA production. Therefore, we set out to increase butanol synthesis to improve BA production. The nuoG gene encodes the NADH-quinone oxidoreductase subunit G, which is a subunit of the electron transport chain complex I²⁵. NADH-quinone oxidoreductase can oxidize NADH to NAD⁺ and transfer protons from cytoplasm to periplasm to form a proton gradient between periplasm and cytoplasm, which can then contribute to the energy conversion²⁵. In this study, we hypothesized that by deleting nuoG, BA production would be boosted because of the potentially increased butanol production. Thus, we deleted nuoG (Cspa_c47560) in N1-4-C and generated FJ-100. Further, FJ-101 was constructed based on FJ-100 for BA production. Results demonstrated that, although butanol production in FJ-100 was only slightly improved (16.5 g/L vs. 15.8 g/L in N1-4-C; FIG. 8), BA production in FJ-101 was remarkably enhanced compared to FJ-004 (7.8 g/L vs. 5.5 g/L). Based on our experiences, because C. saccharoperbutylacetonicum N1-4 (or N1-4-C) mother strain can naturally produce very high level butanol, it was generally very difficult to further improve butanol production in C. saccharoperbutylacetonicum through simple metabolic engineering strategies²⁷. This is likely the case in FJ-100 (comparing to N1-4-C). However, the increased NADH availability with nuoG deletion in FJ-101 would enable an enhanced ‘instant’ flux/availability of butanol which would serve as a precursor for BA production and thus enhance BA production in FJ-101. In this sense, the total butanol generated during the process (including the fraction serving as the precursor for BA production and the other fraction as the end product) in FJ-101 would be actually much higher than in FJ-004.

### Enhancement of Acetyl-CoA Availability to Improve BA Production

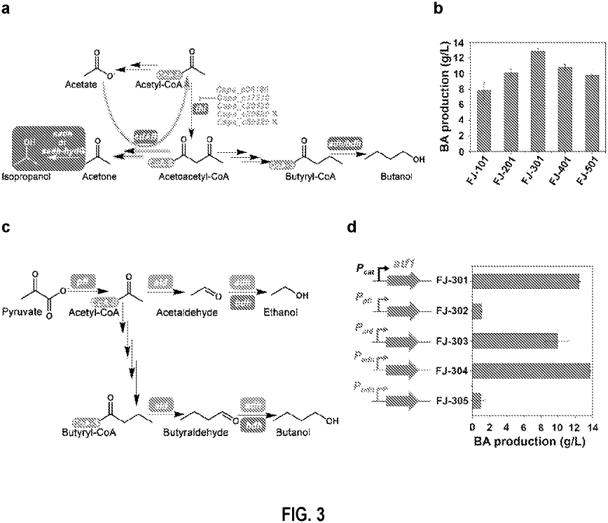

At the end of fermentation with FJ-101, there was still 7.6 g/L of butanol remaining. This suggested that limited availability of intracellular acetyl-CoA was likely the bottleneck for the further improvement of BA production. To enhance the availability of acetyl-CoA, we firstly introduced isopropanol synthesis (from acetone) pathway (FIG. 3A). We hypothesized that this could pull flux from acetone to isopropanol, and thus boost the transferring of CoA from acetoacetyl-CoA to acetate driven by the CoA transferase, resulting in increased instant availability of acetyl-CoA. The sadh gene in C. beijerinckii B593 encoding a secondary alcohol dehydrogenase can convert acetone into isopropanol²⁸. The hydG gene in the same operon as sadh encodes a putative electron transfer protein. In this study, either sadh alone or the sadh-hydG gene cluster was integrated into the chromosome of FJ-100, generating FJ-200 and FJ-300, respectively. Further, by introducing pMTL-cat-atf1, FJ-201 and FJ-301 were obtained. Compared to FJ-101, about 50% of the acetone could be converted into isopropanol in FJ-201, while ~95% of the acetone in FJ-301 could be converted into isopropanol. The total titers of acetone plus isopropanol in FJ-301 and FJ-201 were 6.6 and 5.1 g/L respectively, both of which were higher than 4.8 g/L acetone in FJ-101. More significantly, BA production in FJ-201 and FJ-301 has been remarkably increased compared to FJ-101, and reached 10.1 and 12.9 g/L, respectively, likely due to enhanced ‘regeneration’ of acetyl-CoA as described above (FIG. 3b).

### Dynamic Expression of Atf1 Enhanced BA Production

In our engineered strain, BA is synthesized through condensation of butanol and acetyl-CoA catalyzed by ATF1. The constitutively high expression of ATF1would not necessarily lead to high BA production. For example, BA production in FJ-008 in which atf1 was driven by the constitutive strong promoter Pₜₕₗ from C. tyrobutyricum was actually much lower (3.5 g/L vs 5.5 g/L) than in FJ-004 in which atf1 was expressed under the promoter P_{cat} from C. tyrobutyricum. We hypothesized that, in order to obtain more efficient BA production, the synthesis of ATF1 should be dynamically controlled and thus synchronous with the synthesis of precursors (butanol or acetyl-CoA). Therefore, for the next step, we attempted to evaluate various native promoters for atf1 expression, and identify the one(s) that can enable an appropriately dynamic expression of ATF1 and lead to enhanced BA production.

Four promoters associated with the synthesis of BA precursors were selected to drive the atf1 expression, and four strains were constructed correspondingly for BA production (FIG. 3C). Fermentation results were shown in FIG. 3D. Indeed, distinct results for BA production were observed in these strains. The BA level was only 1.0 and 1.1 g/L in FJ-305 and FJ-302 with P_{bdh} and P_{pfl} for atf1 expression, respectively. P_{ald} is an important promoter in C. saccharoperbutylacetonicum, which can sense the acidic state and switch cell metabolism from acidogenesis to solventogenesis³¹. FJ-303 with the atf1 expression driven by P_{ald} produced 9.9 g/L BA, which was still 20% lower than in FJ-301. Interestingly, FJ-304 in which atf1 was expressed under P_{adh} produced 13.7 g/L BA, which was about 10.5% higher than in FJ-301. Based on the results, we speculated that the promoter of adh (which encodes the key enzyme catalyzing butanol and ethanol production) might have resulted in the more appropriate dynamic expression of atf1 in line with the flux of the precursors and thus led to enhanced BA production. On the other hand, ethanol production in FJ-304 was also lower than in FJ-301 (0.3 g/L vs 0.6 g/L). The enhanced BA production in FJ-304 consumed more acetyl-CoA, and thus decreased production of ethanol, which also needed acetyl-CoA as the precursor.

### Organization of BA-Synthesis Enzymes to Enhance BA Production

In this study, we evaluated several reorganization approaches to increase BA production (FIG. 4).

PduA* protein, derived from Citrobacter freundii Pdu bacterial microcompartment could form filaments in bacteria like E. coli³². The CC-Di-A and CC-Di-B are designed parallel heterodimeric coiled coils and two proteins with each of these self-assembling tags could combine and shorten the catalytic distance. The enzymes (from the same metabolic pathway), tagged with one of the coiled coils (CC-Di-A or CC-Di-B) would attach onto the formed intracellular filaments (its PduA* was tagged by the other coiled coil); thus, the organized enzymes on the filaments would improve the catalytic efficiency of the target metabolic pathway. MinD is a membrane-associated protein and the localization of MinD is mediated by an 8-12 residue C-terminal membrane-targeting sequence. The proteins with MinD C-terminal sequence were able to be drawn to the cell membrane³²,³³. Thus, the application of MinD C-tag can facilitate the secretion of target product and enhance its production by mitigating the intracellular toxicity as well as promoting the catalyzing process (FIG. 4A).

To evaluate whether the organization of enzymes could improve BA production in our strain, three strategies were recruited: 1) assembling two of the three enzymes (enzymes associated with BA synthesis: NifJ (related to acetyl-CoA synthesis), BdhA (related to butanol synthesis) and ATF1) with the CC-Di-A and CC-Di-B tags; 2) organizing the three enzymes onto PduA* formed scaffold; or 3) introducing MinD C-tag to draw ATF1 onto the cell membrane.

Firstly, we assembled enzymes for BA synthesis by adding the CC-Di-A tag to ATF1 and the CC-Di-B tag to NifJ and BdhA (FIG. 4B). Fermentation results showed that the addition of CC-Di-A tag to the C-terminus of ATF1 in FJ-306 had significantly negative effects on BA synthesis with only 2.6 g/L BA was produced (FIG. 4E). The assembly of ATF1 together with NifJ or BdhA had even severer negative effects on BA synthesis and BA production was only 0.06 and 0.03 g/L in FJ-309 and FJ-310, respectively. Further, we organized ATF1, NifJ and BdhA onto the PduA* scaffold. PduA* was tagged with CC-Di-B, while the other three enzymes were tagged with CC-Di-A (FIG. 4C). The generated FJ-311 (harboring the scaffold and ATF1-CC-Di-A) produced 3.1 g/L BA, while FJ-312 (harboring the scaffold, ATF1-CC-Di-A and NifJ-CC-Di-A) and FJ-313 (harboring the scaffold, ATF1-CC-Di-A, NifJ-CC-Di-A and BdhA-CC-Di-A) produced slightly higher amount of BA both at 3.5 g/L. The scaffold seemed to have some positive effects on BA synthesis but didn’t work as efficient as it was reported in other studies³². We speculate that the coiled coils tags (CC-Di-A or CC-Di-B) might severely impair the catalytic activity of ATF1. Furthermore, the assembly of ATF1 together with NifJ or BdhA could further inhibit the activity of ATF1, thus resulting in significant decrease in BA production in the corresponding strains.

Moreover, we evaluated the effect of the introduction of MinD C-tag (to draw ATF1 onto the cell membrane) on BA production (FIG. 4D). Fermentation results showed that the corresponding FJ-308 strain could produce 16.4 g/L BA, which was 20% higher than FJ-304 (FIG. 4E). The FJ-307 strain (with CC-Di-A-Atf1-MinD) could also produce higher BA of 3.9 g/L compared to FJ-306 (2.6 g/L). All these results indicated that the addition of the cell membrane associated motif to draw ATF1 onto the cell membrane could facilitate the BA excretion and mitigate the intracellular toxicity and therefore enhance BA production. However, the assembly of BA synthetic enzymes or the organization of relevant enzymes onto the scaffold would significantly decrease BA production.

### Elimination of Prophages Increased BA (and BB) Production

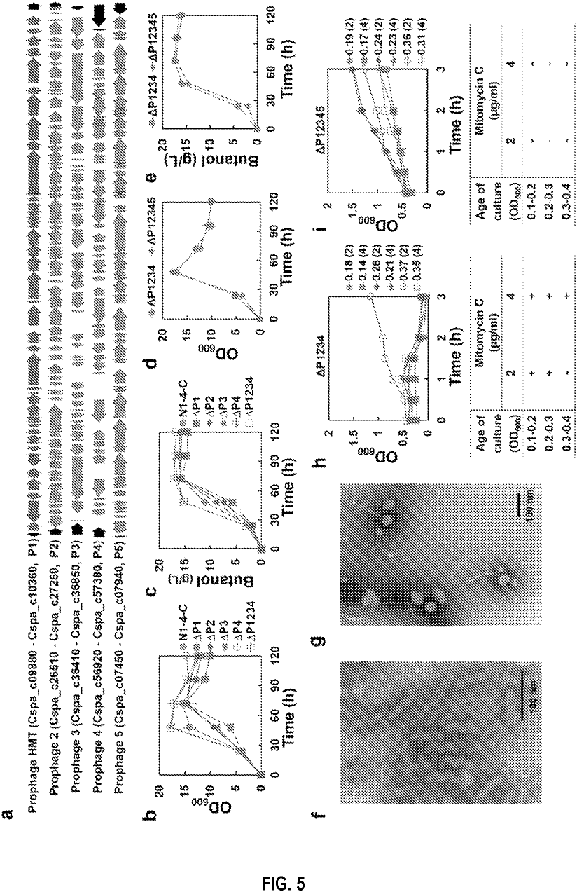

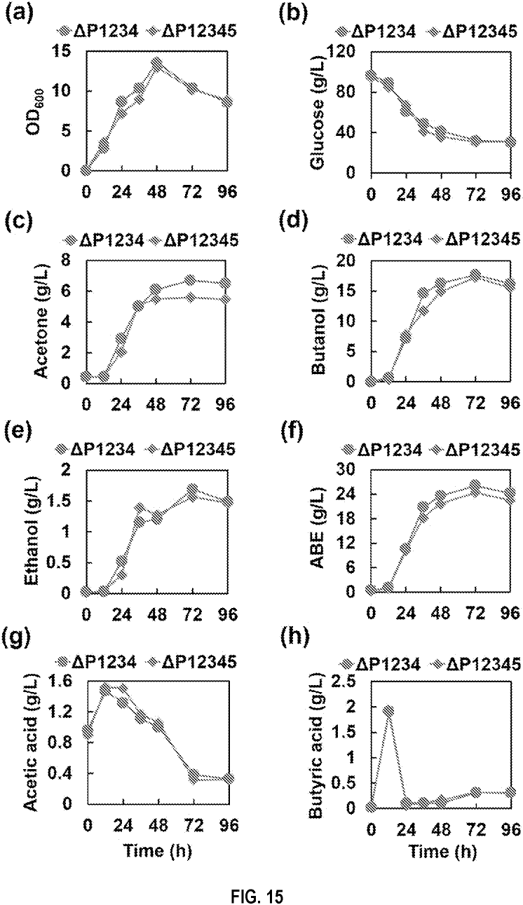

During our fermentations, we noticed that the ester production of the strains was not stable and could be varied from batch to batch. It has been reported that the N1-4 (HMT) strain contains a temperate phage named HM T which could release from the chromosome even without induction³⁴. In addition, the N1-4 (HMT) strain can produce a phage-like particle clostocin O upon the induction with mitomycin C³⁵. We hypothesized that the instability of fermentations with C. saccharoperbutylacetonicum might be related to the prophages, and the deletion of prophages would enable more stable and enhanced production of desired endproducts. We identified five prophage-like genomes (referred here as P1-P5 respectively) within the chromosome of N1-4 (HMT) (FIG. 5a)³⁶. Based on systematic evaluation through individual and combinatory deletion of the prophages, we demonstrated that P5 is responsible for the clostocin O synthesis (FIGS. 5f, h & 12)³⁵, and further confirmed that P1 was the HM T phage genome (FIGS. 5g & 14). However, the phage image was different from what was described before³⁷. It was more like HM 7 (a head with a long tail), rather than HM 1 (a head with multiple short tails). Both ΔP1234 (with the deletion of P1-P4) and ΔP12345 (with the deletion of P1-P5) exhibited improved cell growth and enhanced butanol production (FIGS. 5b-5e, FIGS. 10, 15 & 16). ΔP12345 should be a more stable platform as there is no cell lysis at any induction conditions with mitomycin C or norfloxacin (FIG. 5i). While ΔP1234 showed similar growth and even slightly higher butanol production compared to ΔP12345 (FIGS. 5d & 5e).

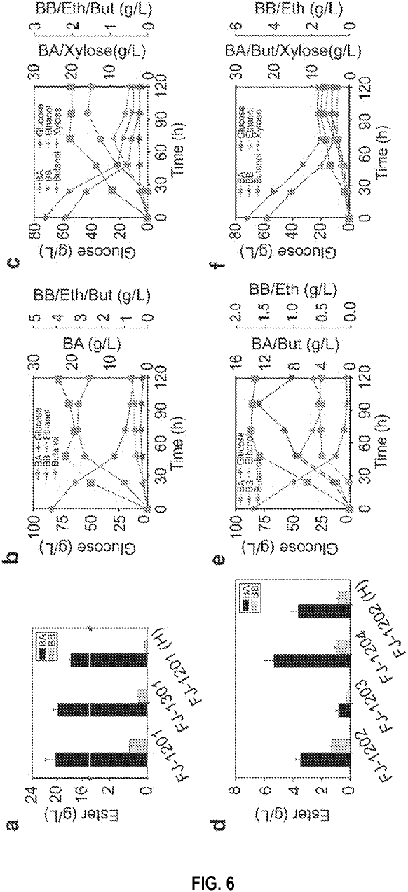

Thus, in a further step, we used ΔP1234 and ΔP12345 as the platform to be engineered for enhanced and more stable BA production. We deleted nouG and integrated sadh-hydG cluster in both ΔP1234 and ΔP12345, and obtained FJ-1200 and FJ-1300 correspondingly. The plasmid pMTL-P_{adh}-atf1-MinD was transformed into FJ-1200 and FJ-1300, generating FJ-1201 and FJ-1301, respectively. Fermentation results showed that FJ-1201 and FJ-1301 produced 19.7 g/L and 19.4 g/L BA, respectively, which were both higher than FJ-308 (FIG. 6a & Table S3). BA production in both FJ-1201 and FJ-1301 could be completed within 48 h, resulting in a productivity of ~0.41 g/L/h, which was significantly higher than 0.23 g/L/h in FJ-308. BA yield in FJ-1201 reached 0.26 g/g, which was also higher than FJ-308 (0.24 g/g). We further determined BA concentrations in the fermentation broth as 0.6 g/L and 0.5 g/L respectively for the fermentation with FJ-1201 and FJ-1301. Taken together, total BA production in FJ-1201 was 20.3 g/L, which was the highest level that has ever been reported in a microbial host. It is 2400-fold higher than the highest level that has been previously reported²², and also 14.8-fold higher than that by the very recently reported C. diolis strain²⁴.

Besides BA production, BB production in FJ-1201 also reached 0.9 g/L, which was significantly higher than in FJ-308 (0.01 g/L) and in C. pasteurianum J-5 (0.3 g/L) (FIG. 2b, FIG. 6a, & Table S3). This level (0.9 g/L) was 18.6-fold higher than the highest BB production that has been previously reported (0.05 g/L in C. acetobutylicum)²². All these results confirmed our hypothesis that the elimination of prophages would make more robust host strain for enhanced and more stable ester production.

### Expression of SAAT in FJ-1200 Further Enhanced BB Production

As demonstrated in FIG. 2b, SAAT and EHT1 were more relevant for BB production. Therefore, to achieve higher BB production, we expressed saat and eht1 in FJ-1200 and obtained FJ-1202 and FJ-1203, respectively. Fermentation showed that FJ-1202 and FJ-1203 produced 1.3 and 0.2 g/L BB, respectively (FIG. 6d). Further, we added MinD C-tag to the SAAT and introduced the recombinant gene into FJ-1200 and obtained FJ-1204 for an attempt to further improve BB production as observed for BA production in FJ-308. However, BB production in FJ-1204 was only 1.0 g/L. Notwithstanding, 1.3 g/L BB obtained in FJ-1202 is 25.8-fold higher than the highest level that has been previously reported²².

### Deletion of Rex Increased BA/BB Production

The rex gene was identified as a redox-sensing transcriptional repressor, which represses the transcription of genes related to butanol synthesis. The rex gene was deleted in FJ-1200, generating the strain YM016. Then the plasmid pMTL-Padh-atf1-MinD was transformed into YM016, obtaining the strain YM016P. The fermentation results indicated that 22.0 g/L BA could be produced in a batch fermentation with YM016P.

### Further Systematic Genome Engineering to Enhance BA Production

Based on YM016, three copies of ‘Padh-atf1-MinD’ cassette were integrated into different loci (ldh1, lacZ1 and lacZ2). Further, the ctfA1-ctfB1 gene cluster (encoding acetoacetyl-CoA:acetate/butyrate:CoA transferase) was deleted and the obtained strain was named YM028. Then, the plasmid pMTL-Padh-atf1-MinD was transformed into YM028, generating YM028P for BA production. Fermentation results showed that YM028P produced 25.3 g/L BA, which was the highest BA titer that we obtained by now.

### Ester Production With Biomass Hydrolysates as Substrate

Fermentations were carried out using biomass hydrolysates as the substrate. In the hydrolysates, besides sugars (57.4 g/L glucose and 27.2 g/L xylose) as carbon source, there were also nutrients converted from biomass (corn stover). Therefore, we tested the effect of organic nitrogen (yeast and tryptone) of various levels on ester production. Interestingly, results showed that the highest BA production of 17.5 g/L was achieved in FJ-1201 (in the extractant phase) without any exogenous nitrogen source supplemented (Table S4). In addition, 0.3 g/L BA was detected in aqueous phase, making a total BA production of 17.8 g/L in FJ-1201 (FIG. 6A & Table S4). Although this was slightly lower than when glucose was used as substrate (20.3 g/L), fermentation with hydrolysates did not need any supplementation of nutrients, which could significantly reduce production cost. We further performed fermentation in 500-mL bioreactor with pH controlled >5.0. BA production reached 16.0 g/L with hydrolysates and 18.0 g/L with glucose as substrate, both of which were lower than results from fermentation under the same conditions but with serum bottles (FIGS. 6B & 6C). During bioreactor fermentation, we noticed very strong smell of BA around the reactor. We suspected that significant evaporation during fermentation with bioreactor resulted in the lower level of final BA titers as compared to fermentation with serum bottle, which was securely sealed with only minimum outlet for releasing gases. An improved bioprocess needs to be carefully designed for larger scale fermentation to minimize BA evaporation and enhance BA production and recovery.

Furthermore, we performed fermentation with FJ-1202 for BB production using hydrolysates in both serum bottle and 500-mL bioreactor. Results demonstrated that BB production in serum bottle from hydrolysates was 0.9 g/L (compared to 1.3 g/L when glucose used as substrate; FIG. 6D). BB production in bioreactor from hydrolysates reached 0.9 g/L compared to 1.6 g/L when glucose was used as substrate (FIGS. 6E & 6F). The results were consistent with the case for BA production that lower-level BB was obtained when hydrolysates (compared to glucose) was used as substrate. However, interestingly, larger scale fermentation with bioreactor produced slightly higher level of BB than the fermentation under the same conditions with serum bottle, which was different from the case for BA production. This might be because BB is less evaporative (in bioreactor) than BA.

### Techno-Economic Analysis (TEA) for BA Production From Biomass Hydrolysates

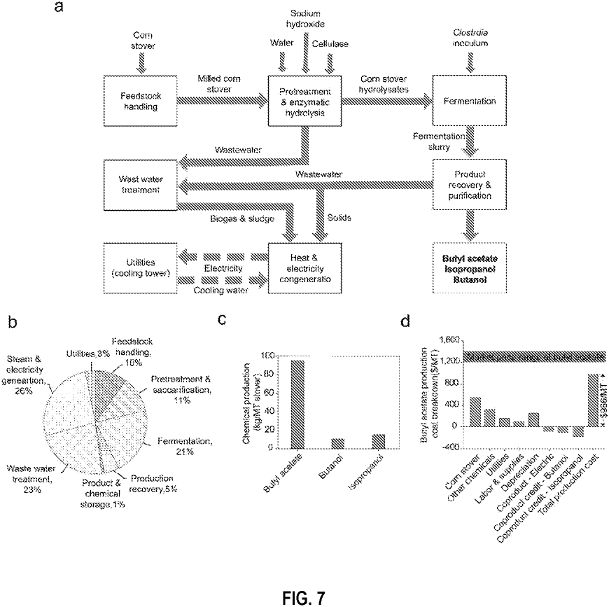

We performed a techno-economic analysis (TEA) to evaluate the economic competitiveness of BA production from corn stover at a process capacity of 2,500 MT wet corn stover (20% moisture) per day. The whole process was developed based on the previous process using the deacetylation and disk refining (DDR) pretreatment to produce corn stover hydrolysate³⁸, which was the substrate used for our fermentation experiments to produce BA. The detailed process information is summarized in the supplementary materials. The process is composed of eight sections including feedstock handling, DDR pretreatment and hydrolysis, BA fermentation, product recovery (distillation), wastewater treatment, steam and electricity generation, utilities, and chemical and product storage (FIG. 7A). FIG. 7B shows the equipment cost distribution of each process sections, with a total installed equipment cost of $263 million. The fermentation, steam & electricity cogeneration, and wastewater treatment contribute significant percentage to the total installed equipment cost, which aligns well with previous TEA models for chemical production from biomass via fermentation^{39,40}. The total capital investment (TCI) is $472 million by taking consideration of additional direct cost, indirect cost as well as working capitals (Table S5). From the process model, 95.2 kg of BA can be produced from 1 MT of corn stover, meanwhile significant amounts of butanol (11.1 kg), isopropanol (15.5 kg) and surplus electricity (209 kWh) are produced as coproducts (FIG. 7C). The BA production cost was estimated to be $986/MT (FIG. 7d), which is much lower than the current

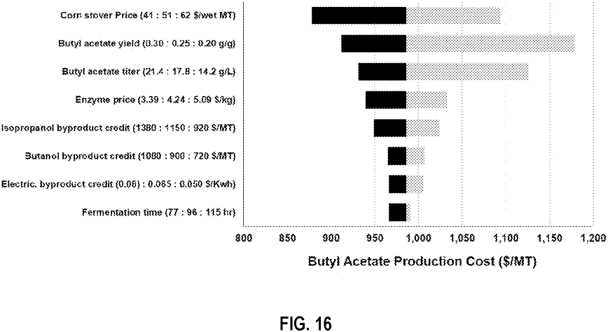

BA market price ranging between $1,200 and $1,400 per MT in year 2019 (based on the quotes from the industry⁴¹), showing the highly economic competitiveness of BA production using our engineered strain. By looking into the cost breakdown, the corn stover feedstock cost contributes the most (38.2%) to the BA production cost, followed by other chemicals (22.3%) and capital deprecation (18.0%) and utilities (14.5%). Sensitivity analysis shows that corn stover price, BA yield, and BA titer are the most sensitive input parameters to the BA production cost (FIG. 16).

### Discussion

Although tremendous efforts have been invested on biofuel/biochemical research worldwide, very limited success has been achieved. A key bottleneck is that the microbial host is subject to endproduct toxicity and thus desirable production efficiency cannot be obtained⁴². Our central hypothesis was that metabolically engineering of microorganisms for high-value and easy-recoverable bioproduct production can help alleviate end product toxicity and thus high titer and productivity can be achieved, with which economically viable biofuel/biochemical production can be ultimately established. Here we tested this hypothesis by engineering solventogenic clostridia for highly efficient ester (high-value and easy-recoverable) production. Based on the systematic screening of host strains and enzymes as well as multiple rounds of metabolic engineering: enriching precursors (alcohols and acetyl-CoA) for ester production, dynamically expressing heterologous ester-production pathways, organizing ester-synthesis enzymes, and improving strain robustness by eliminating putative prophages, we ultimately obtained strains for efficient production of esters in both synthetic fermentation medium and biomass hydrolysates. To the best of our knowledge, the production levels of BA and BB we achieved set up the new records.

### Methods

### Microorganisms and Cultivation Conditions

All the strains and plasmids used in this study are listed in Table S1. C. pasteurianum ATCC 6013 and C. saccharoperbutylacetonicum N1-4 (HMT) (DSM 14923) were requested from American Type Culture Collection (ATCC) and Deutsche Sammlung von Mikroorganismen und Zellkulturen (DSMZ), respectively. C. beijerinckii NCIMB 8052 was provided by Dr. Hans P. Blaschek¹⁴. C. tyrobutyricum Δcat1::adhE1 and C. tyrobutyricum Δcat1::adhE2 are hyper-butanol producing mutants constructed in our lab¹¹. All the clostridial strains were grown in an anaerobic chamber (N₂—CO₂—H₂ with a volume ratio of 85:10:5) at 35 ºC. Strains of C. tyrobutyricum, C. saccharoperbutylacetonicum and C. beijerinckii were cultivated using tryptone-glucose-yeast extract (TGY) medium⁴³, while strains of C. pasteurianum were cultivated using 2×YTG medium⁴⁴. When required, clarithromycin (Cla) or thiamphenicol (Tm) was supplemented into the medium at a final concentration of 30 µg/mL and 15 µg/mL, respectively. E._coli_DH5α was used for routine plasmid propagation and maintenance. E. coli CA434 was used as the donor strain for plasmid conjugation for C. tyrobutyricum. Strains of E. coli were grown aerobically at 37 ºC in Luria-Bertani (LB) medium supplemented with 100 µg/mL ampicillin (Amp), 50 µg/mL kanamycin (Kan) or 34 µg/mL chloramphenical (Cm) as needed.

### Plasmid Construction

All the plasmids used in this study are listed in Table S1, and all the primers used in this study are listed in Table S2.

The plasmids pMTL82151 and pTJ1 were used as mother vectors for heterogeneous gene expression^{45,46}. The promoter of the cat1 gene (CTK_C06520) (P_{cat}) and the promoter of the thl gene (CTK_C01450) (Pₜₕₗ) from C. tyrobutyricum ATCC 25755 were amplified and inserted into pMTL82151 at the EcoRI site, and the generated plasmids were named as pMTL82151-P_{cat} and pMTL82151-Pₜₕₗ, respectively. Promoters of the following gene, pflA (Cspa_c13710) (P_{pfl}), ald (Cspa_c56880) (P_{ald}), adh (Cspa_c04380) (P_{adh}) and bdh (Cspa_c56790) (P_{bdh}), all from C. saccharoperbutylacetonicum N1-4 (HMT) were amplified and inserted into pMTL82151 at the EcoRI site, and the generated plasmids were named as pMTL82151-P_{pfl}, pMTL82151-P_{ald}, pMTL82151-P_{adh} and pMTL82151-P_{bdh}, respectively.

The vaat gene from Fragaria vesca, the saat gene from F. ananassa and the atf1 gene from S. cerevisiae were amplified from plasmids pDL006, pDL001 and pDL004, respectively^{3,21}_ ENREF_26. The atf1′ (the codon optimized atf1 gene), eht1 from S. cerevisiae¹⁹, and lipaseB from Candida antarctica⁴⁷ were all synthesized by GenScript (Piscataway, NJ, USA). The obtained gene fragments of vaat, saat, atf1, atf1′, eth1, and lipaseB were inserted between the BtgZI and EcoRI sites in pMTL82151-P_{cat}, generating pMTL-P_{cat}-vaat, pMTL-P_{cat}-saat, pMTL-P_{cat}-atf1, pMTL-P_{cat}-atf1′, pMTL-P_{cat}-eht1, and pMTL-P_{cat}-lipaseB, respectively. The atf1 gene was inserted between the BtgZI and EcoRI sites in pMTL82151-Pₜₕₗ, generating pMTL-Pₜₕₗ-atf1.

The P_{cat} promoter and the gene fragments of vaat, saat, atf1, eth1, and lipase were amplified and ligated into the EcoRI site of pTJ1, generating pTJ1-P_{cat}-vaat, pTJ1-P_{cat}-saat, pTJ1-P_{cat}-atf1, pTJ1-P_{cat}-eht1 and pTJ1-P_{cat}-lipaseB, respectively. The atf1 gene was inserted into the EcoRI site of pMTL82151-P_{pfl}, pMTL82151-P_{ald}, pMTL82151-P_{adh} and pMTL82151-P_{bdh}, generating pMTL-P_{pfl}-atf1, pMTL-P_{ald}-atf1, pMTL-P_{adh}-atf1 and pMTL-P_{bdh}-aft1, respectively.

DNA sequences of CC-Di-A, CC-Di-B, MinD and pduA* were synthesized by GenScript (Piscataway, NJ, USA). The MinD-tag was fused to the end of atf1 with PCR and ligated into the EcoRI site of pMTL-P_{adh}, generating pMTL-P_{adh}-atf1-MinD. In addition, the MinD-tag was fused to the end of saat with PCR and inserted between the BtgZI and EcoRI sites of pMTL-P_{cat}, generating pMTL-P_{cat}-saat-MinD.

The synthesized CC-Di-A fragment was ligated into the EcoRI site of pMTL-P_{adh}, generating pMTL-P_{adh}-CC-Di-A. The DNA fragments of atf1 and atf1-MinD were amplified from pMTL-P_{adh}-atf1 and pMTL-Padh-atf1-MinD and then inserted into the EcoRI site of pMTL-P_{adh}-CC-Di-A, obtaining pMTL-P_{adh}-A-atf1 and pMTL-P_{adh}-A-atf1-MinD. The CC-Di-B sequence with the nifJ gene and CC-Di-B with the bdhA gene were subsequently inserted into the KpnI site of pMTL-P_{adh}-A-atf1, generating pMTL-P_{adh}-A-atf1-B-nifJ and pMTL-P_{adh}-A-atf1-B-nifJ-B-bdhA. The DNA fragments of CC-Di-B-pduA*, CC-Di-A-nifJ, CC-Di-A-bdhA were inserted into the EcoRI site of pTJ1-P_{cat}, generating pTJ1-P_{cat}-B-pduA*, pTJ1-P_{cat}-B-pduA*-A-nifJ and pTJ1-P_{cat}-B-pduA*-A-nifJ-A-bdhA, respectively.

For the gene deletion or integration in C. saccharoperbutylacetonicum, all the relevant plasmids were constructed based on pYW34, which carries the customized CRISPR-Cas9 system for genome editing in C. saccharoperbutylacetonicum^{14,27}. The promoter P_{J23119} and the gRNA (with 20-nt guide sequence targeting on the specific gene) were amplified by two rounds of PCR with primers N-20nt/YW1342 and YW1339/YW1342 as described previously (N represents the targeted gene)²⁷. The obtained fragment was then inserted into pYW34 (digested with BtgZI and NotI) through Gibson Assembly, generating the intermediate vectors. For gene deletion, the fragment containing the two corresponding homology arms (500-1000 bp for each) for deleting the specific gene through homologous recombination was amplified and inserted into the NotI site of the obtained intermediate vector as described above, generating pYW34-ΔN (N represents the targeted gene). For gene integration, the fragment containing the two corresponding homology arms (~1000-bp for each), the promoter and the gene fragment to be integrated, was amplified and inserted into the NotI site of the obtained intermediate vector as described above, generating the final plasmid for gene integration purpose.

### Fermentation With Glucose as the Substrate

For the fermentation for ester production, the C. pasteurianum strain was cultivated in Biebl medium⁴⁸ with 50 g/L glycerol as the carbon source at 35° C. in the anaerobic chamber. When the OD₆₀₀ reached ~0.8, the seed culture was inoculated at a ratio of 10% into 100 mL of the same medium in a 250-mL serum bottle and then cultivated at an agitation of 150 rpm and 30° C. (on a shaker incubator) for 72 h. The C. beijerinckii strain was cultivated in TGY medium until the OD₆₀₀ reached ~0.8. Then the seed culture was inoculated at a ratio of 5% into 100 mL P2 medium along with 60 g/L glucose and 1 g/L yeast extract in a 250-mL serum bottle. The fermentation was carried out at an agitation of 150 rpm and 37° C. for 72 h⁴³. The C. tyrobutyricum strain was cultivated in RCM medium at 35° C. until the OD₆₀₀ reached ~1.5. Then the seed culture was inoculated at a ratio of 5% into 200 mL fermentation medium (containing: 50 g/L glucose, 5 g/L yeast extract, 5 g/L tryptone, 3 g/L (NH₄)₂SO₄, 1.5 g/L K₂HPO₄, 0.6 g/L MgSO₄▪7H₂O, 0.03 g/L FeSO₄▪7H₂O, and 1 g/L L-cysteine) in a 500-mL bioreactor (GS-MFC, Shanghai Gu Xin biological technology Co., Shanghai, China) and the fermentation was carried out at an agitation of 150 rpm and 37° C. for 120 h with pH controlled >6.0⁹. The C. saccharoperbutylacetonicum strain was cultivated in TGY medium at 35° C. in the anaerobic chamber until the OD₆₀₀ reached ~0.8. Then the seed culture was inoculated at a ratio of 5% into 100 mL P2 medium along with 60 g/L glucose and 1 g/L yeast extract in a 250-mL serum bottle. The fermentation was carried out at an agitation of 150 rpm and 30° C. for 120 h⁴³. For the fermentation at larger scales in bioreactors, it was carried out in a 500-mL fermenter (GS-MFC, Shanghai Gu Xin biological technology Co., Shanghai, China) with a working volume of 250 mL with pH controlled >5.0, at 50 rpm and 30° C. for 120 h. Samples were taken every 24 h for analysis.

For all fermentations in the serum bottle, a needle and hosepipe were connected to the top of bottle for releasing the gases produced during the fermentation. For all the fermentations for ester production, the extractant n-hexadecane was added into the fermentation with a ratio of 1:1 (volume of the extractant vs. volume of fermentation broth) for in situ ester extraction. The reported ester concentrations were the determined values in the extractant phase. All the fermentations were carried out in triplicate.

### Fermentation With Biomass Hydrolysates as the Substrate

The biomass hydrolysates was kindly provided by Dr. Daniel Schell from National Renewable Energy Laboratory (NREL) which was generated from corn stover through the innovative ‘deacetylation and mechanical refining in a disc refiner (DDR)’ approach⁴⁹. For the fermentation, the biomass hydrolysate was diluted and supplemented into the P2 medium as the carbon source (with final sugar concentrations of 57.4 g/L glucose and 27.2 g/L xylose). In addition, various concentrations of yeast extract (Y, g/L) and tryptone (T, g/L) were also added as the nitrogen source to evaluate their effects on the fermentation performance: 0Y+0T; 1Y+3T and 2Y+6T. The fermentation was carried out under the same conditions as described above at 100 mL working volume in a 250-mL serum bottle. All the fermentations were carried out in triplicate.

### Analytical Methods

Concentrations of acetone, ethanol, butanol, acetic acid, butyric acid and glucose were measured using a high-performance liquid chromatography (HPLC, Agilent Technologies 1260 Infinity series, Santa Clara, CA) with a refractive index Detector (RID), equipped with an Aminex HPX-87H column (Bio-Rad Laboratories, Hercules, CA). The column was eluted with 5 mM H₂SO₄ at a flow rate of 0.6 mL/min at 25° C. The concentration of the ester in the n-hexadecane phase was quantified using a gas chromatography-mass spectrometry (GC-MS, Agilent Technologies 6890N, Santa Clara, CA) equipped with an HP-5 column (60 m×0.25 mm, 0.25 mm film thickness). Helium was used as the carrier gas. The initial temperature of the oven was set at 30° C. for 2 min, followed by a ramp of 10° C./min to reach 300° C., and a ramp of 2° C./min to reach the final temperature of 320° C., which was then held for 2 min. The detector was kept at 225.⁹

### REFERENCES

The following references are provided as an aid in understanding the subject matter provided above. No admission is made that any of the following meet the legal definition of “prior art” in any country, or that any of the following are relevant to the patentability of anything that is claimed.

1 Stephanopoulos, G. Challenges in engineering microbes for biofuels production. Science 315, 801-804 (2007).

2 Liao, J. C., Mi, L., Pontrelli, S. & Luo, S. Fuelling the future: microbial engineering for the production of sustainable biofuels. Nature Reviews Microbiology 14, 288 (2016).

3 Layton, D. S. & Trinh, C. T. Expanding the modular ester fermentative pathways for combinatorial biosynthesis of esters from volatile organic acids. Biotechnology and bioengineering 113, 1764-1776 (2016).

4 U.S. Esters Market Size, Share & Trends Analysis Report By Product (Fatty Esters, Phosphate Esters, Acrylic Esters, Cellulose Esters, Allyl and Aromatic Esters), By Application, And Segment Forecasts, 2019-2025. (https: //www.grandviewresearch.com/industry-analysis/us-esters-market). Grand view reserach (2019).

5 Rodriguez, G. M., Tashiro, Y. & Atsumi, S. Expanding ester biosynthesis in Escherichia coli. Nature chemical biology 10, 259-265 (2014).

6 Liu, Y., Lotero, E. & Goodwin, J. G. Effect of water on sulfuric acid catalyzed esterification. Journal of Molecular Catalysis A: Chemical 245, 132-140 (2006).

7 van den Berg, C., Heeres, A. S., van der Wielen, L. A. & Straathof, A. J. Simultaneous clostridial fermentation, lipase-catalyzed esterification, and ester extraction to enrich diesel with butyl butyrate. Biotechnology and bioengineering 110, 137-142 (2013).

8 Stergiou, P.-Y. et al. Advances in lipase-catalyzed esterification reactions. Biotechnology advances 31, 1846-1859 (2013).

9 Zhang, Z. T., Taylor, S. & Wang, Y. In situ esterification and extractive fermentation for butyl butyrate production with Clostridium tyrobutyricum. Biotechnology and Bioengineering 114, 1428-1437 (2017).

10 Verstrepen, K. J. et al. Expression levels of the yeast alcohol acetyltransferase genes ATF1, Lg-ATF1, and ATF2 control the formation of a broad range of volatile esters. Applied and environmental microbiology 69, 5228-5237 (2003).

11 Zhang, J., Zong, W., Hong, W., Zhang, Z.-T. & Wang, Y. Exploiting endogenous CRISPR-Cas system for multiplex genome editing in Clostridium tyrobutyricum and engineer the strain for high-level butanol production. Metabolic engineering (2018).

12 Pyne, M. E., Bruder, M. R., Moo-Young, M., Chung, D. A. & Chou, C. P. Harnessing heterologous and endogenous CRISPR-Cas machineries for efficient markerless genome editing in Clostridium. Sci Rep 6, 25666, doi:10.1038/srep25666 (2016).

13 Gu, Y. et al. Curing the endogenous megaplasmid in Clostridium saccharoperbutylacetonicum N1-4 (HMT) using CRISPR-Cas9 and preliminary investigation of the role of the plasmid for the strain metabolism. Fuel 236, 1559-1566 (2019).

14 Wang, Y. et al. Bacterial genome editing with CRISPR-Cas9: deletion, Integration, single nucleotide modification, and desirable “clean” mutant selection in Clostridium beijerinckii as an example. ACS synthetic biology 5, 721-732 (2016).

15 Yoo, M., Croux, C., Meynial-Salles, I. & Soucaille, P. Elucidation of the roles of adhE1 and adhE2 in the primary metabolism of Clostridium acetobutylicum by combining in-frame gene deletion and a quantitative system-scale approach. Biotechnol Biofuels 9, 92, doi:10.1186/s13068-016-0507-0 (2016).

16 Han, S.-Y. et al. Highly efficient synthesis of ethyl hexanoate catalyzed by CALB-displaying Saccharomyces cerevisiae whole-cells in non-aqueous phase. Journal of Molecular Catalysis B: Enzymatic 59, 168-172 (2009).

17 Aharoni, A. et al. Identification of the SAAT gene involved in strawberry flavor biogenesis by use of DNA microarrays. The Plant Cell 12, 647-661 (2000).

18 Beekwilder, J. et al. Functional characterization of enzymes forming volatile esters from strawberry and banana. Plant Physiology 135, 1865-1878 (2004).

19 Saerens, S. M. et al. The Saccharomyces cerevisiae EHT1 and EEB1 genes encode novel enzymes with medium-chain fatty acid ethyl ester synthesis and hydrolysis capacity. Journal of Biological Chemistry 281, 4446-4456 (2006).

20 Tai, Y.-S., Xiong, M. & Zhang, K. Engineered biosynthesis of medium-chain esters in Escherichia coli. Metabolic engineering 27, 20-28 (2015).

21 Layton, D. S. & Trinh, C. T. Engineering modular ester fermentative pathways in Escherichia coli. Metabolic engineering 26, 77-88 (2014).

22 Noh, H. J., Woo, J. E., Lee, S. Y. & Jang, Y.-S. Metabolic engineering of Clostridium acetobutylicum for the production of butyl butyrate. Applied microbiology and biotechnology, 1-9 (2018).

23 Alonso-Gutierrez, J. et al. Metabolic engineering of Escherichia coli for limonene and perillyl alcohol production. Metabolic engineering 19, 33-41 (2013).

24 Li, A. et al. Developing Clostridium diolis as a biorefinery chassis by genetic manipulation. Bioresour Technol 305, 123066, doi:10.1016/j.biortech.2020.123066 (2020).

25 Yagi, T., Yano, T., Di Bernardo, S. & Matsuno-Yagi, A. Procaryotic complex I (NDH-1), an overview. Biochimica et Biophysica Acta (BBA)-Bioenergetics 1364, 125-133 (1998).

26 Liu, J. et al. Enhanced butanol production by increasing NADH and ATP levels in Clostridium beijerinckii NCIMB 8052 by insertional inactivation of Cbei_4110. Applied microbiology and biotechnology 100, 4985-4996 (2016).

27 Wang, S., Dong, S., Wang, P., Tao, Y. & Wang, Y. Genome Editing in Clostridium saccharoperbutylacetonicum N1-4 with the CRISPR-Cas9 System. Applied and Environmental Microbiology 83, e00233-00217 (2017).

28 Hong, R. The cloning of a putative regulatory gene and the sol region from Clostridium beijerinckii, Virginia Tech, (1999).

29 Jang, Y. S. et al. Metabolic engineering of Clostridium acetobutylicum for the enhanced production of isopropanol-butanol-ethanol fuel mixture. Biotechnology progress 29, 1083-1088 (2013).

30 Wang, P., Feng, J., Guo, L., Fasina, O. & Wang, Y. Engineering Clostridium saccharoperbutylacetonicum for high level Isopropanol-Butanol-Ethanol (IBE) production from acetic acid pretreated switchgrass using the CRISPR-Cas9 system. ACS Sustainable Chemistry & Engineering 7, 18153-18164 (2019).

31 Kosaka, T., Nakayama, S., Nakaya, K., Yoshino, S. & Furukawa, K. Characterization of the sol operon in butanol-hyperproducing Clostridium saccharoperbutylacetonicum strain N1-4 and its degeneration mechanism. Bioscience, biotechnology, and biochemistry 71, 58-68 (2007).

32 Lee, M. J. et al. Engineered synthetic scaffolds for organizing proteins within the bacterial cytoplasm. Nature chemical biology (2017).

33 Szeto, T. H., Rowland, S. L., Habrukowich, C. L. & King, G. F. The MinD membrane targeting sequence is a transplantable lipid-binding helix. Journal of Biological Chemistry 278, 40050-40056 (2003).

34 Hongo, M., Murata, A. & Ogata, S. Bacteriophages of Clostridium saccharoperbutylacetonicum: Part XVI. Isolation and some characters of a temperate phage. Agricultural and Biological Chemistry 33, 337-342 (1969).

35 Ogata, S., Mihara, O., Ikeda, Y. & Hongo, M. Inducible phage tail-like particles of Clostridium saccharoperbutylacetonicum and its related strains. Agricultural and Biological Chemistry 36, 1413-1421 (1972).

36 Zhou, Y., Liang, Y., Lynch, K. H., Dennis, J. J. & Wishart, D. S. PHAST: a fast phage search tool. Nucleic acids research 39, W347-W352 (2011).

37 Ogata, S., Nagao, N., Hidaka, Z. & Hongo, M. Bacteriophages of Clostridium saccharoperbutylacetonicum: Part XVII. The structure of phage HM 2. Agricultural and Biological Chemistry 33, 1541-1552 (1969).

38 D. Humbird, R. D., L. Tao, C. Kinchin,, D. Hsu, A. A., P. Schoen, J. Lukas, B. Olthof, M. Worley, & D. Sexton, a. D. D. Process design and economics for biochemical conversion of lignocellulosic biomass to ethanol:dilute-acid pretreatment and enzymatic hydrolysis of corn stover. (No. NREL/TP-5100-47764). National Renewable Energy Lab.(NREL), Golden, CO (United States). (2011).

39 Tao, L. et al. Techno-economic analysis and life-cycle assessment of cellulosic isobutanol and comparison with cellulosic ethanol and n-butanol. Biofuels, Bioproducts and Biorefining 8, 30-48 (2014).

40 Huang, H., Long, S. & Singh, V. Techno-economic analysis of biodiesel and ethanol coproduction from lipid-producing sugarcane. Biofuels, Bioproducts and Biorefining 10, 299-315 (2016).

41 https://www.alibaba.com/product-detail/Purity-99-5-Butyl-Acetate-Cas_62416155030.html?spm=a2700.galleryofferlist.0.0.67372e613qq3zW&s=p

42 Gong, Z., Nielsen, J. & Zhou, Y. J. Engineering robustness of microbial cell factories. Biotechnology journal 12, 1700014 (2017).

43 Wang, Y. et al. Development of a gene knockout system using mobile group II introns (Targetron) and genetic disruption of acid production pathways in Clostridium beijerinckii. Applied and environmental microbiology 79, 5853-5863 (2013).

44 Schwarz, K. M. et al. Towards improved butanol production through targeted genetic modification of Clostridium pasteurianum. Metabolic engineering 40, 124-137 (2017).

45 Heap, J. T., Pennington, O. J., Cartman, S. T. & Minton, N. P. A modular system for Clostridium shuttle plasmids. Journal of microbiological methods 78, 79-85 (2009).

46 Wang, Y. et al. Development of a gene knockout system using mobile group II introns (Targetron) and genetic disruption of acid production pathways in Clostridium beijerinckii. Applied and environmental microbiology, 79, 5853-5863 (2013).

47 Tamalampudi, S. et al. Development of recombinant Aspergillus oryzae whole-cell biocatalyst expressing lipase-encoding gene from Candida antarctica. Applied microbiology and biotechnology 75, 387 (2007).

48 Biebl, H. Fermentation of glycerol by Clostridium pasteurianum-batch and continuous culture studies. Journal of industrial microbiology & biotechnology 27, 18-26 (2001).

49 Chen, X. et al. A highly efficient dilute alkali deacetylation and mechanical (disc) refining process for the conversion of renewable biomass to lower cost sugars. Biotechnology for Biofuels 7, 98 (2014).

### SUPPLEMENTAL MATERIALS TO EXAMPLE

### Methods

### Plasmid Transformation and Mutant Verification

Competent cells of C. pasteurianum were prepared following the protocol as reported by Pyne et al¹. C. pasteurianum SD-1, an equivalent to C. pasteurianum ΔcpaAIR in which the cpaAIR gene (encoding the CpaAI Type II restriction endonuclease) was deleted and thus more efficient transformation was enabled², was used as the host strain. The overnight-grown seed culture was inoculated into 20 mL 2×YTG medium. When the OD₆₀₀ reached 0.3~0.4, sucrose and glycine were added to a final concentration of 0.4 M and 1.25%, respectively. When the OD₆₀₀ further reached 0.6~0.8, the cells were harvested by centrifugation at 4,200 g and 4° C. for 10 min. The cell pellets were resuspended in 5 mL of SMP buffer (270 mM sucrose, 1 mM MgCl₂ and 7 mM sodium phosphate, pH 6.5) and spinned down under the same conditions. The obtained cell pellets were then resuspended in 0.6 mL of SMP buffer. 1 µg of plasmid DNA was suspended with 20 µL of 2 mM Tris-HCl (pH 8.0) and then mixed with 550 µL competent cells and 30 µL 96% cold ethanol. The mixture was transferred to a pre-chilled 4 mm electroporation cuvette and incubated on ice for 5 min. Electroporation was then applied with a voltage of 1,800 V, capacitance of 25 µF and resistance of ∞ using a Gene Pulser Xcell electroporation system (Bio-Rad Laboratories, Hercules, CA). Afterwards, the culture was transferred into 2 mL of 2×YTG medium and recovered at 35° C. for 4 h. The culture was then spread onto the 2×YTGT agar plates (2×YTG agar plates containing 15 µg/mL of thiamphenicol) for the selection of the transformants.

Competent cells of C. beijerinckii were prepared following the procedure as described by Wang et al.³. Briefly, the overnight cell culture was inoculated into TGY medium with an inoculation ratio of 1%. When the OD₆₀₀ reached ~0.8, the cells were harvested by centrifugation at 4,200 g and 4° C. for 10 min. The cell pellets were washed with the same volume (as the original cell culture) of ice-cold 15% glycerol and centrifugated under the same condition for 10 min. The cell pellets were resuspended with 5% volume of ice-cold 15% glycerol. Then 400 µL competent cells and ~1.0 µg of plasmid DNA were mixed and transferred into a 2-mm pre-chilled electroporation cuvette and incubated on ice for 10 min. Electroporation was carried out at 2,000 V of voltage, 25 µF of capacitance and 200 Ω of resistance. Afterwards, the cells were transferred into 1.6 mL of TGY and incubated at 35° C. for 6-8 h for recovery. The culture was then spread onto TGYC agar plates (TGY agar plates containing 30 µg/mL of clarithromycin) for the selection of transformants.

The plasmid transformation through conjugation for C. tyrobutyricum was performed following the procedure as described by Zhang et al.⁴. The donor strain E. coli CA434 carrying the desired plasmid was cultivated in LB medium supplemented with Cm and Kan. 3 mL of overnight-cultured E. coli CA434 cells were centrifuged and washed for twice (with fresh LB medium) to remove the antibiotics. The obtained donor cells were then mixed with 0.4 mL of the overnight-cultured C. tyrobutyricum (grown in TGY medium). The cell mixture was spotted onto the TGY agar plate and incubated in the anaerobic chamber at 37° C. for conjugation. After 24 h of cultivation, the cell lawn on the plate was washed off using 1 mL of TGY medium and then spread onto the TGY plate containing 15 µg/mLTm and 250 µg/mL D-cycloserine (for eliminating the residual E. coli CA434 donor cells). The transformant colonies could be observed after 48-72 h of incubation in the anaerobic chamber.

Competent cells of C. saccharoperbutylacetonicum were prepared following the procedure as described by Herman et al., with slight modifications⁵. Briefly, the overnight-cultured cells were inoculated into fresh TGY medium. When the OD₆₀₀ of the cell culture reached ~0.8, cells were collected by centrifugation at 4,200 g and 22° C. (room temperature) for 10 min. Cell pellets were then washed with the same volume (as the original cell culture) of SMP buffer. The resuspension was centrifuged again under the same conditions as described above. The cell pellets were resuspended in 5% volume of SMP buffer. After that, the plasmid (~1.0 µg) was mixed with 400 µL of competent cells and transferred into a 2 mm electroporation cuvette and incubated on ice for 30 min. Electroporation was then applied with a voltage of 1,000 V, capacitance of 25 µF and resistance of 300 Ω. Subsequently, the culture was transferred into 2 mL pre-warmed TGY medium and incubated at 35° C. for 2-3 h. After that, the culture was spread onto TGYC or TGYT (TGY agar plates containing 15 µg/mL of thiamphenicol) agar plates.

The positive mutants of C. saccharoperbutylacetonicum with desired gene deletion or integration were identified following our previously described procedures⁶. Briefly, the C. saccharoperbutylacetonicum transformants harboring the plasmid designed for the gene deletion or integration were incubated in TGYC liquid medium at 35° C. in the anaerobic chamber for about 24 h. The cell culture was then spread onto TGYLC plates (TGYC supplemented with 40 mM lactose). When colonies appeared on the plates, colony PCR (cPCR) was then carried out with the pair of primers of N-U/N-D (N represents the targeted gene name, U represents the upstream primer flanking the target locus, and D represents the downstream primer flanking the target locus) to verify the gene deletion or gene integration. The selected mutants were then subcultured in TGY medium for 3 to 5 generations to cure the plasmid for the gene deletion/integrations⁶. The obtained plasmid-free and marker-free mutant strains were used for the following steps.

### Prophage Induction and Phage Harvesting

The strain was grown in TGY medium for overnight. The cells were then transferred into fresh TGY prior to be expose to the inducing reagent mitomycin C or norfloxacin. Mitomycin C was added into the culture when the cell growth reached the desired OD₆₀₀ (0.1-0.2, 0.2-0.3, 0.3-0.4 or 0.4-0.5). The cells of some of the cultures grew too fast at the early stage for the induction purpose, and thus they were not induced under every above mentioned OD₆₀₀ conditions in this work. For the induction, generally mitomycin C at final concentrations of 2 µg/mL and 4 µg/mL was used⁷. Besides, 1 µg/mL and 3 µg/mL of mitomycin C were also tried for the induction in ΔP234. After 30 min of treatment at 35° C., the cells were harvested via centrifugation at 4,000 g for 5 min and resuspended at the same volume of TGY fresh medium. Then the OD₆₀₀ was monitored carefully during the following 3-5 h.

The seed culture with the OD₆₀₀ of around 0.2, 0.4 and 0.6 was treated using norfloxacin at the final concentrations of 0.3, 1, 3, 6, 9 3 µg/mL at 35° C. for 24 h⁸. The final OD₆₀₀ was measured after the treatment.

The cells were collected via centrifugation at 4,000 g for 20 min, and then filtered through the 0.2 µm filter to obtain the supernatants. The supernatants were centrifugated at 20,000 rpm for 3 h, and the obtained precipitation (containing the phages) was then resuspended by 1/30 volume of ddH₂O.

### Transmission Election Microscopy (TEM)

20 µL prepared phage sample was applied to a mesh copper grid and settled for 2 min. Then the liquid was blotted off with a filter paper. The negative stain of 2% phosphotungstic acid (PTA) was applied to a mesh copper grid and settled for 30 s. Then the liquid was blotted off with a filter paper followed by air dry. The prepared samples were then used for TEM observation under a Zeiss EM10 transmission electron microscope (Carl Zeiss AG, Oberkochen, Germany) at an accelerating voltage of 60 kV.

### Techno-Economic Analysis (TEA)

A comprehensive TEA model was developed to evaluate the economic feasibility of BA production from corn stover using the deacetylation and disk refining (DDR) pretreatment. The model originally developed to produce ethanol⁹ was modified to produce BA by mainly substituting the fermentation and distillation unit operations. The processing capacity was set at 2,500 wet metric tonnes (MT, 20% moisture) of corn stover per day. The process was assumed to run 350 days (8,410 hours) per year; thus the annual corn stover consumption is 875,000 wet MT per year. The composition of corn stover was 35.05% cellulose, 19.53% hemicellulose, 15.76% lignin, 4.93% ash, 3.10% of protein, and 21.63% other solids on a dry basis¹⁰. All process was simulated using the software SuperPro Designer (Intelligen Inc., NJ).

The whole process can be divided into eight sections, including feedstock handling, DDR pretreatment and hydrolysis, fermentation, product recovery (distillation), wastewater treatment, steam and electricity cogeneration, utilities, and chemical and product storage. In the process, corn stover is milled at the pre-processing plant and delivered to the feed handling section from a uniform corn stover supply system¹⁰ Received corn stover is added with water to obtain a 25% solid slurry. The slurry is added with sodium hydroxide at a loading of 40 kg/MT dry corn stover, heated to 80° C. and held for 2 hours to remove acetyl groups from corn stover. The alkali-treated corn stover is then washed by using the same amount of added water, followed by dewatering using screw-type presses to remove excess water to attain 40% solids content for the subsequent disk refining and enzymatic hydrolysis. The parameters of disk refining are adapted from a previous optimization study using wet disk mills⁹.The electricity consumption of disk milling is 212 kWh per dry MT of corn stover. The details of the DDR process are described in Chen et al. 2015⁹. The pretreated corn stover is then cooled and sent to hydrolysis tanks where it is hydrolyzed into monomeric sugars by cellulase at 48° C. for 84 hr. The enzyme loading for the enzymatic hydrolysis is 19 mg protein/g cellulose according to a previous study⁹. After the enzymatic hydrolysis, 10% of the hydrolysate is split off to seed fermenters for production of seed culture, and the rest 90% of the hydrolysates is fermented into BA and coproducts in large fermenters at 32° C., where hexadecane is added to the fermenter at 1:1 ratio (v/v) to extract BA from the fermentation broth. The fermentation takes 96 hours to convert all hydrolyzed sugars to BA and coproducts butanol and isopropanol. The key parameters for the conversion and fermentation yields are summarized in Table S6. The fermentation beer is then sent to the decanter to separate the extractant phase and the aqueous phase, which are separately pumped to the distillation section to recovery BA and coproducts butanol and isopropanol. The distillation stillage is press filtered to separate insoluble solids for combustion to produce steam and electricity, whereas the pressed filtrate is sent to the waste treatment section. The remaining sections of the model, including wastewater treatment, steam and electricity cogeneration, and utilities, inherited most of the original designs from the NREL process model^{9, 10}.

The energy and mass balance and flow rate information for the process were generated to determine the capital and operating costs. The purchased equipment costs were determined based on previous literature, particularly from Humbird et al. (2011)¹⁰ and Chen et al. (2015)⁹. The cost of the product recovery (distillation) section was mainly determined by the embedded cost estimator of SuperPro Designer. The purchased equipment costs were scaled using the exponential scaling equation with exponents ranged between 0.5 and 0.8 depending on the type of equipment¹⁰. The equipment cost obtained in previous years is adjusted to the year of 2019 using the plant cost index from chemical engineering magazine. The total capital investment (TCI) was calculated as a sum of direct and indirect costs, which were determined based on the installed equipment costs. Direct costs included installed equipment cost, site development (9% of inside-battery-limits (ISBL) equipment cost), warehouse (4.0% of ISBL), and additional piping (4.5% of ISBL). Indirect cost is the sum of prorateable costs (10% of total direct cost (TDC)), field expenses (10% of TDC), home office and construction (20% of TDC), project contingency (10% of TDC), and other costs (10% of TDC). Working capital was assumed to be 5% of the fixed capital investment. A summary of the total operating cost is listed in Table S5. The total operating cost included both fixed and variable operating costs. Fixed operating costs include labor and various overhead items and variable operating costs include raw material costs, utility costs, and co-product credits. The detailed variable and fixed operating costs are summarized in Table S7.

The BA production cost was calculated based on the methods described in prior studies^{11,12}. For the process model, BA was assigned as the main product and butanol, isopropanol, and electricity were assigned as the coproducts, which were sold to generate coproduct credits. Sensitivity analyses were also performed at ± 20% variation range to evaluate the most influential variables on the BA production cost.

### Results

### Evaluation of Codon-Optimization of Atf1 for BA Synthesis

Different microorganisms have different codon usage preferences. The atf1 gene was originally from S. cerevisiae and its genetic codon usage might not be preferable for the C. saccharoperbutylacetonicum host. Thus, a codon optimized atf1 gene (designated as atf′) was synthesized and evaluated for potentially improved expression and BA production in the C. saccharoperbutylacetonicum host strain. However, fermentation results demonstrated that FJ-007 (carrying atf1′ rather than atf1) actually generated slightly lower concentration of BA than FJ-004 (5.0 g/L vs. 5.5 g/L, Table S8). The result was unexpected, but not totally surprising. Similar cases have been reported previously where the original natural gene showed better efficiency than the codon-optimized counterpart for desirable biochemical production¹³. We speculate that the natural gene might be able to transcribe into more stable mRNA structure, and thus lead to higher translation level than the codon-optimized gene¹³. In addition, it has been recently reported that codon-optimized genes could bring about toxicity to the host cells¹⁴.

### Enhancement of Acetyl-CoA Availability to Improve BA Production

In the pathway, thiolase is the enzyme that coverts acetyl-CoA into acetoacetyl-CoA (FIG. 3a), and the attenuation of the thiolase activity might be able to repress the acetyl-CoA flux towards butanol (and other products) and lead to the accumulation of intracellular acetyl-CoA, which could be beneficial for enhanced BA production. Therefore, we attempted to delete the thiolase gene to further increase the acetyl-CoA availability. There are five annotated genes encoding thiolase in C. saccharoperbutylacetonicum: Cspa_c06180, Cspa_c17310, Cspa_c20520, Cspa_c20890, Cspa_c50320. After numerous attempts, we were only able to delete Cspa_c20890 and Cspa_c50320 individually in FJ-300, generating the mutant strains FJ-400 and FJ-500, respectively. By introducing the vector pMTL-cat-atf1 into these two strains for BA production, FJ-401 and FJ-501 were obtained, respectively. Fermentation results indicated that BA production was actually slightly lower in FJ-401 (10.8 g/L) and FJ-501 (9.8 g/L) than that in FJ-301 (FIG. 3b). The result was unexpected. Acetoacetyl-CoA is one of the key metabolites for the cell; the repression for its biosynthesis might impair the cell metabolism and thus lead to decreased BA production.

### The Elimination of Prophages Increased Cell Growth and Butanol Production

During our fermentation process, we noticed that the performance for ester production of the strains was not very stable and could be varied from batch to batch. Our industrial collaborator also observed that C. saccharoperbutylacetonicum often had instable performance for ABE production in the continuous fermentation process (data not shown). It has been previously reported that the N1-4 (HMT) strain contains a temperate phage named HM T which could release from the chromosome even without induction¹⁵. In addition, the N1-4 (HMT) strain can produce a phage-like particle clostocin O with the induction of mitomycin C. We hypothesized that the instability of the fermentation with C. saccharoperbutylacetonicum might be related to the existence of prophages, and the deletion of these prophages and clostocin O encoding sequences would improve the stability of the strain and thus enable more stable and enhanced production of the desired endproduct (BA here). The online program PHAST was used to predict the prophage sequences in N1-4 (HMT)¹⁶ with four possible prophage genomes were identified: the HM T prophage (renamed as P1) as well as three other putative prophages which were named as P2, P3 and P4 here (FIG. 5a). Besides, one additional incomplete prophage genome (without integrase gene) was found and named as P5.

We firstly constructed the mutant with single deletion of each prophage genome and generated the ΔP1, ΔP2, ΔP3 and ΔP4 strains. Because there are genes within the prophage genome possibly responsible for the normal cell metabolism, we also constructed the mutant with the deletion of only the integrase gene (without the integrase, the prophage cannot release from the chromosome), obtaining the ΔNP1, ΔNP2, ΔNP3 and ΔNP4 strains. In addition, we also constructed the mutant ΔP1234 (with the deletion of all four prophage genomes) and ΔNP1234 (with the deletion of all four integrase genes).

Fermentations were first conducted in the serum bottle to investigate the effects of the elimination of prophages on butanol production in the mutant strains. As shown in FIGS. 5b, 5c & S3, ΔP4 and ΔP1234 showed increased cell growth and butanol production, while all the other eight mutants did not demonstrate significance difference in terms of the cell growth and solvent production compared to the mother strain. ΔP4 and ΔP1234 reached the maximum OD₆₀₀ of 17.9 and 17.6, which were 15.5% and 13.5% higher than that of the control N1-4-C strain, respectively. The butanol production in ΔP4 and ΔP1234 reached 17.1 and 16.8 g/L respectively, which were also higher than that of the control N1-4-C strain (16.0 g/L).

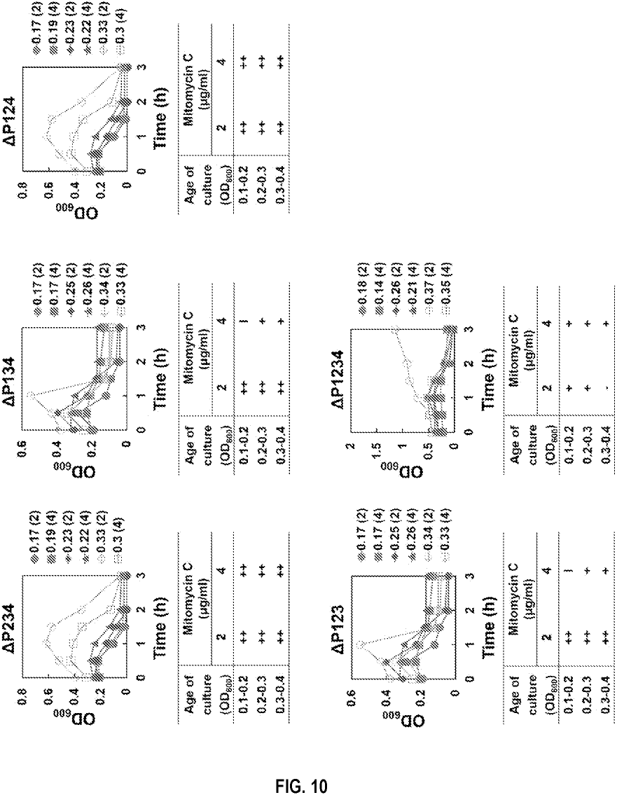

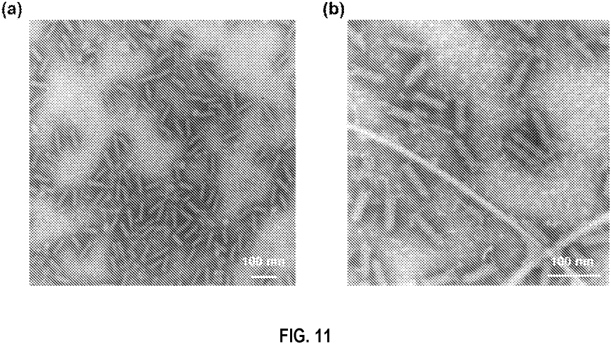

To further study the individual prophage, we constructed the triple-deletion mutants ΔP234, ΔP134, ΔP124, ΔP123. Phage induction experiments of ΔP234, ΔP134, ΔP124, ΔP123 and ΔP1234 with mitomycin C revealed that all the mutants exhibited cell lysis (FIG. 10). Transmission election microscopy (TEM) results indicated that all the mutants produced the tail-like particles, which showed likely the same appearance as clostocin O as reported previously⁷ (FIG. 11). We were not able to observe the HM T phage in the supernatant of ΔP234. It might be because there were too many clostocin O particles in the view which made it difficult to observe the HM T phage.

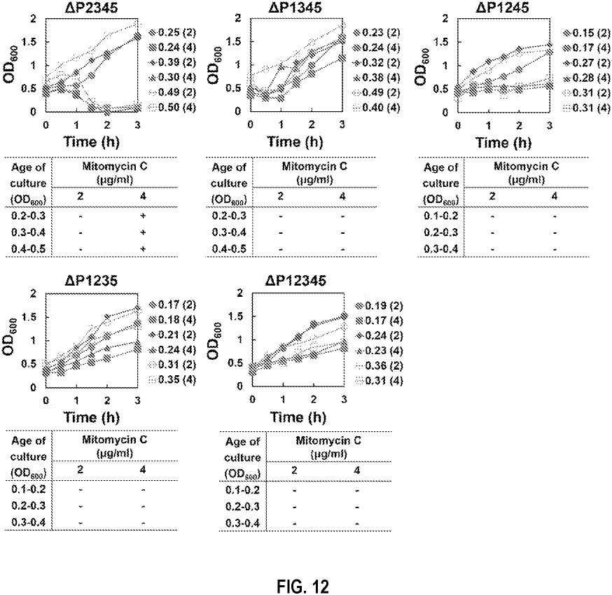

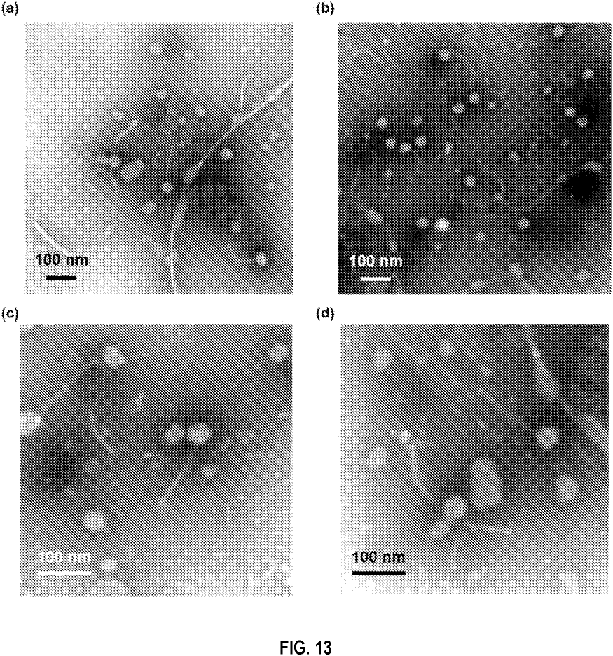

Based on the above results, we tentatively concluded that P5 might be responsible for the production of clostocin O. To verify this hypothesis and obtain a more robust strain for bioproduction, P5 was deleted in ΔP234, ΔP134, ΔP124, ΔP123 and ΔP1234, obtaining ΔP2345, ΔP1345, ΔP1245, ΔP1235 and ΔP12345. The induction experiments indicated that the cell lysis was detected in ΔP2345 with the addition of 4 µg/mL of mitomycin C at the OD₆₀₀ of 0.2-0.5 (FIG. 12), while no cell lysis was detected in any other mutants at any conditions with the treatment using mitomycin C or norfloxacin (data not shown). Furthermore, after induction, phage-like particles were only observed in the supernatant of ΔP2345 (FIG. 5g & FIG. 13) and they were likely the HM T phages. However, the phage image was different from what was described before¹⁷. It was more like HM 7 (a head with a long tail), rather than HM 1 (a head with multiple short tails). It is worthwhile to mention that this is the first time that an image of the HM T phage has been reported.

After the deletion of P5, no clostocin O particle was observed in the supernatant of ΔP12345, which confirmed that P5 indeed encoded clostocin O. In addition, as mentioned above, no cell lysis was observed in ΔP12345 with induction (FIG. 5i & FIG. 12), suggesting that ΔP12345 could be a more stable platform to be engineered for enhanced ester production. On the other hand, we showed above that ΔP1234 grew faster and produced more butanol than the control N1-4-C strain. Therefore, we further compared the fermentation performance of ΔP1234 vs ΔP12345 in both serum bottles and bioreactors (FIGS. 5d, 5e, S8, & S9). Results showed that the further deletion of P5 in ΔP12345 did not result in significant difference in cell growth or butanol production when compared to ΔP1234; actually the butanol production in ΔP12345 was slightly lower than in ΔP1234.

### Discussion

We firstly set out to screen the host strains and ester synthesis genes for specific ester production. With the combination of five clostridial strains (C. saccharoperbutylacetonicum N1-4-C, C. pasteurianum ester SD-1, C. beijerinckii 8052, C. tyrobutyricum cat1::adhE1 and cat1::adhE2) and five ester synthesis genes (vaat, saat, atf1, eht1 and lipaseB), we obtained very promising results. Most of the engineered strains could produce EA, BA and BB at the same time (FIG. 2), and some of the strains could also produce small amount of EB. C. saccharoperbutylacetonicum FJ-004 produced 5.5 g/L BA, which was the highest BA production level that has ever been reported¹⁸. C. pasteurianum J-5 produced 0.3 g/L BB, which was also significantly higher than the previously reported level of 0.05 g/L in an engineered C. acetobutylicum strain^{18, 19}. The results confirmed our hypothesis that solventogenic clostridia are outstanding platforms to be engineered for ester production. Because the BA production in FJ-004 was significantly higher than the production levels of other esters, we decided to focus on further improving BA production in C. saccharoperbutylacetonicum through systematic metabolic engineering.

Butanol and acetyl-CoA are the two precursors for BA synthesis. The enhancement of the intracellular pool of these two precursors in the host could help improve BA production. We thus firstly deleted nuoG to save NADH and improve butanol and thus BA production. Our fermentation results showed that FJ-101 with the deletion of nuoG had increased BA production to 7.8 g/L. However, there was still 7.6 g/L butanol remaining at the end of fermentation with FJ-101; it was thus reasonable to speculate that the availability of acetyl-CoA was the bottleneck for further improving BA production. We tried two strategies to improve intracellular acetyl-CoA availability. One was for the enhanced ‘regeneration’ of acetyl-CoA, and the other was for ‘blocking’ the pathway that consumes acetyl-CoA. Comparatively, the former seemed a better strategy. By introducing a heterologous isopropanol synthesis pathway to promote the ‘regeneration’ of intracellular acetyl-CoA, the FJ-301 strain could produce up to 12.9 g/L BA (FIG. 3b).

The dynamic expression of the heterologous pathway to be synchronous with the production of the precursors could be highly beneficial for the production of the target bioproduct. On the other hand, the imbalance of intracellular metabolism and the accumulation of toxic precursors would harm the cells and lead to decreased production of the target product. We hypothesized that the appropriate regulation of the BA synthesis enzyme using the native promoter of the host strain could achieve the similar effect as DSRS. In this work, four native promoters associated with BA precursors formation were selected and evaluated to control the atf1 gene expression (FIG. 3c). Results indicated that the atf1 gene controlled by the P_{adh} promoter showed 10.5% increase in BA production compared with the control FJ-301 strain (FIG. 3d). The P_{adh} promoter is responsible for the ethanol and butanol synthesis. The synchronous expression of alcohol dehydrogenase and ATF1 remarkably increased BA production.

Spatial organization of the enzymes associated with BA synthesis is another strategy that we employed to enhance BA production. The cross-link of the enzymes associated with BA synthesis or anchoring these enzymes onto a synthetic scaffold (PduA*) was not able to improve the BA production; while anchoring the ATF1 enzyme to the cell membrane by adding a MinD C-tag to the C-terminus of the enzyme led to significantly increased BA production. The obtained FJ-308 produced 16.4 g/L BA, which was 20% more than that in FJ-304 (FIG. 4). The attachment of ATF1 to the cell membrane facilitated the excretion of BA from the cells, which could mitigate the intracellular toxicity caused by BA and meanwhile boost the BA synthesis.

During the fermentation, the performance of the strain for BA production was not stable, and remarkable cell lysis was also observed at the end of the fermentation. We speculated that the instability of the strain could be because of the prophages existing in the chromosome of C. saccharoperbutylacetonicum. Based on analysis, we identified four putative prophages P1-P4 and one incomplete prophage genome P5 in the genome of C. saccharoperbutylacetonicum N1-4 (HMT). P5 was demonstrated to be responsible for the synthesis of clostocin O (FIGS. 5F & S5). Ultimately, we obtained two mutant strains ΔP1234 and ΔP12345, both of which grew faster and produced more butanol than the wild type strain (FIGS. 5b-e, S3, S8, & S9). Thus, we further constructed the BA-producing strains FJ-1201 and FJ-1301 respectively based on ΔP1234 and ΔP12345. Fermentation results demonstrated that FJ-1201 could produce 20.3 g/L BA (FIG. 6A & Table S3), which was the highest production level of BA known to us in any microbial biocatalyst host^{1B}. The BA yield in FJ-1201 reached 0.26 g/g, which was also significantly higher than the initial BA-producing strain FJ-004 (0.07 g/g). Thus, the deletion of prophages from C. saccharoperbutylacetonicum could not only increase the cell growth (and stability) but also the production of the desired end products (butanol or BA).

In addition, we also noticed that the BB production in FJ-1201 reached 0.9 g/L, which was significantly higher than that in C. pasteurianum J-5 (0.3 g/L, the highest BB production level based on our initial screening of the strains and enzymes for ester production) (FIG. 6A & Table S3). We further overexpressed saat (instead of atf1) in the strain, and obtained the FJ-1202 strain in which the BB production reached unprecedented 1.3 g/L (FIG. 6D).

Both the BA-producing C. saccharoperbutylacetonicum FJ-1201 and the BB-producing C. saccharoperbutylacetonicum FJ-1202 performed well when biomass hydrolysates was used as the substrate for the fermentation. FJ-1201 could produce 17.8 g/L BA and FJ-1202 could produce 0.9 g/L BB from biomass hydrolysates (with no need to supplement any exogenous nitrogen source). Although these levels were slightly lower than when glucose was used as the substrate for the fermentation with the same strain, the operation eliminated the requirement of yeast extract and tryptone and thus would significantly decrease the cost of the bioprocess for fatty acid ester production.

TABLE S1

Strains and plasmids used in this study

| Strains and plasmids | Relevant genotype and characteristics | Source |
|---|---|---|
| Strains | | |
| C. saccharoperbutylacetonicum N1-4 (HMT) | DSM 14923 (= ATCC 27021), wild-type strain | DSMZ |
| C. tyrobutyricum Δcat1::adhE1 | ATCC 25755, cat1::adhE1 | 4 |
| C. tyrobutyricum Δcat1::adhE2 | ATCC 25755, cat1::adhE2 | 4 |
| C. pasteurianum SD-1 | ATCC 6013 with the deletion of cpaAI gene | Lab stock |
| Clostridium beijerinckii NCIMB 8052 | Wild-type strain | 22 |
| C. saccharoperbutylacetonicum | ACsp 135p (with the endogenous plasmid Csp_135p | 23 |
| N1-4-C | eliminated) | |
| C. saccharoperbutylacetonicum FJ-001 | N1-4-C harboring pMTL82151 | This study |
| C. saccharoperbutylacetonicum FJ-002 | N1-4-C harboring pMTL-P_{cat}-vaat | This study |
| C. saccharoperbutylacetonicum FJ-003 | N1-4-C harboring pMTL-P_{cat}-saat | This study |
| C. saccharoperbutylacetonicum FJ-004 | N1-4-C harboring pMTL-P_{cat}atf1 | This study |
| C. saccharoperbutylacetonicum FJ-005 | N1-4-C harboring pMTL-P_{cat}-eht1 | This study |
| C. saccharoperbutylacetonicum FJ-006 | N1-4-C harboring pMTL-P_{cat}-lipaseB | This study |
| C. saccharoperbutylacetonicum FJ-007 | N1-4-C harboring pMTL-P_{cat}-atf1′ | This study |
| C. saccharoperbutylacetonicum FJ-008 | N1-4-C harboring pMTL-Pₜₕₗ-atf1 | This study |
| C. saccharoperbutylacetonicum FJ-100 | N1-4-C, ΔnuoG | This study |
| C. saccharoperbutylacetonicum FJ-101 | FJ-100 harboring pMTL-P_{cat}-atf1 | This study |
| C. saccharoperbutylacetonicum FJ-200 | FJ-100 with Pₜₕᵢ-_{sadh} integrated at Cspa_c21540 site | This study |
| C. saccharoperbutylacetonicum FJ-201 | FJ-200 harboring pMTL-P_{cat}-atf1 | This study |
| C. saccharoperbutylacetonicum FJ-300 | FJ-100 with Pₜₕₗ-sadh-hydG integrated at Cspa c21540 site | This study |
| C. saccharoperbutylacetonicum FJ-301 | FJ-300 harboring pMTL-P_{cat}-atf1 | This study |
| C. saccharoperbutylacetonicum FJ-302 | FJ-300 harboring pMTL-P_{pfl}-atf1 | This study |
| C. saccharoperbutylacetonicum FJ-303 | FJ-300 harboring pMTL-P_{ald}-atf1 | This study |
| C. saccharoperbutylacetonicum FJ-304 | FJ-300 harboring pMTL-P_{adh}-atf1 | This study |
| C. saccharoperbutylacetonicum FJ-305 | FJ-300 harboring pMTL-P_{bdh}-atf1 | This study |
| C. saccharoperbutylacetonicum FJ-306 | FJ-300 harboring pMTL-P_{adh}-A-atf1 | This study |
| C. saccharoperbutylacetonicum FJ-307 | FJ-300 harboring pMTL-P_{adh}-A-atf1-MinD | This study |
| C. saccharoperbutylacetonicum FJ-308 | FJ-300 harboring pMTL-P_{adh}-atf1-MinD | This study |
| C. saccharoperbutylacetonicum FJ-309 | FJ-300 harboring pMTL-P_{adh}-A-atf1-B-nifJ | This study |
| C. saccharoperbutylacetonicum FJ-310 | FJ-300 harboring pMTL-P_{adh}-A-atf1-B-nifJ-B-bdhA | This study |
| C. saccharoperbutylacetonicum FJ-311 | FJ-300 harboring pTJI1-P_{cat}-B-pduA* and pMTL-P_{adh}- A-atfl | This study |
| C. saccharoperbutylacetonicum FJ-312 | FJ-300 harboring pTJI1-P_{cat}-B-pduA*-A-nifJ and pMTL-P_{adh}-A-atf1 | This study |
| C. saccharoperbutylacetonicum FJ-313 | FJ-300 harboring pTJ1-P_{cat}-B-pduA*-A-nifJ-A-bdhA and pMTL-P_{adh}-A-atf1 | This study |
| C. saccharoperbutylacetonicum FJ-400 | FJ-300 derivative, ACspa_c20890 | This study |
| C. saccharoperbutylacetonicum FJ-401 | FJ-400 harboring pMTL-P_{cat}-atf1 | This study |
| C. saccharoperbutylacetonicum FJ-500 | FJ-300 derivative, ACspa_c50320 | This study |
| C. saccharoperbutylacetonicum FJ-501 | FJ-500 harboring pMTL-P_{cat}-atf1 | This study |
| C. saccharoperbutylacetonicum FJ-1100 | ΔP1234 derivative with the deletion of nuoG | This study |
| C. saccharoperbutylacetonicum FJ-1200 | FJ-1100 derivative with the insertion of Pₜₕₗ-sadh- hydG | This study |
| C. saccharoperbutylacetonicum FJ-1201 | FJ-1200 harboring pMTL-P_{adh}-atf1-MinD | This study |
| C. saccharoperbutylacetonicum FJ-1300 | ΔP12345 derivative with the deletion of nuoG and insertion of Pₜₕₗ-Sadh-hydG | This study |
| C. saccharoperbutylacetonicum FJ-1301 | FJ-1300 harboring pMTL-P_{adh}-atf1-MinD | This study |
| C. saccharoperbutylacetonicum FJ-1202 | FJ-1200 harboring pMTL-P_{cat}-saat | This study |
| C. saccharoperbutylacetonicum FJ-1203 | FJ-1200 harboring pMTL-P_{cat}-eht1 | This study |
| C. saccharoperbutylacetonicum FJ-1204 | FJ-1200 harboring pMTL-Pcat-saat-MinD | This study |
| C. saccharoperbutylacetonicum YM016 | FJ-1200 derivative with the deletion of rex | This study |
| C. saccharoperbutylacetonicum YM016PB | YM016 harboring pMTL-P_{cat}-saat | This study |
| C. saccharoperbutylacetonicum YM028 | YM-016 derivative, Δldh1l:: P_{adh}-atf1-MinD, ΔlacZ1:: P_{adh}-atf1-MinD, ΔlacZ2::P_{adh}-atf1-MinD, ΔctfA1-ctfB1 | This study |
| C. saccharoperbutylacetonicum YM028P | YM028 harboring pMTL-P_{adh}-atf1-MinD | This study |
| C. saccharoperbutylacetonicum ΔP1 | N1-4-C strain with the deletion of prophage HM T (P1) | This study |
| C. saccharoperbutylacetonicum ΔP2 | N1-4-C strain with the deletion of putative phage P2 | This study |
| C. saccharoperbutylacetonicum ΔP3 | N1-4-C strain with the deletion of putative phage P3 | This study |
| C. saccharoperbutylacetonicum ΔP4 | N1-4-C strain with the deletion of putative phage P4 | This study |
| C. saccharoperbutylacetonicum ΔNP1 | N1-4-C strain with the deletion of Cspa_c09880 (NP1) gene | This study |
| C. saccharoperbutylacetonicum ΔNP2 | N1-4-C strain with the deletion of Cspa_c26510 (NP2) gene | This study |
| C. saccharoperbutylacetonicum ΔNP3 | N1-4-C strain with the deletion of Cspa_c36800 (NP3) gene | This study |
| C. saccharoperbutylacetonicum ΔNP4 | N1-4-C strain with the deletion of Cspa_c57380 (NP4) gene | This study |
| C. saccharoperbutylacetonicum ΔNP1234 | N1-4-C strain with the deletion of Cspa_c09880, Cspa c26510, Cspa c36800 and Cspa c57380 | This study |
| C. saccharoperbutylacetonicum ΔP123 | N1-4-C strain with the deletion of putative prophages P1, P2 and P3 | This study |
| C. saccharoperbutylacetonicum ΔP124 | N1-4-C strain with the deletion of putative prophages P1, P2 and P4 | This study |
| C. saccharoperbutylacetonicum ΔP134 | N1-4-C strain with the deletion of putative prophages P1, P3 and P4 | This study |
| C. saccharoperbutylacetonicum ΔP234 | N1-4-C strain with the deletion of putative prophages P2, P3 and P4 | This study |
| C. saccharoperbutylacetonicum ΔP1234 | N1-4-C strain with the deletion of putative prophages P1, P2, P3 and P4 | This study |
| C. saccharoperbutylacetonicum ΔP2345 | N1-4-C strain with the deletion of putative prophages P2, P3, P4 and P5 | This study |
| C. saccharoperbutylacetonicum ΔP1345 | N1-4-C strain with the deletion of putative prophages P1, P3, P4 and P5 | This study |
| C. saccharoperbutylacetonicum ΔP1245 | N1-4-C strain with the deletion of putative prophages P1, P2, P4 and P5 | This study |
| C. saccharoperbutylacetonicum ΔP1235 | N1-4-C strain with the deletion of putative prophages P1, P2, P3 and P5 | This study |
| C. saccharoperbutylacetonicum AP12345 | N1-4-C strain with the deletion of putative prophages P1, P2, P3, P4 and P5 | This study |
| C. pasteurianum J-1 | SD-1 harboring pMTL82151 | This study |
| C. pasteurianum J-2 | SD-1 harboring pMTL-P_{cat}-vaat | This study |
| C. pasteurianum J-3 | SD-1 harboring pMTL-P_{cat}-saat | This study |
| C. pasteurianum J-4 | SD-1 harboring pMTL-P_{cat}-atf1 | This study |
| C. pasteurianum J-5 | SD-1 harboring pMTL-Pcat-eht1] | This study |
| C. pasteurianum J-6 | SD-1 harboring pMTL-P_{cat}-lipaseB | This study |
| Clostridium beijerinckii F-1 | NCIMB 8052 harboring pTJ1 | This study |
| Clostridium beijerinckii F-2 | NCIMB 8052 harboring pTJ1-P_{cat}-vaat | This study |
| Clostridium beijerinckii F-3 | NCIMB 8052 harboring pTJ1-P_{cat}-saat | This study |
| Clostridium beijerinckii F-4 | NCIMB 8052 harboring pTJ1-P_{cat}-atf1 | This study |
| Clostridium beijerinckii F-5 | NCIMB 8052 harboring pTJ1-P_{cat}-eht1 | This study |
| Clostridium beijerinckii F-6 | NCIMB 8052 harboring pTJ1-P_{cat}-lipaseB | This study |
| C. tyrobutyricum JZ-1 | Δcat1::adhE1 harboring pMTL82152 | This study |
| C. tyrobutyricum JZ-2 | Δcat1::adhE1 harboring pMTL-P_{cat}-vaat | This study |
| C. tyrobutyricum JZ-3 | Δcat1::adhE1 harboring pMTL-P_{cat}-saat | This study |
| C. tyrobutyricum JZ-4 | Δcat1::adhE1 harboring pMTL-P_{cat}-atf1 | This study |
| C. tyrobutyricum JZ-5 | Δcat1::adhE1 harboring pMTL-P-_{cat}-eht1 | This study |
| C. tyrobutyricum JZ-6 | Δcat1::adhE1 harboring pMTL-P_{cat}-lipaseB | This study |
| C. tyrobutyricum JZ-7 | Δcat1::adhE2 harboring pMTL82152 | This study |
| C. tyrobutyricum JZ-8 | Δcat1::adhE2 harboring pMTL-P_{cat}-vaat | This study |
| C. tyrobutyricum JZ-9 | Δcat1::adhE2 harboring pMTL-P_{cat}-saat | This study |
| C. tyrobutyricum JZ-10 | Δcat1::adhE2 harboring pMTL-P_{cat-}atf1 | This stud |
| C. tyrobutyricum JZ-11 | Δcat1::adhE2 harboring pMTL-P_{cat}-eht1 | This study |
| C. tyrobutyricum JZ-12 | Δcat1::adhE2 harboring pMTL-P_{cat}-lipaseB | This study |
| E. coli CA434 | hsd20(rB-, mB-), recA13, rpsL20, leu, proA2, with IncPb conjugative plasmid R702 | 24 |
| E. coli DH5α | F⁻, φp80dlacZΔM1, Δ(lacZYA-argF)U169, deoR, recA1, endA1, hsdr17(rₖ⁻, mₖ⁺), phoA, supE44, λ⁻ thi-1, gyrA96, relA1 | NEB |
| Plasmids | | |
| pYW34 | CAK ori, Amp^{r}, Erm^{r}, Plac::Cas9, gRNA | 3 |
| pMTL82151 | pBP1 ori, catP, Co1E1, tra | 25 |
| pYW 34-ΔnuoG | Derivative of pYW34 for nuoG deletion | This study |
| pYW34-sadh | Derivative of pYW34 for Pₜₕₗ₋sadh integration | 26 |
| pYW 34-sadh-hvdG | Derivative of pYW34 for Pₜₕᵢ-_{sadh}-h_{vdG} integration | 26 |
| pYW34-ΔCspa c06180 | Derivative of pYW34 for Cspa c06180 deletion | This study |
| pYW34-ΔCspa c17310 | Derivative of pYW34 for Cspa c17310 deletion | This study |
| pYW34-ΔCspa c20520 | Derivative of pYW34 for Cspa c20520 deletion | This study |
| pYW34-ΔCspa c20890 | Derivative of pYW34 for Cspa c20890 deletion | This study |
| pYW34-ΔCspa_c50320 ^{f} | Derivative of pYW34 for Cspa_c50320 deletion | This study |
| pYW34-API | Derivative of pYW34 for P I phage deletion | This study |
| pYW34-AP2 | Derivative of pYW34 for P2 phage deletion | This study |
| pYW34-ΔP3 | Derivative of pYW34 for P3 phage deletion | This study |
| pYW34-AP4 | Derivative of pYW34 for P4 phage deletion | This study |
| pYW34-AP5 | Derivative of pYW34 for P5 phage deletion | This study |
| pYW 34-ΔNP 1 | Derivative of pYW34 for Cspa_c09880 deletion | This study |
| pYW34-ΔNP2 | Derivative of pYW34 for Cspa_c26510 deletion | This study |
| pYW-34-ΔNP3 | Derivative of pYW34 for Cspa_c36800 deletion | This study |
| pYW34-ΔNP4 | Derivative of pYW34 for Cspa_c57380 deletion | This study |
| pMTL82151-P_{cat} | =pJZ98-Pcat1, pMTL82151 derivative, containing P_{cat} for gene overexpression purpose | 4 |
| pMTL82151-Pₜₕₗ | pMTL82151 derivative, containing Pₜₕₗ for gene overexpression purpose | Lab stock |
| pMTL8215 I-P_{pfl} | pMTL82151 derivative, containing P_{pfl} for gene overexpression purpose | This study |
| pMTL82151-Pbld | pMTL82151 derivative, containing P_{bld} for gene overexpression purpose | This study |
| pMTL82151-P_{adh} | pMTL82151 derivative, containing P_{adh} for gene overexpression purpose | This study |
| pMTL82151-P_{bdh} | pMTL82151 derivative, containing P_{bdh} for gene overexpression purpose | This study |
| pMTL-P_{cat}-vaat | pMTL82151-P_{cat} derivative, with vaat inserted downstream of P_{cat} | This study |
| pMTL-P_{cat}-saat | pMTL82151-P_{cat} derivative, with saat inserted downstream of P_{cat} | This study |
| pMTL-Peat-atf1 | pMTL82151-P_{cat} derivative, with atf1 inserted downstream of P_{cat} | This study |
| pMTL-Pcat-ehtJ | pMTL82151-P_{cat} derivative, with eht1 inserted downstream of P_{cat} | This study |
| pMTL-P_{cat}-lipaseB | pMTL82151-P_{cat} derivative, with lipaseB inserted | This study |
| | downstream of P_{cat} | |
| pMTL-Peat-atf1′ | pMTL82151-P_{cat} derivative, with atf1′ inserted downstream of P_{cat} | This study |
| pMTL-Pₜₕₗ-atf1 | pMTL82151-Pₜₕₗ derivative, with atf1 inserted downstream of Pₜₕₗ | This study |
| pMTL-P_{pfl}-atf] | pMTL82151-P_{pfl} derivative, with atf1 inserted downstream of P_{pfl} | This study |
| pMTL-P_{bld}-atf1 | pMTL82151-P_{bld} derivative, with atf1 inserted downstream of P_{bld} | This study |
| _{P}MTL-P_{adh}-atf1 | pMTL82151-P_{adh} derivative, with atf1 inserted downstream of P_{adh} | This study |
| pMTL-Pbdh-atf1 | pMTL82151-P_{bdh} derivative, with atf1 inserted downstream of P_{bdh} | This study |
| pMTL-P_{adh}-CC-Di-A | pMTL82151-P_{adh} derivative, CC-Di-A inserted downstream of P_{adh} | This study |
| pMTL-P_{adh}-atf1-MinD | pMTL82151-P_{adh} derivative, with atf1-MinD inserted downstream of P_{adh} | This study |
| pMTL-Peat-saat-MinD | pMTL82151-P_{cat} derivative, with saat-MinD inserted downstream of P_{cat} | This study |
| pMTL-P_{adh}-A-atf1 | pMTL82151-P_{adh} derivative, with CC-Di-A-atf1 fragment inserted downstream of P_{adh} | This study |
| pMTL-P_{adh}-A-atf1-MinD | pMTL82151-P_{adh} derivative, with CC-Di-A-atf1-MinD fragment inserted downstream of P_{adh} | This study |
| pMTL-P_{adh}-A-atf1-B-nzfJ | pMTL-P_{adh}-A-atf1 derivative, with CC-Di-B-nifJ fragment inserted downstream of the atf1 gene | This study |
| pMTL-P_{adh}-A-atf1-B-nifJ-B- bdhA | pMTL-P_{adh}-A-atf1-B-nifJ derivative, with CC-Di-B-bdhA fragment inserted downstream of the nifJ gene | This study |
| pMTL-P_{adh}-A-atfl-MinD-B-nifJ | pMTL-P_{adh}-A-atf1-MinD derivative, with CC-Di-B-nifJ fragment inserted downstream of the atf1-MinD gene | This study |
| pMTL-P_{adh}-A-αtf1-MinD-B-nifJ- B-bdhA | pMTL-P_{adh}-A-atf1-MinD-B-nifJ derivative, with CC-Di-B-bdh4 fragment inserted downstream of the atf1-MinD gene | This study |
| pTJ1-Peat-B -pduaA* | pTJ1-P_{cat} derivative with the insertion of CC-Di-B-pduA* | This study |
| pTJ1-Pcat-B-pduA*-A-nifJ | pTJ1-P_{cat-}B-pduA* derivative with the insertion of CC-Di-A-nifJ | This study |
| pTJ1-Peat-B-pduA*-A-nifJ-A-bdhA | pTJ1-P_{cat-}B-pduA*-A-nifJ derivative with the insertion of CC-Di-A-bdhA | This study |
| pTJ1 | pYL102E derivative, Amp^{r}, Erm^{r} | 22 |
| pTJ1-Peat | pTJ1 derivative, with the insertion of P_{cat} promoter | This study |
| pTJ1-Peat-vaat | pTJ1 derivative, with the insertion of P_{cat}-vaat | This study |
| pTJ1-P_{cat}-saat | pTJ1 derivative, with the insertion of P_{cat-}saat | This study |
| pTJ1-P_{cat}-atf1 | pTJ1 derivative, with the insertion of P_{cat}-atf1 | This study |
| pTJ1-P_{cat}-eht1 | pTJ1 derivative, with the insertion of P_{cat}-eht1 | This study |
| pTJ1-P_{cat}-lipaseB | pTJ1 derivative, with the insertion of P_{cat}-lipaseB | This study |
| pDL004 | pETite* carrying atf1 from S. cerevisiae | 27 |
| pDL001 | pETite* carrying saat from Fragaria ananassa | 28 |
| pDL006 | pETite* carrying vaat from F. vesca | 27 |
| pYW34-Δrex | Derivative of pYW34 for rex deletion | |
| pYW34-Δldh1::P_{adh}-atf1-MinD | Derivative of pYW34 for ldh1 deletion and Padh-atfl-MinD cassette integration | |
| pYW34-ΔlacZ1::P_{adh}-atf1-MinD | Derivative of pYW34 for lacZ1 deletion and Padh-atfl-MinD cassette integration | |
| pYW34-ΔlαcZ2::P_{adh}-atf1-MinD | Derivative of pYW34 for lacZ2 deletion and Padh-atfl-MinD cassette integration | |
| pYW 4-ΔctfA1-ctfB1 | Derivative of pYW34 for ctfA1-ctfB1 deletion | |

TABLE S2

Primers Used In Working Example

| Primers | Sequence (5′ to 3′) | SEQ ID NO. | Purpose |
|---|---|---|---|
| nuoG-UF | TGATATGACTAATAATTAGCGGCCGCTAGTATTTTCA GGAATTTCTCCAAT | 8 | pYW34-ΔnuoG |
| nuoG-UR | TTGAAAGACATTAAGAGGGGCAACGATTAACTATGA TAAATG | 9 | |
| nuoG-DF | TTAATCGTTGCCCCTCTTAATGTCTTTCAAACTAAAT ACC | 10 | |
| nuoG-DR | ACTAGTAACCATCACACTGTAAGATAACCTTTGAATT GTTACGC | 11 | |
| nuoG-U | ATTGCTTGAATTTCGTCTTCAAAAC | 12 | |
| nuoG-D | TAGATAACATGATATCCAAGGTCGC | 13 | |
| nuoG-20nt | GCTCAGTCCTAGGTATAATGCTAGCTTTACTTCATCT TTTGAAGCGTTTTAGAGCTAGAAATAGCAAG | 14 | |
| YW1139 | GTCATAAACTTGCTCAACTGATATGATCTTCCATAAC TTAAC | 15 | gRNA amplificati on |
| YW1142 | ATATAGGTAATCGCTTTCATAAGGGCCCGATCGGTCT TGCCTTGCTCGTCG | 16 | |
| Cspa_c06180-UF | TGATATGACTAATAATTAGCGGCCGCCCCCATTAGA TACTAAAGAAACTCA | 17 | pYW34-ΔCspa_c06 180 |
| Cspa_c06180-UR | CTAAACTCTTTCAACAACTACGTCTCTCATGTTTGAC CTC | 18 | |
| Cspa_c06180-DF | ATGAGAGACGTAGTTGTTGAAAGAGTTTAGTATACA AGTT | 19 | |
| Cspa_c06180-DR | ACTAGTAACCATCACACTGGGTTTGTAAAAGCAGTTT ATTCATC | 20 | |
| Cspa_c06180-U | GTTTTTGAGAGATTAGGTGGAAAGG | 21 | |
| Cspa_c06180-D | TCCCTACTGCATAGATTCCTGCATT | 22 | |
| Cspa_c06180-20nt | GCTCAGTCCTAGGTATAATGCTAGCTCAAAAGTAAA TGTTAATGGGTTTTAGAGCTAGAAATAGCAAG | 23 | |
| Cspa_c17310-UF | TGATATGACTAATAATTAGCGGCCGCCAGACAGTGG CCTTATCAATAGGAG | 24 | pYW34-ΔCspa_c17 310 |
| Cspa_c17310-UR | TTATAATCTTTCTACAACTACTTCTTTCATTTATGCTC CT | 25 | |
| Cspa_c17310-DF | ATGAAAGAAGTAGTTGTAGAAAGATTATAATAATAG ATAA | 26 | |
| Cspa_c17310-DR | ACTAGTAACCATCACACTGCCTCTTTTTTCTTTTTCAT TGCTTC | 27 | |
| Cspa_c17310-U | TCAGGGACAGCAGTTCATGTAGTAA | 28 | |
| Cspa_c17310-D | TTATGGTAAAGCCTAATACAAACCC | 29 | |
| Cspa_c17310-20nt | GCTCAGTCCTAGGTATAATGCTAGCAAGAAAGTTAA | 30 | |
| | TGTAAGTGGGTTTTAGAGCTAGAAATAGCAAG | | |
| Cspa_c20520-UF | TGATATGACTAATAATTAGCGGCCGCAAGACATTAA AGATTCTGCAACAGC | 31 | pYW34-ΔCspa_c20 520 |
| Cspa_c20520-UR | TTATTCTCTTTCAACTACTACATCTTTCATTTTTTATT CCTC | 32 | |
| Cspa_c20520-DF | ATGAAAGATGTAGTAGTTGAAAGAGAATAATTGAAT TTAA | 33 | |
| Cspa_c20520-DR | ACTAGTAACCATCACACTGATGCAAGATACACAAGC TATAGGAT | 34 | |
| Cspa_c20520-U | TTTTATGTCATAGCAATTGAGGGAA | 35 | |
| Cspa_c20520-D | TTGTTCTTTATATTTTTCATTGGGC | 36 | |
| Cspa_c20520-20nt | GCTCAGTCCTAGGTATAATGCTAGCCTTGCAACCTTG TGTATTGGGTTTTAGAGCTAGAAATAGCAAG | 37 | |
| Cspa_c20890-UF | TGATATGACTAATAATTAGCGGCCGCAATAACTGAT GTAGCCAGTGCAACT | 38 | pYW34-ΔCspa_c20 890 |
| Cspa_c20890-UR | TCAATTGCATCTTTCTACTACTTCTTTCATTTTTAACT CC | 39 | |
| Cspa_c20890-DF | ATGAAAGAAGTAGTAGAAAGATGCAATTGATAGAG GAATG | 40 | |
| Cspa_c20890-DR | ACTAGTAACCATCACACTGAATTTTCTTACCGCTACA AAGATCA | 41 | |
| Cspa_c20890-U | AATATTTGCTGAACTTGATGACGTA | 42 | |
| Cspa_c20890-D | ACCATTTAATACATAATGGTCACCT | 43 | |
| Cspa_c20890-20nt | GCTCAGTCCTAGGTATAATGCTAGCTGTTAAATGATG CAAGATGGGTTTTAGAGCTAGAAATAGCAAG | 44 | |
| Cspa_c50320-UF | TGATATGACTAATAATTAGCGGCCGCTGACAATAAT GTGTTTAAATTTCCT | 45 | pYW34-ΔCspa_c50 320 |
| Cspa_c50320-UR | GTGGAAAGTGTATATATTAAAAGGAGCTAAGGATGA ACTAT | 46 | |
| Cspa_c50320-DF | TTAGCTCCTTTTAATATATACACTTTCCACTTATAAA ACT | 47 | |
| Cspa_c50320-DR | ACTAGTAACCATCACACTGCTATTTTAGGATATGTTG ATAATGC | 48 | |
| Cspa_c50320-U | TTTCATTGATTTTATCCATCTTCTT | 49 | |
| Cspa_c50320-D | TTAATCCTATTTATTCAGATGAAGA | 50 | |
| Cspa_c50320-20nt | GCTCAGTCCTAGGTATAATGCTAGCAACTGTGCTACT TGAAAATAGTTTTAGAGCTAGAAATAGCAAG | 51 | |
| P1-UF | TGATATGACTAATAATTAGCATCAACCAGGGAATTT ATAAGAAGC | 52 | pYW34-ΔP1 |
| P1-UR | AGCTGCTAATTTAGTATCAGTCTTCATTTAATGGAAT TTACATTAA | 53 | |
| P1-DF | TAAATGAAGACTGATACTAAATTAGCAGCTGAAAAT GAAAGT | 54 | |
| P1-DR | ACTAGTAACCATCACACTGGCCCAAAATTCCTTTAAC TTGCTCGTA | 55 | |
| P1-U | TATTAGTGCAAAATGGCTTTATGAC | 56 | |
| P1-D | GCATCTGAATCCATTATTTTAGTTT | 57 | |
| P1-20nt | GCTCAGTCCTAGGTATAATGCTAGCGATCATATATTG CTAAACATGTTTTAGAGCTAGAAATAGCAAG | 58 | |
| P2-UF | TGATATGACTAATAATTAGCGAAGAGAAAGAAAGCA AATATTACC | 59 | pYW34-ΔP2 |
| P2-UR | ATAGCTCCTATCTTTGTGTTAAAAATGTGTTAATAAT AAA | 60 | |
| P2-DF | TTAACACATTTTTAACACAAAGATAGGAGCTATATAT GAAA | 61 | |
| P2-DR | ACTAGTAACCATCACACTGGCTTTAACTTCGATCCTC TTAGCACCT | 62 | |
| P2-U | CCAAGTGAAAAAATTGAATTTGATA | 63 | |
| P2-D | AATTTTTATTAAGACCCTGCCGCTA | 64 | |
| P2-20nt | GCTCAGTCCTAGGTATAATGCTAGCGGAGCTGCAAC AACTATAAAGTTTTAGAGCTAGAAATAGCAAG | 65 | |
| P3-UF | TGATATGACTAATAATTAGC GCTTAATATTGTCTATTGATACCGA | 66 | pYW34-ΔP3 |
| P3-UR | GAATAAAAGATGATTAGATATGCTCGAATTTGATTA TTATAA | 67 | |
| P3-DF | AATTCGAGCATATCTAATCATCTTTTATTCTATACAC ATTA | 68 | |
| P3-DR | ACTAGTAACCATCACACTGGCTTATAAATCTTTATGG GGCAAGAGG | 69 | |
| P3-U | TTTTCCAAATAATTTGCTTAATCCT | 70 | |
| P3-D | CAAGACAACTAATTATCGAAAAACA | 71 | |
| P3-20nt | GCTCAGTCCTAGGTATAATGCTAGCTCACCTCCATAT TTTCTTCTGTTTTAGAGCTAGAAATAGCAAG | 72 | |
| P4-UF | TGATATGACTAATAATTAGCCAGAGCTTTGTGTAATT GATGTAAC | 73 | pYW34-ΔP4 |
| P4-UR | TATATTATCAAGCAAATAACCCGTAAGTAACAATTA AGCT | 74 | |
| P4-DF | TTACTTACGGGTTATTTGCTTGATAATATATTTAAAT ATA | 75 | |
| P4-DR | ACTAGTAACCATCACACTGGCCTAATGAAGCCAATA TGGAGAAACT | 76 | |
| P4-U | ATGCAACATACCGGAAAAGGTAAAC | 77 | |
| P4-D | AATAGATAGGTTGCAAAAGGAAATG | 78 | |
| P4-20nt | GCTCAGTCCTAGGTATAATGCTAGCTCTCTTAAATCA CTCATTATGTTTTAGAGCTAGAAATAGCAAG | 79 | |
| P5-UF | TGATATGACTAATAATTAGCATTTGTATGAAGGTAGC GGTTGAGG | 80 | pYW34-ΔP5 |
| P5-UR | TAAGATAGTTTAGTTTCCTTTACAAATTAATGTATTT TTTAA | 81 | |
| P5-DF | TTAATTTGTAAAGGAAACTAAACTATCTTAGAAGAC AGAC | 82 | |
| P5-DR | ACTAGTAACCATCACACTGGCCTTAGGAATGATATT CGAAAAAGCA | 83 | |
| P5-U | TCTTTCCTTATAAAGAGTATCGTTTCTAA | 84 | |
| P5-D | CAATTAAAGTATGAACAAAAGCATGG | 85 | |
| P5-20nt | GCTCAGTCCTAGGTATAATGCTAGCTTCAAAATTACA AGCTTCATGTTTTAGAGCTAGAAATAGCAAG | 86 | |
| Cspa_c09880-UF | TGATATGACTAATAATTAGCAATTTCAAGCATAATG GTTATTTCA | 87 | pYW34-ΔNP1 |
| Cspa_c09880-UR | ATGAAAGCAGCTATTAAAGGCGCTCTTTAATGTAGC GCCC | 88 | |
| Cspa_c09880-DF | TTAAAGAGCGCCTTTAATAGCTGCTTTCATAGTATAC CTCC | 89 | |
| Cspa_c09880-DR | ACTAGTAACCATCACACTGGCTCTTAATCAGTCTTCA TTTAATGGA | 90 | |
| Cspa_c09880-U | GGAGATGCTTTTTATGATAAAGAAA | 91 | |
| Cspa_c09880-D | ATATCATTAATACTGATAATGGCGG | 92 | |
| Cspa_c09880-20nt | GCTCAGTCCTAGGTATAATGCTAGCCATTAACCAGTC TTGGTGCAGTTTTAGAGCTAGAAATAGCAAG | 93 | |
| Cspa_c26510-UF | TGATATGACTAATAATTAGCGAAAGCCTCAGGATGG AAAGATAGT | 94 | pYW34-ΔNP2 |
| Cspa_c26510-UR | ATGGCAAATAAAACTTTAAAAATGTGTTAATAATAA AAATAGC | 95 | |
| Cspa_c26510-DF | TTAACACATTTTTAAAGTTTTATTTGCCATAAAATCA TCC | 96 | |
| Cspa_c26510-DR | ACTAGTAACCATCACACTGGCAGAAAACAGAAAGG ATTAACTCAAC | 97 | |
| Cspa_c26510-U | GCTGTGCTGGTATCTAAAATAAAGG | 98 | |
| Cspa_c26510-D | AGGTGAAATTATTGTGTTAGGTGA | 99 | |
| Cspa_c26510-20nt | GCTCAGTCCTAGGTATAATGCTAGCGGAATAGTTACT TTTAAGCAGTTTTAGAGCTAGAAATAGCAAG | 100 | |
| Cspa_c36800-UF | TGATATGACTAATAATTAGCAGCTGACAAATTAAAA GTTAGTGAA | 101 | pYW34-ΔNP3 |
| Cspa_c36800-UR | CTAATTTAGCAACGCAGTCTTTACAGCCATAAGTAA AACC | 102 | |
| Cspa_c36800-DF | ATGGCTGTAAAGACTGCGTTGCTAAATTAGAAATAT AAAC | 103 | |
| Cspa_c36800-DR | ACTAGTAACCATCACACTGGCGGAATGTTTATACCA GTCCTATTCA | 104 | |
| Cspa_c36800-U | AGAAAAATACAGAAATGATCTTCAA | 105 | |
| Cspa_c36800-D | GGTACTTAATTTCTCTTTGGTTTC | 106 | |
| Cspa_c36800-20nt | GCTCAGTCCTAGGTATAATGCTAGCTGATATAGATTA TGATAATAGTTTTAGAGCTAGAAATAGCAAG | 107 | |
| Cspa_c57380-UF | TGATATGACTAATAATTAGCAATTTCTGAACATTTTA AAGGAAGA | 108 | pYW34-ΔNP4 |
| Cspa_c57380-UR | TTTAAATATATTATCCTTATAACTTGCCATAAATACC ACC | 109 | |
| Cspa_c57380-DF | ATGGCAAGTTATAAGGATAATATATTTAAATATAAA CCTC | 110 | |
| Cspa_c57380-DR | ACTAGTAACCATCACACTGGCCTAAGGAAGAAAACA AAATAGCCAA | 111 | |
| Cspa_c57380-U | TACATAAATTCTTTCCAGAAGGGAT | 112 | |
| Cspa_c57380-D | GCCCAATTAAAAAGTGGAGTAGAAG | 113 | |
| Cspa_c57380-20nt | GCTCAGTCCTAGGTATAATGCTAGCATATTTAAATGT TACCCACCGTTTTAGAGCTAGAAATAGCAAG | 114 | |
| pMTL-KZR | AGATCTCCATGGACGCGTGACGTCG | 115 | |
| P_{bld}-F | TCCATATGACCATGATTACGTTAAATAAATTTCCTTA AAAAACAATAT | 116 | pMTL8215 1-P_{bld} |
| P_{bld}-R | ATCCCCGGGTACCGAGCTCGAATTCGACTAATCCTCC TTATGATTTAAAA | 117 | |
| P_{bdh}-F | TCCATATGACCATGATTACGGTTAAGCCACTACCAG GATCAATCA | 118 | pMTL8215 1-P_{bdh} |
| P_{bdh}-R | ATCCCCGGGTACCGAGCTCGAATTCTTCTTTTCCTCC TCTTACACACGCA | 119 | |
| P_{adh}-F | TCCATATGACCATGATTACGACAGGAGATAGCAAAT ACAGAGCTA | 120 | pMTL8215 1-P_{adh} |
| P_{adh}-R | ATCCCCGGGTACCGAGCTCGAATTCAATTTTAATAAC CCTCCTCAAAATA | 121 | |
| P_{pfl}-F | TCCATATGACCATGATTACGTTAGATCCTAAAAAATA TGGCGTTA | 122 | pMTL8215 1-P_{pfl} |
| P_{pfl}-R | ATCCCCGGGTACCGAGCTCGAATTCAAATTAACCAC CTCCCAAATTGAAA | 123 | |
| vaat-F | ATATAATTTATGAAAGGGTGGTTTTTATGGAGAAAA TTGAGGTCAGT | 124 | pMTL-P_{cat}- vaat |
| vaat-R | CGACTCTAGAGGATCCCCGGGTACCGAATTCTCAAT ATCTTGAAATTAGCGTC | 125 | |
| saat-F | ATATAATTTATGAAAGGGTGGTTTTTATGGAGAAAA TTGAGGTCAGT | 126 | pMTL-P_{cat}- saat |
| saat-R | CGACTCTAGAGGATCCCCGGGTACCGAATTCTTAAA TTAAGGTCTTTGGAGATGC | 127 | |
| atf1-F | ATATAATTTATGAAAGGGTGGTTTTTATGAATGAAAT CGATGAGAAAAATCA | 128 | pMTL-P_{cat}- atf1 |
| atf1-R | CGACTCTAGAGGATCCCCGGGTACCGAATTCCTAAG GGCCTAAAAGGAGAGCTTTGT | 129 | |
| eht1-F | ATATAATTTATGAAAGGGTGGTTTTTATGTCAGAAGT ATCTAAGTGG | 130 | pMTL-P_{cat}- eht1 |
| eht1-R | CGACTCTAGAGGATCCCCGGGTACCGAATTCTTATA CAACCAACTCGTCAAAC | 131 | |
| lipaseB-F | ATATAATTTATGAAAGGGTGGTTTTTATGAGAAAAG TATTTTTAAGATC | 132 | pMTL-P_{cat}- lipaseB |
| lipaseB-R | CGACTCTAGAGGATCCCCGGGTACCGAATTCCTAAG GAGTAACAATTCCTGAG | 133 | |
| atf1′-F | ATATAATTTATGAAAGGGTGGTTTTTATGAACGAGAT CGACGAGAAAAACC | 134 | pMTL-P_{cat}- atf1 |
| atf1′-R | CGACTCTAGAGGATCCCCGGGTACCGAATTCTTACG GGCCTAACAGCAGCGC | 135 | |
| atf1-thl-F | ATATAATTTATGAAAGGGTGGTTTTTATGAATGAAAT CGATGAGAAAAATCA | 136 | pMTL-Pₜₕₗ- atf1 |
| atf1-thl-R | CGACTCTAGAGGATCCCCGGGTACCGAATTCCTAAG GGCCTAAAAGGAGAGCTTTGT | 137 | |
| atf1-bld-F | AATCATAAGGAGGATTAGTCATGAATGAAATCGATG AGAAAAATCA | 138 | pMTL8215 1-P_{bld}-atf1 |
| atf1-bdh-F | GTGTAAGAGGAGGAAAAGAAATGAATGAAATCGAT GAGAAAAATCA | 139 | pMTL8215 1-P_{bdh}-atf1 |
| atf1-adh-F | TGAGGAGGGTTATTAAAATTATGAATGAAATCGATG AGAAAAATCA | 140 | pMTL8215 1-P_{adh}-atf1 |
| atf1-pfl-F | ATTTGGGAGGTGGTTAATTTATGAATGAAATCGATG AGAAAAATCA | 141 | pMTL8215 1-P_{pfl}-atf1 |
| atf1-promoter-R | ATCCCCGGGTACCGAGCTCGAATTCCTAAGGGCCTA | 142 | |
| | AAAGGAGAGCTTTGT | | |
| atf1-MinD-R1 | AGCCATCATCCCTTTGTTTTGTTCCTCCAAGACTTGC AACGGCGATCCCGAACCACTAGTAGGGCCTAAAAGG AGAGCTT | 143 | Adding MinD-tag to atf13′ end |
| atf1-MinD-R2 | GATCCCCGGGTACCGAGCTCTTAGGAACGTACACCG AAAAATGATTTGATTTT | 144 | |
| saat-MinD-R1 | GCCATCATCCCTTTGTTTTGTTCCTCCAAGACTTGCA ACGGCGATCCCGAACCACTAGTAATTAAGGTCTTTG GAGATGC | 145 | Adding MinD-tag to saat 3′ end |
| saat-MinD-R2 | CTAGAGGATCCCCGGGTACCGAGCTCTTAGGAACGT ACACCGAAAAATGATTTGATTTTAGCCATCATCCCTT TGTTTTG | 146 | |
| adh-A-F | TGAGGAGGGTTATTAAAATTATGGGTAGCGAAATTG CCGCGCTGG | 147 | CC-Di-A amplificati on |
| adh-A-R | ATTTTTCTCATCGATTTCATTCATATGGCTGCCGCGC GGCACCA | 148 | |
| B-nifJ-F1 | ACTGAAAAAGAAAAACGCTGCGCTGAAACAGAAAA TTGCCGCGCTGAAACAGATGAGAAAAATGAAAACTA TGGATG | 149 | CC-Di-B-nifJ amplificati on |
| B-nifJ-F2 | TTTAGGCCCTTAGGAGCTCGAAAGAGGGGTCAACGC GATGGGTAGCAAAATCGCCGCACTGAAAAAGAAAA ACGCTGCG | 150 | |
| B-nifJ-R | TCGACTCTAGAGGATCCCCGGGTACCCTATTGTTGAT TAGCTAACTTCTTG | 151 | |
| B-bdhA-F1 | ACTGAAAAAGAAAAACGCTGCGCTGAAACAGAAAA TTGCCGCGCTGAAACAGATGATGAGATTTACATTAC CAAGAG | 152 | CC-Di-B-bdhA amplificati on |
| B-bdhA-F2 | GTTAGCTAATCAACAATAGGAGGAGGGTTATTAAAA TTATGGGTAGCAAAATCGCCGCACTGAAAAAGAAAA ACGCTGCG | 153 | |
| B-bdhA-R | TCGACTCTAGAGGATCCCCGGGTACCTTAAAAATCA ACCTTATTTCCATAA | 154 | |
| pMTL-TJ1-F | TTGTAAAACGACGGCCAGTGGTAGACTTTAAGGATG GAACCTTTG | 155 | Amplificati on of fragments from pMTL8215 1-based plasmids |
| pMTL-TJ1-R | TTCTAACGCGTCACCTAAAGAGATCTCCATGGACGC GTGACGTCG | 156 | |
| rex-g1-all-F | AAGAAGAAAATAGAAATttgacagctagctcagtcctaggtataatG CTAGCTTGGAGATTTCGGACAACAAGTTTTAGAGCT AGAAATA | 157 | pYW34-Δrex |
| rex-g1-all-R | TATTTCTAGCTCTAAAACTTGTTGTCCGAAATCTCCA AGCTAGCATTATACCTAGGACTGAGCTAGCTGTCAA ATTTCTATTTTCTTCTT | 158 | |
| rex-Up-F | tatgactaataattagcggccgcGATATGTAGAGGATGTGATCAA GCTTT | 159 | |
| rex-Up-R | CCAAATAGCTCTTACTCCGCCTTCGCTTTAAATCCCT ATTGTCCATCT | 160 | |
| rex-Down-F | TGGACAATAGGGATTTAAAGCGAAGGCGGAGTAAG AGCTATTTGG | 161 | |
| rex-Down-R | gtaaccatcacactggcggccgcTCTGAAATATCTGCTCCAGCAA CAA | 162 | |
| ldh1-g1-all-F | AAGAAGAAAATAGAAATttgacagctagctcagtcctaggtataatG CTAGCTCAAGATTAAAATATGTCGTGTTTTAGAGCTA GAAATA | 163 | pYW34-Δldh1::P_{adh} -atf1-MinD |
| ldh1-g1-all-R | TATTTCTAGCTCTAAAACACGACATATTTTAATCTTG AGCTAGCATTATACCTAGGACTGAGCTAGCTGTCAA ATTTCTATTTTCTTCTT | 164 | |
| ZH-ldh1-Up-F | tatgactaataattagcggccgcTCTGCCAGTTGTCACTTGAGGTT TT | 165 | |
| ZH-ldh1-Up-R | GCTCTGTATTTGCTATCTCCTGCCATTACTTCTCCCAT AGCTTTATCCAT | 166 | |
| ZH-ldh1-Down-F | ATTTTTCGGTGTACGTTCCTAAGGAGTTGTAGGAATT GTAAATCCAGTG | 167 | |
| ZH-ldh1-Down-R | gtaaccatcacactggcggccgcCTTGTGTTTCTCTATCTGATGCT GGA | 168 | |
| atf1-ZH-ldh1-F | AAAGCTATGGGAGAAGTAATGGCAGGAGATAGCAA ATACAGAGCTA | 169 | |
| atf1-ZH-ldh1-R | GATTTACAATTCCTACAACTCCTTAGGAACGTACACC GAAAAATGATTTGATTTT | 170 | |
| lacZ1-g1-all-F | AAGAAGAAAATAGAAATttgacagctagctcagtcctaggtataatG CTAGCTATAGCCTTTGTGACCGTGAGTTTTAGAGCTA GAAATA | 171 | pYW34-ΔlacZ1::P_{ad} ₕ-atf1- MinD |
| lacZ1-g1-all-R | TATTTCTAGCTCTAAAACTCACGGTCACAAAGGCTAT AGCTAGCATTATACCTAGGACTGAGCTAGCTGTCAA ATTTCTATTTTCTTCTT | 172 | |
| lacZ1-Up-N-F | tatgactaataattagcggccgcCTTGTAGTCATTTCAGCGTGTTC CG | 173 | |
| lacZ1-Up-N-R | TCTTTAATTGCCACTTCTTGAAGCGGCCGCTCTCCTT TGAACATACCCTCTCCAC | 174 | |
| lacZ1-Down-N-F | GGAGAGGGTATGTTCAAAGGAGAGCGGCCGCTTCAA GAAGTGGCAATTAAAGAGGG | 175 | |
| lacZ1-Down-N-R | gtaaccatcacactggcggccgcCTCCCATGGTACCTCCTTATTG AAA | 176 | |
| atf1-ZH-lacZ1-F | tatgttcaaaggagagcggccgcCAGGAGATAGCAAATACAGAG | 177 | |
| | CTA | | |
| atf1-ZH-lacZ1-R | ttgccacttcttgaagcggccgcTTAGGAACGTACACCGAAAAAT GATTTGATTTT | 178 | |
| lacZ2-g1-all-F | AAGAAGAAAATAGAAATttgacagctagctcagtcctaggtataatG CTAGCAAGTATCCTAAATTACAAGGGTTTTAGAGCT AGAAATA | 179 | pYW34-ΔlacZ2::P_{ad} ₕ-atf1- MinD |
| lacZ2-g1-all-R | TATTTCTAGCTCTAAAACCCTTGTAATTTAGGATACT TGCTAGCATTATACCTAGGACTGAGCTAGCTGTCAA ATTTCTATTTTCTTCTT | 180 | |
| lacZ2-Up-N-F | ctttgtgatatgactaataattaACACGAGATATAGACGAAAGGAG G | 181 | |
| lacZ2-Up-N-R | TATTCCATGCTGCTTAAAATCTGCGGCCGCATTGCTG CCATCATTTCCTTTAGG | 182 | |
| lacZ2-Down-N-F | TAAAGGAAATGATGGCAGCAATGCGGCCGCAGATTT TAAGCAGCATGGAATAGGC | 183 | |
| lacZ2-Down-N-R | atccactagtaaccatcacactgGCAAAACCTGGTGTTAAACATC CTT | 184 | |
| atf1-ZH-lacZ2-F | aatgatggcagcaatgcggccgcCAGGAGATAGCAAATACAGAG CTA | 185 | |
| atf1-ZH-lacZ2-R | tgctgcttaaaatctgcggccgcTTAGGAACGTACACCGAAAAAT GATTTGATTTT | 186 | |
| atoD1ctfB1-g1-all-F | AAGAAGAAAATAGAAATttgacagctagctcagtcctaggtataatG CTAGCTCTTGGAACTGAAATTGAAAGTTTTAGAGCTA GAAATA | 187 | pYW34-ΔctfA1-ctfB1 |
| atoD1ctfB1-g1-all-R | TATTTCTAGCTCTAAAACTTTCAATTTCAGTTCCAAG AGCTAGCATTATACCTAGGACTGAGCTAGCTGTCAA ATTTCTATTTTCTTCTT | 188 | |
| atoD1ctfB1-Up-N-F | ctttgtgatatgactaataattaAGGGGTTAGTTACGCTTCGCCT | 189 | |
| atoD1ctfB1-Up-N-R | TCCACCACTTACTCTATCCTTCTGCGGCCGCATAATG GAGCCTCAATGGAAGCGATA | 190 | |
| atoD1ctfB1-Down-N-F | CGCTTCCATTGAGGCTCCATTATGCGGCCGCAGAAG GATAGAGTAAGTGGTGGATTT | 191 | |
| atoD1ctfB1-Down-N-R | atccactagtaaccatcacactgTATCCAAAGTTGTAAACCTCCTA ACG | 192 | |

TABLE S3

Butyl acetate production in the mutant strains with prophage deleted from the chromosome

| | FJ-1201 | FJ-1201 | FJ-1301 | FJ-1301 |
|---|---|---|---|---|
| | 48 h | 72 h | 48 h | 72 h |
| EA | <0.01 | <0.01 | <0.01 | <0.01 |
| BA | 19.3±0.5 | 19.7±1.7 | 19.1±0.5 | 19.4±0.7 |
| BB | 0.9±0.1 | 0.7±0.1 | 0.8±0.0 | 0.7±0.0 |
| BA (aqueous phase) | 0.6±0.1 | 0.6±0.1 | 0.5±0.0 | 0.5±0.1 |

*EA: ethyl acetate; BA: butyl acetate; BB: butyl butyrate. All values are in g/L.

TABLE S4

FJ-1201 ester production using biomass hydrolysates as the substrate with or without supplementation of exogenous organic nitrogen source*

| | 0Y+0T^{#} | 1Y+3T | 2Y+6T |
|---|---|---|---|
| EA | <0.01 | <0.01 | <0.01 |
| BA | 17.5±0.2 | 16.9±0.5 | 16.2±0.7 |
| BB | 0.1±0.0 | 0.2±0.1 | 0.2±0.1 |
| BA (aqueous phase) | 0.3±0.0 | 0.2±0.0 | 0.2±0.0 |

*EA: ethyl acetate; BA: butyl acetate; BB: butyl butyrate. All values are in g/L.
^{#}Y=Yeast, T=Typtone; 0Y+0T: 0 g/L Y and 0 g/L T; 1Y+3T: 1 g/L Y and 3 g/L T; 2Y+6T: 2 g/L Y and 6 g/L T.

TABLE S5

Project total capital investment ($ million) for the processes

| | Purchased Cost | Installed cost |
|---|---|---|
| Feedstock handling | 15.5 | 26.4 |
| Pretreatment & hydrolysis | 18.0 | 29.8 |
| Fermentation | 36.6 | 55.2 |
| Production recovery & upgrading | 6.6 | 12.6 |
| Product & chemical Storage | 1.3 | 2.2 |
| Wastewater treatment | 59.8 | 59.8 |
| Electricity & steam generation | 34.4 | 68.4 |
| Utilities | 4.5 | 8.3 |
| Total Installed Equipment cost | | 262.7 |
| Other Direct Cost | | 17.1 |
| Total Direct Cost (TDC) | | 279.8 |
| Total Indirect Costs (TIC) | | 167.9 |
| Fixed Capital Investment (FCI) | | 447.6 |
| Land | | 1.8 |
| Working Capital | | 22.4 |
| Total Capital Investment (TCI) | | 471.8 |

TABLE S6

Key operation parameters for the corn stover conversion and butyl acetate fermentation considered in the techno-economic analysis (TEA)

| | |
|---|---|
| Pretreatment and hydrolysis | |
| Sodium hydroxide loading | 40 kg/MT dry corn stover |
| Deacetylation incubation temperature | 80° C. |
| Disk milling energy consumption | 212 kWh/MT dry corn stover |
| Enzymatic hydrolysis solids loading | 20% |
| Enzyme loading | 19 mg protein/g cellulose |
| Cellulose hydrolysis efficiency | 82% |
| Hemicellulose hydrolysis efficiency | 74% |
| Hexadecane load | 1:1 by volume |
| Fermentation time | 96 hours |
| BA yield | 0.25 g BA/g consumed sugar |
| Butanol yield | 0.03 g butanol/g consumed sugar |
| Isopropanol yield | 0.04 g isopropanol/g consumed sugar |
| Additional nutrients | None (based on verified |
| | experiments) |

TABLE S7

Summary of key raw material costs

| Item | Cost ($) |
|---|---|
| Raw materials and utilities | |
| Corn stover (20% moisture) | 51.5/MT ^{a} |
| Sodium hydroxide | 116/MT ^{b} |
| Cellulase | 4,240/MT ^{a} |
| Hexadecane | 4,000/MT ^{b} |
| Wastewater treatment chemicals | 4,51/MT ^{b} |
| Boiler Chemicals | 8,336/MT ^{a} |
| Cooling tower chemicals | 3,668/MT ^{a} |
| Sulfuric acid | 41.0/MT ^{b} |
| Freshwater | 0.22/MT ^{a} |
| Electricity | 0.065/kWh ^{a} |
| Natural gas | 185/MT ^{b} |
| Co-product credits | |
| Butanol | 900/MT ^{b} |
| Isopropanol | 1150/MT ^{b} |
| Surplus Electricity | 0.065/kWh ^{b} |
| Fixed Operating Costs | |
| Labor costs | 2,500,000 ^{c} |
| Labor burden | 90% of labor cost |
| Maintenance | 3% of ISBL |
| Property insurance | 0.7% of fixed capital investment |

^{a}The price of corn stover and other chemicals were from the previous literature including Humbird et al. (2011)¹⁰, Chen et al. (2015)⁹, and Dalle Ave and Adams (2018)²⁹;
^{b}Data from different sources, including the ICIS chemical price report and industrial quotes;
°Assuming 50 employees with an average annual salary of $50,000 per employee.

TABLE S8

Butyl acetate production in the engineered strains for the evaluation of the effect of gene codon optimization

| | FJ-004 | FJ-007 |
|---|---|---|
| EA | <0.01 | <0.01 |
| BA | 5.5±0.5 | 5.0±0.2 |
| BB | 0.01±0.00 | < 0.01 |
| Glucose consumption | 79.3±0.1 | 76.7±2.4 |
| Lactate | 0.00 | 0.7±0.1 |
| Acetate | 0.3±0.00 | 0.2±0.1 |
| Ethanol | 1.0±0.0 | 1.0±0.0 |
| Acetone | 5.0±0.2 | 3.6±0.2 |
| Butyrate | 0.1±0.0 | 0.00 |
| Butanol | 7.8±0.0 | 9.5±0.7 |

* EA: ethyl acetate; BA: butyl acetate; BB: butyl butyrate. All values are in g/L.

### SUPPLEMENTAL REFERENCES

### References

The following references are provided as an aid in understanding the subject matter provided above. No admission is made that any of the following meet the legal definition of “prior art” in any country, or that any of the following are relevant to the patentability of anything that is claimed.

1. Pyne, M.E., Moo-Young, M., Chung, D.A. & Chou, C.P. Development of an electrotransformation protocol for genetic manipulation of Clostridium pasteurianum. Biotechnology for biofuels 6, 50 (2013).

2. Pyne, M.E., Bruder, M.R., Moo-Young, M., Chung, D.A. & Chou, C.P. Harnessing heterologous and endogenous CRISPR-Cas machineries for efficient markerless genome editing in Clostridium. Sci Rep 6, 25666 (2016).

3. Wang, Y. et al. Bacterial genome editing with CRISPR-Cas9: deletion, Integration, single nucleotide modification, and desirable “clean” mutant selection in Clostridium beijerinckii as an example. ACS synthetic biology 5, 721-732 (2016).

4. Zhang, J., Zong, W., Hong, W., Zhang, Z.-T. & Wang, Y. Exploiting endogenous CRISPR-Cas system for multiplex genome editing in Clostridium tyrobutyricum and engineer the strain for high-level butanol production. Metabolic engineering (2018).

5. Herman, N.A. et al. Development of a high-efficiency transformation method and implementation of rational metabolic engineering for the industrial butanol hyperproducer Clostridium saccharoperbutylacetonicum strain N1-4. Applied and environmental microbiology 83, e02942-02916 (2017).

6. Wang, S., Dong, S., Wang, P., Tao, Y. & Wang, Y. Genome editing in Clostridium saccharoperbutylacetonicum N1-4 with the CRISPR-Cas9 system. Applied and Environmental Microbiology 83, e00233-00217 (2017).

7. Ogata, S., Mihara, O., Ikeda, Y. & Hongo, M. Inducible phage tail-like particles of Clostridium saccharoperbutylacetonicum and its related strains. Agricultural and Biological Chemistry 36, 1413-1421 (1972).

8. Nale, J.Y. et al. Diverse temperate bacteriophage carriage in Clostridium difficile 027 strains. PloS one 7, e37263 (2012).

9. Chen, X. et al. Techno-economic analysis of the deacetylation and disk refining process: characterizing the effect of refining energy and enzyme usage on minimum sugar selling price and minimum ethanol selling price. Biotechnol Biofuels 8, 173 (2015).

10. D. Humbird, R.D., L. Tao, C. Kinchin,, D. Hsu, A.A., P. Schoen, J. Lukas, B. Olthof, M. Worley, & D. Sexton, a.D.D. Process design and economics for biochemical conversion of lignocellulosic biomass to ethanol:dilute-acid pretreatment and enzymatic hydrolysis of corn stover. (No. NREL/TP-5100-47764). National Renewable Energy Lab.(NREL), Golden, CO (United States). (2011).

11. Haas, M.J., McAloon, A.J., Yee, W.C. & Foglia, T.A. A process model to estimate biodiesel production costs. Bioresour Technol 97, 671-678 (2006).

12. Tao, L. & Aden, A. The economics of current and future biofuels. In Vitro Cellular & Developmental Biology- Plant 45, 199-217 (2009).

13. Feng, J. et al. Enhancing poly-γ-glutamic acid production in Bacillus amyloliquefaciens by introducing the glutamate synthesis features from Corynebacterium glutamicum. Microbial cell factories 16, 88 (2017).

14. Mittal, P., Brindle, J., Stephen, J., Plotkin, J.B. & Kudla, G. Codon usage influences fitness through RNA toxicity. Proceedings of the National Academy of Sciences 115, 8639-8644 (2018).

15. HONGO, M., MURATA, A. & OGATA, S. Bacteriophages of Clostridium saccharoperbutylacetonicum: Part XVI. Isolation and Some Characters of a Temperate Phage. Agricultural and Biological Chemistry 33, 337-342 (1969).

16. Zhou, Y., Liang, Y., Lynch, K.H., Dennis, J.J. & Wishart, D.S. PHAST: a fast phage search tool. Nucleic acids research 39, W347-W352 (2011).

17. Ogata, S., Nagao, N., Hidaka, Z. & Hongo, M. Bacteriophages of Clostridium saccharoperbutylacetonicum: Part XVII. The structure of phage HM 2. Agricultural and Biological Chemistry 33, 1541-1552 (1969).

18. Noh, H.J., Woo, J.E., Lee, S.Y. & Jang, Y.-S. Metabolic engineering of Clostridium acetobutylicum for the production of butyl butyrate. Applied microbiology and biotechnology, 1-9 (2018).

19. Noh, H.J., Lee, S.Y. & Jang, Y.-S. Microbial production of butyl butyrate, a flavor and fragrance compound. Applied microbiology and biotechnology 103, 2079-2086 (2019).

20. Zhang, F., Carothers, J.M. & Keasling, J.D. Design of a dynamic sensor-regulator system for production of chemicals and fuels derived from fatty acids. Nature biotechnology 30, 354 (2012).

21. Kosaka, T., Nakayama, S., Nakaya, K., Yoshino, S. & Furukawa, K. Characterization of the sol operon in butanol-hyperproducing Clostridium saccharoperbutylacetonicum strain N1-4 and its degeneration mechanism. Bioscience, biotechnology, and biochemistry 71, 58-68 (2007).

22. Wang, Y. et al. Development of a gene knockout system using mobile group II introns (Targetron) and genetic disruption of acid production pathways in Clostridium beijerinckii. Applied and environmental microbiology 79, 5853-5863 (2013).

23. Gu, Y. et al. Curing the endogenous megaplasmid in Clostridium saccharoperbutylacetonicum N1-4 (HMT) using CRISPR-Cas9 and preliminary investigation of the role of the plasmid for the strain metabolism. Fuel 236, 1559-1566 (2019).

24. Williams, D.R., Young, D.I. & Young, M. Conjugative plasmid transfer from Escherichia coli to Clostridium acetobutylicum. Microbiology 136, 819-826 (1990).

25. Heap, J.T., Pennington, O.J., Cartman, S.T. & Minton, N.P. A modular system for Clostridium shuttle plasmids. Journal of microbiological methods 78, 79-85 (2009).

26. Wang, P., Feng, J., Guo, L., Fasina, O. & Wang, Y. Engineering Clostridium saccharoperbutylacetonicum for high level Isopropanol-Butanol-Ethanol (IBE) production from acetic acid pretreated switchgrass using the CRISPR-Cas9 system. ACS Sustainable Chemistry & Engineering 7, 18153-18164 (2019).

27. Layton, D.S. & Trinh, C.T. Expanding the modular ester fermentative pathways for combinatorial biosynthesis of esters from volatile organic acids. Biotechnology and bioengineering 113, 1764-1776 (2016).

28. Layton, D.S. & Trinh, C.T. Engineering modular ester fermentative pathways in Escherichia coli. Metabolic engineering 26, 77-88 (2014).

29. Dalle Ave, G. & Adams, T.A. Techno-economic comparison of Acetone-Butanol-Ethanol fermentation using various extractants. Energy Conversion and Management 156, 288-300 (2018).

### EXEMPLARY EMBODIMENTS

The following are non-limiting examples of specific embodiments of the subject matter disclosed above. This disclosure specifically but non-exclusively supports claims to these embodiments:

Emb. 1. A modified microorganism capable of butyl acetate (BA) production, the microorganism capable of expressing an alcohol acyl transferase (AAT); and comprising a butanol synthesis pathway.

Emb. 2. Any one of the microorganisms above, wherein the AAT is selected from one or more of: Vaat, Saat, Atf1, Eht1, and a functional homolog of any of the foregoing.

Emb. 3. Any one of the microorganisms above, wherein the AAT is Vaat having at least 70% sequence identity with SEQ ID NO: 1.

Emb. 4. Any one of the microorganisms above, wherein the AAT is Saat having at least 70% sequence identity with SEQ ID NO: 2.

Emb. 5. Any one of the microorganisms above, wherein the AAT is Atf1 having at least 70% sequence identity with SEQ ID NO: 3.

Emb. 6. Any one of the microorganisms above, wherein the AAT is Eht1 having at least 70% sequence identity with SEQ ID NO: 4.

Emb. 7. Any one of the microorganisms above, comprising an acetyl CoA synthesis pathway.

Emb. 8. Any one of the microorganisms above, comprising multiple nucleic acid sequences each encoding Atf1 or a functional homolog of Atf1.

Emb. 9. Any one of the microorganisms above, comprising multiple genomic nucleic acid sequences each encoding Atf1 or a functional homolog of Atf1.

Emb. 10. A modified microorganism capable of butyl butyrate (BB) production, the microorganism capable of expressing an alcohol acyl transferase (AAT); and comprising a butanol synthesis pathway and a butyryl coenzyme A synthesis pathway.

Emb. 11. The microorganism of embodiment 0, wherein the AAT is Eht1 or a functional homolog of Eht1.

Emb. 12. The microorganism of any one of embodiments 0-0, wherein the AAT is Saat or a functional homolog of Saat.

Emb. 13. Any one of the microorganisms above, comprising the AAT enzyme.

Emb. 14. Any one of the microorganisms above, comprising a nucleic acid encoding the AAT.

Emb. 15. Any one of the microorganisms above, comprising a genomic nucleic acid encoding the AAT.

Emb. 16. Any one of the microorganisms above, comprising a plasmid encoding the AAT.

Emb. 17. Any one of the microorganisms above, wherein the microorganism is a prokaryote.

Emb. 18. Any one of the microorganisms above, wherein the microorganism is fermentative.

Emb. 19. Any one of the microorganisms above, wherein the microorganism is a bacterium of genus Clostridium.

Emb. 20. Any one of the microorganisms above, wherein the microorganism is selected from Clostridium saccharoperbutylacetonicum, Clostridium beijerinckii, Clostridium pasteurianum, and Clostridium tyrobutyricum.

Emb. 21. Any one of the microorganisms above, capable of expressing Lipase B or a functional homolog of Lipase B.

Emb. 22. Any one of the microorganisms above, capable of expressing a functional homolog of Lipase B having at least 70% sequence identity with SEQ ID NO: 5.

Emb. 23. Any one of the microorganisms above, capable of expressing Lipase B or a functional homolog of Lipase B, and comprising an acetic acid synthesis pathway.

Emb. 24. Any one of the microorganisms above, capable of expressing Lipase B or a functional homolog of Lipase B, and comprising a butyric acid synthesis pathway.

Emb. 25. Any one of the microorganisms above, comprising a nucleic acid encoding Lipase B or a functional homolog of Lipase B.

Emb. 26. Any one of the microorganisms above, comprising a nucleic acid encoding Lipase B or a functional homolog of Lipase B having at least 70% sequence identity with SEQ ID NO: 5.

Emb. 27. Any one of the microorganisms above, having reduced or eliminated NuoG activity.

Emb. 28. Any one of the microorganisms above, capable of expressing a heterologous Sadh or a functional homolog thereof.

Emb. 29. Any one of the microorganisms above, comprising a heterologous nucleic acid encoding a Sadh or a functional homolog of a Sadh.

Emb. 30. Any one of the microorganisms above, comprising a sadh-hydG gene cluster.

Emb. 31. Any one of the microorganisms above, comprising an exogenous AAT gene operatively linked to promoter P_{adh}.

Emb. 32. Any one of the microorganisms above, comprising an exogenous AAT gene operatively linked to a promoter P_{adh} native to the microorganism.

Emb. 33. Any one of the microorganisms above, comprising an exogenous AAT gene operatively linked to promoter P_{ald}.

Emb. 34. Any one of the microorganisms above, comprising an exogenous AAT gene operatively linked to promoter P_{ald} native to the microorganism.

Emb. 35. Any one of the microorganisms above, comprising an AAT that is localized at the cell membrane.

Emb. 36. Any one of the microorganisms above, wherein the AAT is fused to a C-terminal membrane-targeting sequence of MinD.

Emb. 37. Any one of the microorganisms above, wherein the AAT is fused to a C-terminal membrane-targeting sequence of MinD encoded by SEQ ID NO: 6.

Emb. 38. Any one of the microorganisms above, wherein the AAT is fused to an 8-12 residue C-terminal membrane-targeting sequence of MinD.

Emb. 39. Any one of the microorganisms above, wherein the AAT is fused at its C-terminal end to the C-terminal membrane-targeting sequence of MinD.

Emb. 40. Any one of the microorganisms above, comprising a nucleic acid encoding a polypeptide comprising an alcohol acyltransferase and a C-terminal membrane-targeting sequence of MinD.

Emb. 41. Any one of the microorganisms above, wherein the microorganism has been cured of a prophage.

Emb. 42. Any one of the microorganisms above, wherein the microorganism has been cured of all native prophages.

Emb. 43. Any one of the microorganisms above, wherein the microorganism has been cured of one or more prophages by inactivation of an integrase gene of the one or more prophages.

Emb. 44. Any one of the microorganisms above, wherein the microorganism has been cured of one or more prophages by inactivation of an integrase gene of the one or more prophages through the partial or entire deletion of the integrase gene.

Emb. 45. Any one of the microorganisms above, wherein the microorganism has been cured of one or more prophages by deletion of the one or more prophages.

Emb. 46. Any one of the microorganisms above, wherein the microorganism is a bacterium of genus Clostridium, and wherein the microorganism has been cured of one or more of prophages P1, P2, P3, P4, and P5.

Emb. 47. Any one of the microorganisms above, wherein the microorganism is a bacterium of genus Clostridium, and wherein the microorganism has been cured of all of prophages P1, P2, P3, and P4.

Emb. 48. Any one of the microorganisms above, wherein said microorganism does not express a functional redox-sensing transcriptional repressor Rex.

Emb. 49. Any one of the microorganisms above, capable of expressing soluble pyridine nucleotide transhydrogenase (SthA) or a functional homolog thereof.

Emb. 50. Any one of the microorganisms above, capable of expressing soluble pyridine nucleotide transhydrogenase (SthA) having at least 70% sequence identity with SEQ ID NO: 7.

Emb. 51. Any one of the microorganisms above, wherein said microorganism does not express a functional cftA1-ctfB1 gene cluster.

Emb. 52. Any one of the microorganisms above, wherein the functional homolog has only exemplary substitutions from Table 1 compared to the AAT, Lipase B, SthA, or Sadh.

Emb. 53. Any one of the microorganisms above, wherein the functional homolog has only preferred substitutions from Table 1 compared to the AAT, Lipase B, SthA, or Sadh.

Emb. 54. Any one of the microorganisms above, wherein the functional homolog has a amino acid sequence identity level to the AAT, Lipase B, SthA, or Sadh of at least 70, 75, 70, 85, 90, 91, 92, 93, 94, 95, 96, 97, 98, 99, 99.5, 99.9, or 100%.

Emb. 55. A genetically modified Clostridium saccharoperbutylacetonicum having improved BA production and designated YM028P, having NCMA designation number 202012116, deposited on 16 Dec. 2020 at the National Center for Marine Algae and Microbiota at Bigelow Laboratory for Ocean Sciences, 60 Bigelow Drive, East Boothbay, ME 04544 USA.

Emb. 56. A genetically modified Clostridium saccharoperbutylacetonicum having improved BB production and designated YM016PB, having NCMA designation number 202012115, deposited on 16 Dec. 2020 at the National Center for Marine Algae and Microbiota at Bigelow Laboratory for Ocean Sciences, 60 Bigelow Drive, East Boothbay, ME 04544 USA.

Emb. 57. A genetically modified Clostridium saccharoperbutylacetonicum having improved BA production and designated FJ-1301, having NCMA designation number 202012114, deposited on 16 Dec. 2020 at the National Center for Marine Algae and Microbiota at Bigelow Laboratory for Ocean Sciences, 60 Bigelow Drive, East Boothbay, ME 04544 USA.

Emb. 58. A method of ester production, comprising culturing any one of the microorganisms above under conditions suitable to produce an ester.

Emb. 59. Any one of the methods above, comprising culturing any one of the microorganisms above in a medium containing glucose.

Emb. 60. Any one of the methods above, comprising culturing any one of the microorganisms above in a medium containing a biomass hydrolysate.

Emb. 61. Any one of the methods above, comprising culturing any one of the microorganisms above in a medium containing a corn stover hydrolysate.

Emb. 62. Any one of the methods above, wherein culturing occurs at mesophilic temperatures.

Emb. 63. Any one of the methods above, wherein culturing occurs under anaerobic conditions.

Emb. 64. Any one of the methods above, wherein the ester is at least one of BA and BB.

Emb. 65. Any one of the methods above, producing at least about 1.5 g BA/L of culture.

Emb. 66. Any one of the methods above, producing at least 1.5 g BA/L of culture.

Emb. 67. Any one of the methods above, producing at least about 5, 7.5, 10, 12.5, 13, 14, 15, 16, 17, 18, 19, 20, or 25 g BA/L of culture.

Emb. 68. Any one of the methods above, producing at least 5, 7.5, 10, 12.5, 13, 14, 15, 16, 17, 18, 19, 20, or 25 g BA/L of culture.

Emb. 69. Any one of the methods above, producing at least about 0.1 g BB/L of culture.

Emb. 70. Any one of the methods above, producing at least 0.1 g BB/L of culture.

Emb. 71. Any one of the methods above, producing at least about 0.2, 0.3, 0.4, 0.5, 0.6, 0.7, 0.8, 0.9, 1.0, 1.1, 1.2, 1.3, 1.4, 1.5. or 1.6 g BB/L of culture.

Emb. 72. Any one of the methods above, producing at least 0.2, 0.3, 0.4, 0.5, 0.6, 0.7, 0.8, 0.9, 1.0, 1.1, 1.2, 1.3, 1.4, 1.5. or 1.6 g BB/L of culture.

### CONCLUSIONS

It is to be understood that any given elements of the disclosed embodiments of the invention may be embodied in a single structure, a single step, a single substance, or the like. Similarly, a given element of the disclosed embodiment may be embodied in multiple structures, steps, substances, or the like. The foregoing description and accompanying drawings illustrate and describe certain processes, machines, manufactures, and compositions of matter, some of which embody the invention(s). Such descriptions or illustrations are not intended to limit the scope of what can be claimed, and are provided as aids in understanding the claims, enabling the making and use of what is claimed, and teaching the best mode of use of the invention(s). If this description and accompanying drawings are interpreted to disclose only a certain embodiment or embodiments, it shall not be construed to limit what can be claimed to that embodiment or embodiments. Any examples or embodiments of the invention described herein are not intended to indicate that what is claimed must be coextensive with such examples or embodiments. Where it is stated that the invention(s) or embodiments thereof achieve one or more objectives, it is not intended to limit what can be claimed to versions capable of achieving all such objectives. Any statements in this description criticizing the prior art are not intended to limit what is claimed to exclude any aspects of the prior art. Additionally, the disclosure shows and describes certain embodiments of the processes, machines, manufactures, compositions of matter, and other teachings disclosed, but it is to be understood that the teachings of the present disclosure are capable of use in various other combinations, modifications, and environments and is capable of changes or modifications within the scope of the teachings as expressed herein. Any section headings herein are provided only for consistency with the suggestions of 37 C.F.R. § 1.77 or otherwise to provideorganizational queues. These headings shall not limit or characterize the invention(s) set forth herein.

The following is claimed:

1. A modified microorganism capable of butyl acetate (BA) production, the microorganism capable of expressing an alcohol acyl transferase (AAT); and comprising a butanol synthesis pathway.

2. The modified microorganism of claim 1, wherein the AAT is selected from one or more of: Vaat, Saat, Atf1, Eht1, and a functional homolog of any of the foregoing.

3. The modified microorganism of claim 1, capable of expressing all of: Vaat, Saat, Atf1, and Eht1.

4. The modified microorganism of claim 1, wherein the AAT is Vaat having at least 70% sequence identity with SEQ ID NO: 1.

5. The modified microorganism of claim 1, wherein the AAT is Saat having at least 70% sequence identity with SEQ ID NO: 2.

6. The modified microorganism of claim 1, wherein the AAT is Atf1 having at least 70% sequence identity with SEQ ID NO: 3.

7. The modified microorganism of claim 1, wherein the AAT is Eht1 having at least 70% sequence identity with SEQ ID NO: 4.

8. The modified microorganism of claim 1, comprising an acetyl CoA synthesis pathway.

9. The modified microorganism of claim 1, comprising multiple nucleic acid sequences each encoding Atf1 or a functional homolog of Atf1.

10. A modified microorganism capable of butyl butyrate (BB) production, the microorganism capable of expressing an alcohol acyl transferase (AAT); and comprising a butanol synthesis pathway and a butyryl coenzyme A synthesis pathway.

11. The microorganism of claim 10, wherein the AAT is Eht1 or a functional homolog of Eht1.

12. The microorganism of claim 10, wherein the AAT is Saat or a functional homolog of Saat.

13. Any one of the microorganisms of claims 1 and 10, wherein the microorganism is a prokaryote.

14. Any one of the microorganisms of claims 1 and 10, wherein the microorganism is fermentative.

15. Any one of the microorganisms of claims 1 and 10, wherein the microorganism is a bacterium of genus Clostridium.

16. Any one of the microorganisms of claims 1 and 10, wherein the microorganism is selected from Clostridium saccharoperbutylacetonicum, Clostridium beijerinckii, Clostridium pasteurianum, and Clostridium tyrobutyricum.

17. Any one of the microorganisms of claims 1 and 10, capable of expressing Lipase B or a functional homolog of Lipase B.

18. Any one of the microorganisms of claims 1 and 10, capable of expressing a functional homolog of Lipase B having at least 70% sequence identity with SEQ ID NO: 5.

19. Any one of the microorganisms of claims 1 and 10, capable of expressing Lipase B or a functional homolog of Lipase B, and comprising an acetic acid synthesis pathway.

20. Any one of the microorganisms of claims 1 and 10, capable of expressing Lipase B or a functional homolog of Lipase B, and comprising a butyric acid synthesis pathway.

21. Any one of the microorganisms of claims 1 and 10, having reduced or eliminated NuoG activity.

22. Any one of the microorganisms of claims 1 and 10, capable of expressing a heterologous Sadh or a functional homolog thereof.

23. Any one of the microorganisms of claims 1 and 10, comprising a sadh-hydG gene cluster.

24. Any one of the microorganisms of claims 1 and 10, comprising an exogenous AAT gene operatively linked to promoter P_{adh}.

25. Any one of the microorganisms of claims 1 and 10, comprising an exogenous AAT gene operatively linked to promoter P_{ald}.

26. Any one of the microorganisms of claims 1 and 10, comprising an AAT that is localized at the cell membrane.

27. Any one of the microorganisms of claims 1 and 10, wherein the AAT is fused to a C-terminal membrane-targeting sequence of MinD.

28. Any one of the microorganisms of claims 1 and 10, wherein the AAT is fused to a C-terminal membrane-targeting sequence of MinD encoded by SEQ ID NO: 6.

29. Any one of the microorganisms of claims 1 and 10, wherein the AAT is fused to an 8-12 residue C-terminal membrane-targeting sequence of MinD.

30. Any one of the microorganisms of claims 1 and 10, wherein the microorganism has been cured of a prophage.

31. Any one of the microorganisms of claims 1 and 10, wherein the microorganism has been cured of all native prophages.

32. Any one of the microorganisms of claims 1 and 10, wherein the microorganism has been cured of one or more prophages by deletion of the one or more prophages.

33. Any one of the microorganisms of claims 1 and 10, wherein the microorganism is a bacterium of genus Clostridium, and wherein the microorganism has been cured of one or more of prophages P1, P2, P3, P4, and P5.

34. Any one of the microorganisms of claims 1 and 10, wherein the microorganism is a bacterium of genus Clostridium, and wherein the microorganism has been cured of all of prophages P1, P2, P3, P4, and P5.

35. Any one of the microorganisms of claims 1 and 10, wherein said microorganism does not express a functional redox-sensing transcriptional repressor Rex.

36. Any one of the microorganisms of claims 1 and 10, capable of expressing soluble pyridine nucleotide transhydrogenase (SthA) or a functional homolog thereof.

37. Any one of the microorganisms of claims 1 and 10, capable of expressing soluble pyridine nucleotide transhydrogenase (SthA) having at least 70% sequence identity with SEQ ID NO: 7.

38. Any one of the microorganisms of claims 1 and 10, wherein said microorganism does not express a functional cftA1-ctfB1 gene cluster.

39. Any one of the microorganisms of claims 1 and 10, wherein the functional homolog has only preferred substitutions from Table 1 compared to the AAT, Lipase B, SthA, or Sadh.

40. A genetically modified Clostridium saccharoperbutylacetonicum having improved BA production and designated YM028P, having NCMA designation number 202012116, deposited on 16 December 2020 at the National Center for Marine Algae and Microbiota at Bigelow Laboratory for Ocean Sciences, 60 Bigelow Drive, East Boothbay, ME 04544 USA.

41. A genetically modified Clostridium saccharoperbutylacetonicum having improved BB production and designated YM016PB, having NCMA designation number 202012115, deposited on 16 December 2020 at the National Center for Marine Algae and Microbiota at Bigelow Laboratory for Ocean Sciences, 60 Bigelow Drive, East Boothbay, ME 04544 USA.

42. A genetically modified Clostridium saccharoperbutylacetonicum having improved BA production and designated FJ-1301, having NCMA designation number 202012114, deposited on 16 December 2020 at the National Center for Marine Algae and Microbiota at Bigelow Laboratory for Ocean Sciences, 60 Bigelow Drive, East Boothbay, ME 04544 USA.

43. A method of ester production, comprising culturing any one of the microorganisms of claims 1, 10, and 40-42 under conditions suitable to produce an ester.

44. The method of claim 43, comprising culturing any one of the microorganisms above in a medium containing glucose.

45. The method of claim 43, comprising culturing any one of the microorganisms above in a medium containing a biomass hydrolysate.

46. The method of claim 43, comprising culturing any one of the microorganisms above in a medium containing a corn stover hydrolysate.

47. The method of claim 43, wherein the ester is at least one of BA and BB.

48. The method of claim 43, producing at least 1.5 g BA/L of culture.

49. The method of claim 43, producing at least 25 g BA/L of culture.

50. The method of claim 43, producing at least 0.1 g BB/L of culture.

51. The method of claim 43, producing at least 1.6 g BB/L of culture.
